(19) United States
(12) Patent Application Publication
(10) Pub. No.: US 2023/0146239 A1
(43) Pub. Date: May 11, 2023

(54) GUANIDINE-MODIFIED C-TERMINUS VANCOMYCIN COMPOUNDS, COMPOSITIONS AND METHODS

(71) Applicant: THE SCRIPPS RESEARCH INSTITUTE, La Jolla, CA (US)

(72) Inventors: Dale L. BOGER, La Jolla, CA (US); Zhi-Chen WU, San Diego, CA (US)

(73) Assignee: THE SCRIPPS RESEARCH INSTITUTE, La Jolla, CA (US)

(21) Appl. No.: 17/918,448

(22) PCT Filed: Apr. 13, 2021
(86) PCT No.: PCT/US2021/027133

§ 371 Date: Oct. 12, 2022

Related U.S. Application Data
(60) Provisional application No. 63/012,726, filed on Apr. 20, 2020.
(60) Provisional application No. 63/040,339, filed on Jun. 17, 2020.

(51) Int. Cl. C07K 9/00; A61P 31/04

(52) U.S. Cl. CPC C07K 9/008; A61P 31/04; A61K 38/00

(57) ABSTRACT

A series of vancomycin C-terminus guanidine modifications are disclosed that improve antimicrobial activity, enhance the durability of antimicrobial action against selection or induction of resistance, and provide two synergistic mechanisms of action independent of D-Ala-D-Ala binding that cause inhibition of cell wall biosynthesis, while inducing bacterial cell permeability. A contemplated compound contains two combined peripheral modifications, a (4-chlorobiphenyl)methyl (CBP) and C-terminus guanidine modification, that provide new treatments against not only vancomycin-sensitive, but especially vancomycin-resistant bacteria. The data demonstrate that the synergistic behavior of the peripheral modifications requires the presence of both the CBP and guanidine modifications in a single molecule versus their combined use as an equimolar mixture of singly modified compounds. A prototypical member of the series, G3-CBP-vancomycin (15), exhibits no hemolytic activity, displays no mammalian cell growth inhibition, and possesses improved and especially attractive in vivo pharmacokinetic (PK) properties.

### STATEMENT OF GOVERNMENT LICENSE RIGHTS

This invention was made with government support under grant number CA041101 awarded by the National Institutes of Health. The government has certain rights in the invention.

### TECHNICAL FIELD

The present invention is directed to vancomycin derivatives modified at the molecule's C-terminus with a guanidine group, compositions containing such a compound and methods of using a compound for inhibiting bacterial cell growth, particularly in bacteria that are not sensitive to vancomycin.

### BACKGROUND ART

Vancomycin (Compound 1) and related glycopeptide antibiotics are one of the most important class of natural product drugs¹⁻⁵. As the first member of the class, vancomycin has been used in the clinic for over 60 years and most recently as the antibiotic of the last resort for the treatment of infections caused by resistant Gram-positive pathogens, including methicillin-resistant S. aureus (MRSA)⁶⁻⁷. Vancomycin binds to the C-terminus D-Ala-D-Ala moiety of bacterial cell wall precursors and inhibits cell wall biosynthesis⁸⁻⁹.

Clinical resistance to vancomycin was initially observed after 30 years of extensive use¹⁰⁻¹², first in enterococci (VRE) followed by the recent emergence of vancomycin-resistant S. aureus (VRSA)¹³⁻¹⁴, which rank 4^{th} and 5^{th} on the WHO global priority list of antibiotic-resistant bacteria treats¹⁵. The mechanism of vancomycin clinical resistance found in these pathogens is a late stage remodeling of the bacteria cell wall precursor C-termini from D-Ala-D-Ala to D-Ala-D-Lac, reducing vancomycin binding affinity (1000-fold) and antimicrobial potency (1000-fold) 16-18.

This widespread (VRE) and emerging (VRSA) resistance presents an urgent need for the development of next generation glycopeptide antibiotics to overcome such resistance. In order to improve potency of the antibiotics against sensitive/resistant strains, peripheral modifications on vancomycin and related glycopeptides have been extensively investigated and some have been shown to enhance antibacterial potency or efficacy¹⁹⁻²⁰, introduce additional mechanisms of action²¹⁻²⁴, or improve pharmacokinetic properties²⁵. As a result, three second generation glycopeptide antibiotics, telavancin²⁶, dalbavancin²⁷, and oritavancin²⁸, have been recently approved for clinical use. The structural formulas for those compounds as are shown below, along with vancomycin (Compound 1).

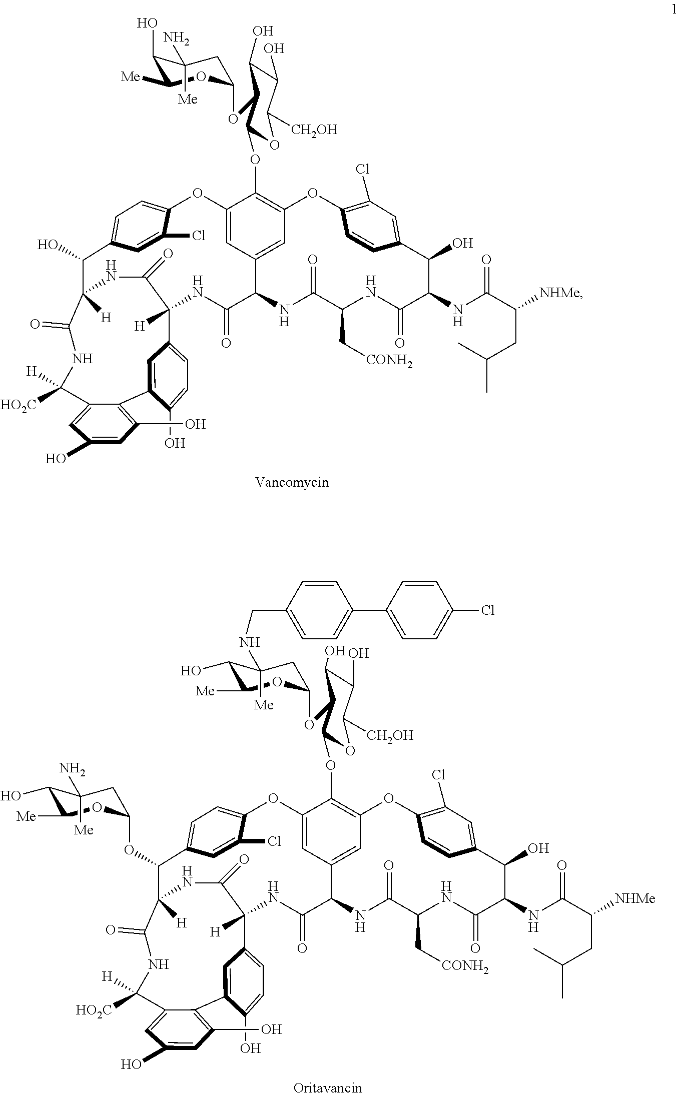

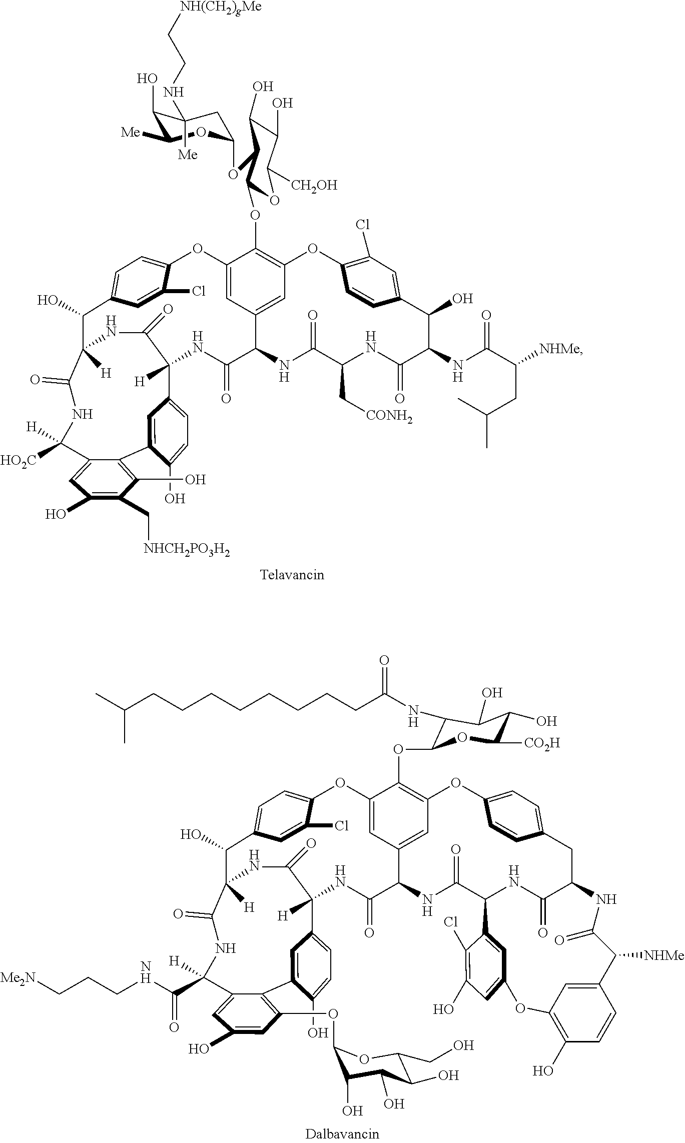

A new peripheral modification on vancomycin was recently reported having a C-terminus quaternary trimethylammonium cation, which was found to enhance the antimicrobial potency of vancomycin and its analogues by introducing an additional mechanism of action (bacteria cell envelope permeabilization)²⁹. This trimethylammonium cation modification was combined with a key vancomycin binding pocket modification that conveys dual D-Ala-D-Ala/D-Ala-D-Lac binding and directly overcomes the intrinsic molecular basis of vancomycin resistance³⁰⁻³³, and a well-known peripheral (4-chlorobiphenyl)methyl (CBP) modification that was established to inhibit transglycosylase and cell wall biosynthesis independent of D-Ala-D-Ala/D-Ala-D-Lac bindine³⁴⁻³⁵. This work provided compounds we named for convenience as C1-vancomycin (Compound 3) and as C1-CBP-vancomycin (Compound 4) whose structural formulas are shown below.

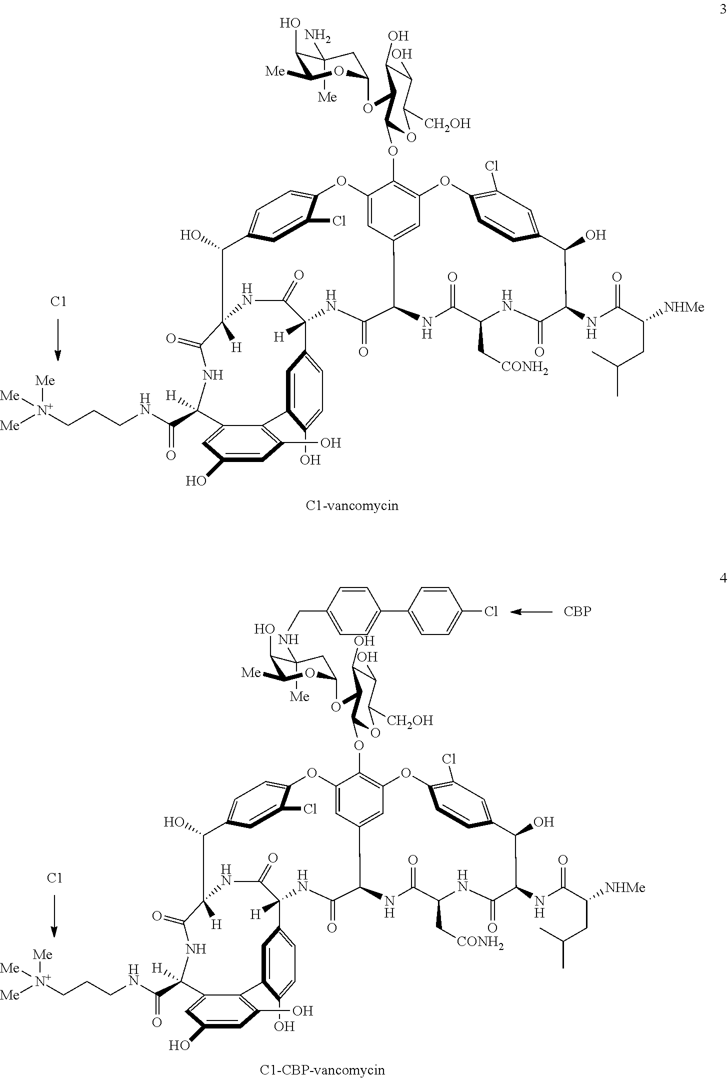

These modifications provided antibiotics bearing multiple independent synergistic mechanisms of action that are highly potent against both vancomycin-sensitive and vancomycin-resistant pathogens with an even more enhanced durability. Further investigation into this added quaternary trimethylammonium cation resulted in the observation of the structure and site-specific^{29,36-37} nature of the modification and its conveyance of in vivo efficacy against VRSA and improved pharmacokinetic (PK) properties³⁸. This report included a documentation of the improved antibiotic durability, identification of a candidate cell membrane target responsible for the added functional activity, and demonstration that it conveyed improved physical (e.g., solubility) and pharmacological (e.g., PK) properties without introducing acute liabilities.

The observed improvement in the properties of vancomycin and its derivatives by the presence of a simple quaternary trimethylammonium cation modification, which appears to serve as a permanently charged surrogate for a reversibly protonated dimethylamine, inspired further exploration of related peripheral modifications. This exploration was addressed by targeting modifications to vancomycin and (4-chlorobiphenyl)methyl-vancomycin (Compound 2, CBP-vancomycin) to provide analogues capable of expressing activity through as many as three independent and synergistic mechanisms of action, two of which are not dependent on D-Ala-D-Ala/D-Ala-D-Lac binding.

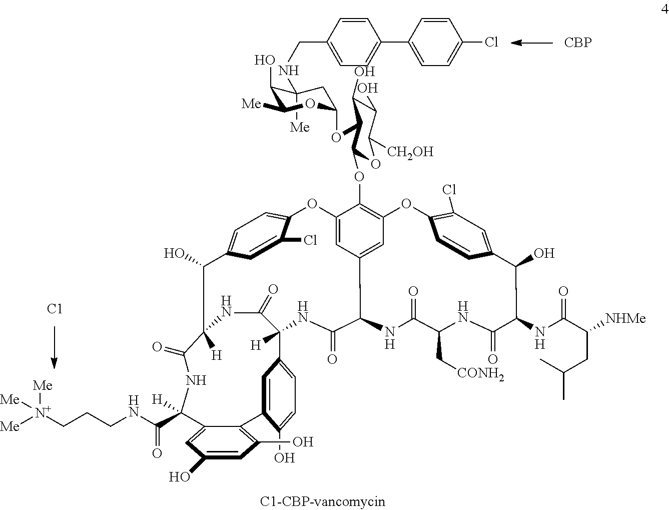

A protonated guanidinium group (pKₐ=13.2) could act as a better, more persistent positive charge under physiological conditions compared with tertiary amine (pKₐ=10.6). In contrast to a quaternary trimethylammonium cation, a guanidinium group can also serve as a multiple hydrogen-bond donor, increasing binding affinity to anionic groups of biomolecules.

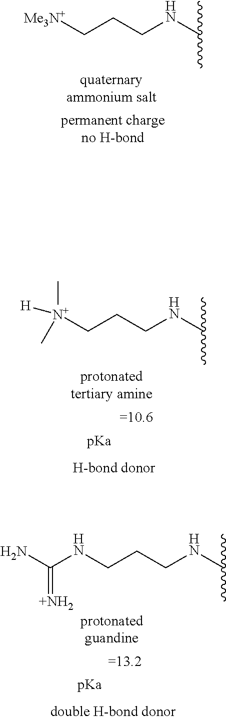

In particular, phosphates found in the phospholipid/teichoic acid³⁹ components of the cell envelope of Gram-positive pathogens implicated in studies with Compound 4 may serve as potential binding sites for positively charged groups where a guanidinium cation would be expected to display improved interactions. The disclosure that follows highlights the unexpected benefits of the presence of a C-terminal guanidinium group on the properties of a CBP-vancomycin as compared to the presence of a quaternary ammonium group.

### SUMMARY

One aspect of the present invention contemplates a compound that corresponds in structure to structural Formula I, below, in which X=H,H, S, O or NH; and Circle A,

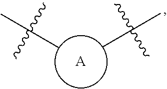

is a linking moiety that has a length of equal to greater than a saturated chain

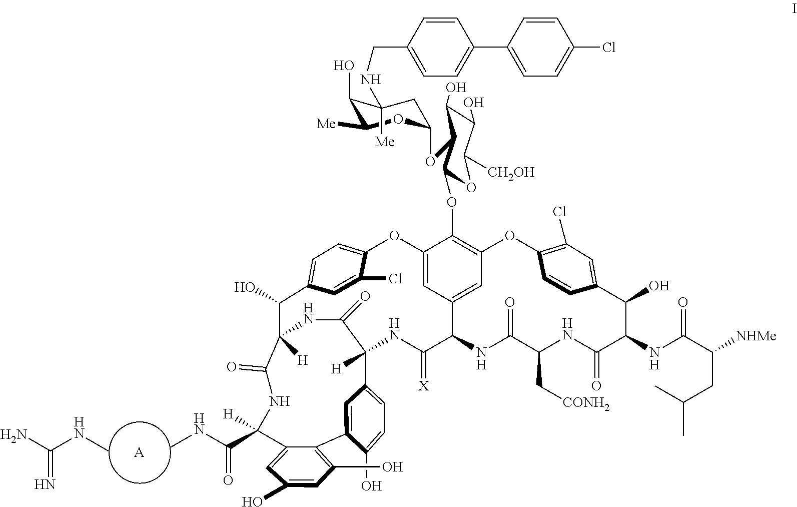

of 2 carbon atoms and less than a saturated chain of about 12 carbon atoms. A pharmaceutically acceptable salt of such a compound is also contemplated.

The constituents of a Circle A linker moiety can be quite varied. The atom chain that connects the guanidino and amido nitrogen atoms as shown in Formula I, above, can be hydrocarbyl, can contain one to three oxyethylene or N-methylaminoethylene groups, an aromatic or aliphatic carbocyclic or heterocyclic ring.

A Circle A linker moiety can also be comprised of two linker rings Circle B and Circle C that can be bonded directly together by one bond or by two bonds, thereby forming a fused ring, or indirectly bonded together. The Circle B and Circle C rings can also be linked indirectly via a sub-linker Z group of one to a chain of four atoms. The Circle B and Circle C rings can also be the same or different, and can be aromatic or aliphatic carbocyclic or heterocyclic rings.

A contemplated compound of Formula I or its pharmaceutically acceptable salt is typically found present dissolved or dispersed in a pharmaceutical composition. The compound is present in an effective antibacterial amount.

A compound of this invention is particularly effective when used in a method of treating a bacterially-infected mammal in need of antibacterial treatment. That method contemplates administering an antibacterial-effective amount of a compound of Formula I or a pharmaceutically acceptable salt of such a compound to said infected mammal in need.

### BRIEF DESCRIPTION OF THE DRAWINGS

In the drawings forming a part of this disclosure,

FIGS. 1A and 1B are graphs that illustrate acquisition resistance of bacteria on serial passaging of two strains of VanA VRE in the presence of 0.5×MIC levels of compound: CBP-vancomycin (Compound 2), G3-vancomycin (Compound 5) and G3-CBP-vancomycin (Compound 15). Examining the X-axis for each graph above the 50 passages mark, the line nearest to the X-axis represents data for Compound 15, the middle line represents data for Compound 5, and the topmost line represents data for Compound 2. FIGS. 1C and 1D are graphs that include the data from FIGS. 1A and 1B as well as data for the resistance acquisition on serial passaging of two strains of VanA VRE in the presence of 0.5×MIC levels of frontline therapy compounds daptomycin, linezolid and tigecycline. Examining the X-axis for each graph above the 40 passages mark, the line nearest to the X-axis represents data for Linezolid, the next line up represents data for Tigecycline, and the topmost line represents data for Daptomycin.

FIGS. 2A and 2B are graphs that illustrate cell membrane permeability induced by Compounds 1, 2, 4, 5, 15 and 19 (10 μM added at 5 minutes) and no antibiotic. In each figure, the compound number (4, 15 and 19) whose data produced the three upper lines is shown adjacent to each appropriate line, whereas the data for the other three compounds (1, 2, and 5) and no antibiotic were too similar to distinguish.

FIGS. 3A and 3B are graphs that illustrate cell membrane permeability induced by Compounds 1, 2, 15 (10 μM added at 5 minutes) and compound 15 in the presence of 100 μg/mL exogeneous LTA in VanA VRE E. faecalis BM 4166 and E. faecium ATCC BAA-2317, respectively. In each figure, the compound number (1, 2, 15 and 15+LTA) whose data produced each line is shown adjacent to its line.

FIG. 4 is a graph of the results of an assay of red blood cell hemolysis percent observed versus concentration expressed as fold concentration over measured MIC of G3-vancomycin (Compound 5) and G3-CBP-vancomycin (Compound 15) alongside vancomycin (Compound 1) and C1-CBP-vancomycin (Compound 4).

FIGS. 5A and 5B are graphs that illustrate the efficacy of G3-CBP-vancomycin (Compound 15) and linezolid against the multidrug resistant and vancomycin-resistant S. aureus (VRSA) strain VRS-2 in the mouse thigh infection model (n=5/dose). Compound 15 and control vehicle were administered sc once at 2 hours at the doses indicated. The reference standard linezolid was administered orally (po) twice at 2 and 12 hours at 50 mg/kg. FIG. 5A: dose-dependent reduction in bacterial load. FIG. 5B: dose-dependent bactericidal effect (relative to 2 hours baseline).

The present invention provides several benefits and advantages compared to vancomycin itself and vancomycin analogues know in the art.

Thus, one benefit of a contemplated guanidine-containing glycoside-modified vancomycin analogue is activity against both Gram positive and Gram-negative bacteria.

An advantage of a contemplated guanidine-containing glycoside-modified vancomycin analogue is enhanced activity against vancomycin-resistant bacteria as compared to other vancomycin analogues.

Another benefit of a contemplated compound is superior durability against the development of bacterial resistance to a compound's anti-bacterial activity over 50 bacterial growth passages that surpasses that of vancomycin, vancomycin analogues containing only a single modification at the glycoside of C-terminus of the molecule, and also an analogue containing a glycoside modification and a C-terminal amido-linked quaternary ammonium group.

Another advantage of a contemplated guanidine-containing glycoside-modified vancomycin analogue is that such compounds appear to be benign toward mammal cells, including blood cells that showed no hemolysis at more than 100-times the MIC.

A still further benefit of a contemplated compound is its superior durability against the development of bacterial resistance to a compound's anti-bacterial activity over 50 bacterial growth passages that surpasses commercial front line products daptomycin, linezolid and tigecycline.

### Definitions

In the context of the present invention and the associated claims, the following terms have the following meanings:

The articles “a” and “an” are used herein to refer to one or to more than one (i.e., to at least one) of the grammatical object of the article. By way of example, “an element” means one element or more than one element.

Each of the patents, patent applications and articles cited herein is incorporated by reference.

The word “hydrocarbyl” is used herein as a short hand term for a non-aromatic group that includes straight and branched chain aliphatic as well as alicyclic groups or radicals that contain only carbon and hydrogen. Thus, alkyl, alkenyl and alkynyl groups are contemplated, whereas aromatic hydrocarbons such as phenyl.

Where a specific aliphatic hydrocarbyl substituent group is intended, that group is recited; i.e., C₁-C₄ alkyl, methyl or tert-butyl. Exemplary hydrocarbyl groups contain a chain of 1 to 4 carbon atoms, and preferably 1 or 2 carbon atoms.

A particularly preferred hydrocarbyl group is an alkyl group. As a consequence, a generalized, but more preferred substituent can be recited by replacing the descriptor “hydrocarbyl” with “alkyl” in any of the substituent groups enumerated herein.

Examples of alkyl radicals include methyl, ethyl, n-propyl, isopropyl, n-butyl, isobutyl, sec-butyl, tert-butyl. Examples of suitable alkenyl radicals include ethenyl (vinyl), 2-propenyl, 3-propenyl, 1,4-butadienyl, 1-butenyl, 2-butenyl, and 3-butenyl. Examples of alkynyl radicals include ethynyl, 2-propynyl, 1-propynyl, 1-butynyl, 2-butynyl, 3-butynyl, and 1-methyl-2-propynyl.

As a skilled worker will understand, a substituent that cannot exist such as a C₁ alkenyl group is not intended to be encompassed by the word “hydrocarbyl”, although such substituents with two or more carbon atoms are intended.

Usual chemical suffix nomenclature is followed when using the word “hydrocarbyl” except that the usual practice of removing the terminal “yl” and adding an appropriate suffix is not always followed because of the possible similarity of a resulting name to one or more substituents. Thus, a hydrocarbyl ether is referred to as a “hydrocarbyloxy” group rather than a “hydrocarboxy” group as may possibly be more proper when following the usual rules of chemical nomenclature. Illustrative hydrocarbyloxy groups include methoxy, ethoxy, n-propoxy, isopropoxy, allyloxy, n-butoxy, iso-butoxy, sec-butoxy, and tert-butoxy groups.

### DETAILED DESCRIPTION OF PREFERRED EMBODIMENTS

A compound contemplated by this invention corresponds in structure to structural Formula I, below, in which X=H,H, S, O or NH; and Circle A,

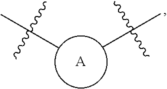

is a linking

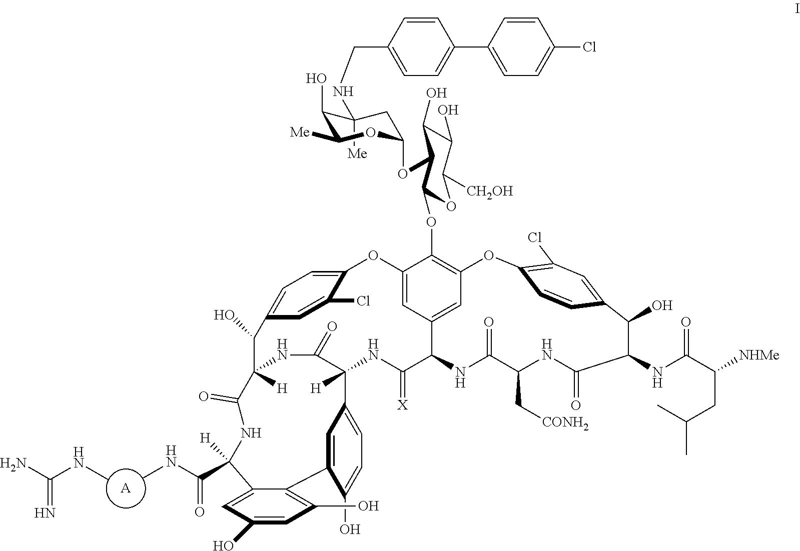

moiety that has a length of equal to greater than a saturated chain of two carbon atoms and less than a saturated chain of about 12 carbon atoms.

In examining the above structural formula, the depicted substituent bonded to the amino glycosyl moiety is referred to herein as a (4-chlorobiphenyl)-methyl group. That substituent group is usually abbreviated as a “CBP” group.

Looking further, the “X” moiety can be H,H making the carbon to which the two hydrogens are bonded a methylene group. In another embodiment, “X” is S (sulfur) double-bonded to the depicted carbon, making that carbon a thiocarbonyl moiety and thereby, the thiocarbonyl bonded to the —NH— group form a thioamide linkage. A compound where “X” is S is usually used as an intermediate to the preparation of a compound of Formula I in which “X” is NH, forming an amidine linkage. Vancomycin in which “X” is “O” (oxygen) is the native form of the compound, albeit the native compound does not include any vancosaminyl substituent, nor a C-terminal guanidinium group as shown in Formula I.

Syntheses of aglycone, glycosylated, and CPB-substituted glycosylated vancomycin derivatives are disclosed and discussed in citations 29-32 and 49-52 herein, as are compounds containing a C-terminal quaternary ammonium compound as well as the “X” group alternatives and the presence of a CPB-substituted glycosyl group. The reader's attention is also invited to U.S. Pat. Nos. 9,879,049 and 10,577,395, as well as to U.S. Patent Publication 2020/0071359 published on Mar. 5, 2020.

As noted previously, a guanidine group is quite basic and is usually present in its protonated form as a guanidinium ion, although that form is not shown in Formula I. An anion must accompany the guanidinium cation. Preferably, that anion is a pharmaceutically acceptable anion such as a halide like chloride, bromide, or iodide, a carbonate, bicarbonate, sulfate, bisulfate, benzene sulfonate, or a methane sulfonate. Appropriate anions are discussed further hereinafter. A compound of Formula I or its pharmaceutically acceptable salt is a contemplated compound of this invention.

A contemplated linker moiety Circle A is divalent and the two bonds are linked to the amido nitrogen and to the guanidino nitrogen shown in Formula I, respectively. The linker moiety Circle A is shown herein and in the claims as

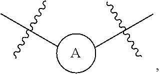

wherein the wavy lines indicate that the structure is bonded at two positions and is not itself a molecule, but a part of a molecule.

When examined along its longest chain of atoms bonded to those amido and guanidino nitrogen atoms, a linker moiety Circle A has a length that is equal to or greater than a saturated chain of two carbon atoms and less than a saturated chain of about 12 carbon atoms, and preferably about 10 carbon atoms, even though many more atoms can be present in ring structures or substituents that are not part of the chain of atoms that link those amido and guanidino nitrogen atoms. This length requirement is discussed further below.

Looked at more generally, and aside from specific moieties from which it is constructed, a particularly preferred Circle A radical (linker, group or moiety) has a length greater than about that of a three carbon chain (trimethylene group). Such a radical also has a length that is less than about that of a lauryl (dodecyl) group. That is to say that a particularly preferred Circle A is a radical having a length of or greater than about that of a saturated three carbon chain (trimethylene; —CH₂CH₂CH₂—), and shorter than about that of a saturated twelve carbon chain. More preferably, the radical has a length greater than that of a trimethylene group and less than about that of a decyl group (ten carbon chain).

The chain lengths herein are measured along the longest linear atom chain in the radical between the amido nitrogen and the guanidino nitrogen shown in Formula I, and each atom in the chain is presumed to be carbon for ease in calculation. The lengths are thus recited in terms of carbon atoms. Such lengths can be readily determined by using published bond angles, bond lengths and atomic radii, as needed, to draw and measure a staggered chain, or by building models using commercially available kits whose bond angles, lengths and atomic radii are in accord with accepted, published values.

For example, a 1,4-bonded 6-membered aromatic ring group (phenyl) not part of a fused ring system has a length of about a butyl group. A 1,2- or 1,3-bonded 6-ring has a length of a 2- or 3-carbon chain, respectively, as the shortest path around the ring between the two bonding position regardless of formal naming criteria. Where one or more 5-membered rings not part of a fused ring system are present, length is calculated as the length of a 2-carbon chain. Thus, for single ring systems, length is calculated as the shortest path around the rings between the two bonding positions to the amido and guanidino nitrogen atoms if a compound of Formula I regardless of formal naming criteria.

Similarly, in fused ring systems where two rings are directly joined together at two atoms, length is calculated as the shortest path between the two bonding positions of the divalent fused ring and a) the depicted nitrogen atoms in Formula I, above, b) one of those depicted nitrogens and another moiety within the Circle A radical, or c) between the fused ring and two other moieties within the Circle A group. The ring-to-ring bridge-head positions are not counted.

Radical lengths can also be determined somewhat less exactly by assuming that all atoms have bond lengths of saturated C—C bonds, that unsaturated bonds have the same lengths as saturated bonds, and that bond angles for unsaturated bonds are the same as those for saturated C—C bonds, although the above-mentioned modes of measurement are preferred. Both methods produce similar results within one or two carbon atoms, and thus the use of “about”.

A contemplated linker moiety Circle A can also be a hydrocarbyl chain of two to about 12 saturated carbon atoms, or preferably two to about ten saturated carbon atoms. A more preferred linking Circle A group contains a chain of atoms that is equal to or greater than the length of two saturated carbons and is shorter than about a saturated ten carbon (decyl) chain. More preferably, the radical has a chain length of two saturated carbon atoms to about eight saturated carbon (octyl) atoms. In this instance, when there is a chain of Circle A atoms linking the amido and guanidino nitrogen atoms of Formula I atoms together, the length is simply the length of the longest chain of atoms linking those two nitrogens.

In further examining Circle A hydrocarbyl linker groups, it is noted that such groups can contain one or up to four substituents that can be pendant from the chain of atoms that link the amido and guanidino nitrogens shown in Formula 1. Those substituents can be the same or different and are selected from amino acid side chain substituents other than those containing a carboxyl group, a sulfhydryl group (—SH— or a ring structure, C₁-C₃ straight chain, branched chain and cyclic hydrocarbyl groups that are saturated or unsaturated and are other than amino acid side chain substituents. Additional pendant substituents include 2-hydroxyethyl and 2-hydroxypropyl, C₁-C₃-hydrocarbyl C₀-C₂-carboxylate, and C₀-C₂-carboxamide whose amido nitrogen is unsubstituted (—NH₂), monosubstituted (—NHR¹) or disubstituted (—NR¹R²) in which the substituent (R¹ and/or R²) is one or two same or different C₁-C₄ hydrocarbyl group, or whose amido nitrogen along with two substituents together form a 5- or 6-membered hydrocarbyl ring, or a heterocyclic ring containing one additional oxygen (O) atom or a N-methyl group in the ring. In the previous sentence, “C₀” is intended to indicate that the carbonyl carbon is bonded directly to an atom of the Circle A linking chain.

A contemplated linker moiety Circle A atom chain need not be entirely hydrocarbyl, but can also be contain 1, 2, or 3 oxygens in place of carbon atoms as when a —CH₂—CH₂—O—CH₂—CH₂—, —CH₂—CH₂—O—CH₂—CH₂—O—CH₂—CH₂—, or —CH₂—CH₂—O—CH₂—CH₂—O—CH₂—CH₂—O—CH₂—CH₂— Circle A linker moiety is utilized. Similarly, the above oxygens can instead be nitrogen atoms, and preferably N-methylated nitrogens so that a contemplated preferred linker moiety Circle A has a formula —CH₂—CH₂—NCH₃—CH₂—CH₂—, —CH₂—CH₂—NCH₃—CH₂—CH₂—NCH₃—CH₂—CH₂—, or —CH₂—CH₂—NCH₃—CH₂—CH₂—NCH₃—CH₂—CH₂—NCH₃—CH₂—CH₂—.

A contemplated Circle A linker moiety also can comprise a ring system that can be carbocyclic or heterocyclic, or both as discussed below. A ring system containing one carbocyclic ring fused to a heterocyclic ring as in benzoxazole, is considered herein to be heterocyclic. Thus, a single 5- or 6-membered ring or a fused ring structure containing two rings optionally contains one or two ring hetero atoms per ring that can independently be nitrogen, oxygen or sulfur.

Individual single rings can be aliphatic or aromatic, including heteroaromatic, and also be aralkyl as in a benzyl (tolyl) group. Fused rings can both be aliphatic as in decalin, both can be aromatic as in naphthalein, or one can be aromatic and the other aliphatic as in tetralin.

A linker moiety Circle A can also be comprised of two separate rings referred to herein as Circle B and Circle C,

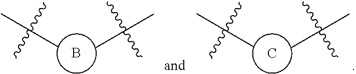

Circle B and Circle C rings can be the above-described carbocyclic or heterocyclic rings or fused rings so long as their length together as linker moiety is within the previously noted length that is shorter than about the length of a saturated twelve carbon chain that defines a Circle A group. Circle B and Circle C rings can be the same or can be different. When Circle B and Circle C rings are the same, they are nonetheless referred to as Circle B and Circle C rings for clarity.

The Circle B and Circle C rings can be bonded together directly as in a biphenyl group, or they can be bonded indirectly together by a divalent sub-linker group Z that contains one, two, three or four atoms in the chain of atoms used for Circle A linker length determination. Alternatively, one or the other of Circle B or Circle C can be bonded via the divalent sub-linker Z to one or the other of the amido nitrogen or the guanidino nitrogen shown in Formula I.

In another embodiment, a plurality of divalent sub-linker Z groups can be present as a part of a Circle A linker. In this embodiment, two or more divalent sub-linker Z groups can be present along with one or more Circle B and Circle C rings. When more than one divalent sub-linker Z group is present, the divalent sub-linker Z groups can be the same or different. When two or more of the same divalent sub-linker Z groups are present, they are labeled by the same subscripted numeral, e.g., Z₁, and different Z groups are labeled with different subscripted numerals, e.g., Z₂, Z₃, etc.

A contemplated divalent sub-linker group Z includes the following: —O—, —S—, —NH—, —CO—NH—, —NH—CO—, —CO—O—, —O—CO—, —O—CO—O—, —C≡C—, —HC═CH—, —H₂C—CH₂—, —CH₂—, —NH—CO—NH—, —NH—CO—O—, —O—CO—NH—, —N═N—, —NH—NH—, —CS—NH—, —HN—CS—, —SO— and —SO₂—. It is noted that a carbonyl oxygen, a sulfinyl oxygen, the sulfonyl oxygens and a thiocarbonyl sulfur are not included in the atom chain utilized for determining the length of a Circle A linker moiety.

Using monovalent substituent names for convenience, exemplary aromatic carbocyclic ring moieties include phenyl and naphthyl groups. Again, using monovalent names for convenience, exemplary heteroaryl groups include 6-membered ring substituents such as pyridyl, pyrazyl, pyrimidinyl, and pyridazinyl; 5-membered ring substituents such as 1,3,5-, 1,2,4- or 1,2,3-triazinyl, imidazyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl groups; 6/5-membered fused ring substituents such as benzothiofuranyl, isobenzothiofuranyl, benzisoxazolyl, benzoxazolyl, purinyl and anthranilyl groups; and 6/6-membered fused rings such as 1,2-, 1,4-, 2,3- and 2,1-benzopyronyl, quinolinyl, isoquinolinyl, quinoxalinyl, cinnolinyl, quinazolinyl, and 1,4-benzoxazinyl groups.

Aliphatic 5- and 6-membered carbocyclic rings are contemplated such as decalin, cyclohexyl and cyclopentyl, as well as their mono- and diethylenically unsaturated derivatives, using monovalent names for convenience. Again using monovalent radical names for convenience, aliphatic 5- and 6-membered heterocyclic rings include, piperidinyl, piperazinyl, imidazolinyl, imidazolidinyl, pyrrolinyl, pyrrolidinyl, pyrazolidinyl, pyrazolinyl, pyranyl, morpholinyl, oxazinyl, isooxazinyl, and oxathiolyl.

Aliphatic/aromatic fused rings include, using monovalent nomenclature, isochromanyl, chromanyl, chromenyl, 3H-indolyl, and tetralin.

Still further 5- and 6-membered single rings and fused ring groups that are aliphatic (including heterocyclic) or aromatic (including heteroaromatic) and 5/5-, 5/6- and 6/6-fused ring compounds can be found in Handbook of Chemistry and Physics, 54^{th} Ed., R. C. Weast, Ph.D. ed., CRC Press, 1973-1974, pp. C-1-C73.

The present invention is exemplified in part by the illustrative listing of Circle A linker moieties shown below:

### Illustrative Circle a Linker Moieties

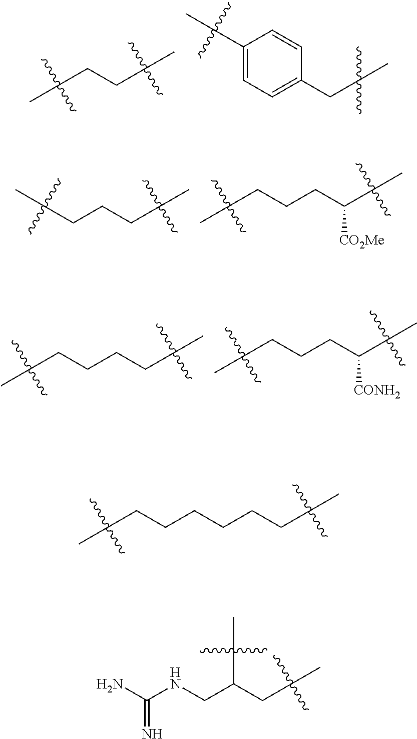

Structural formulas of preferred pharmaceutical compounds of Formulas Ia, Ib and Id are shown below:

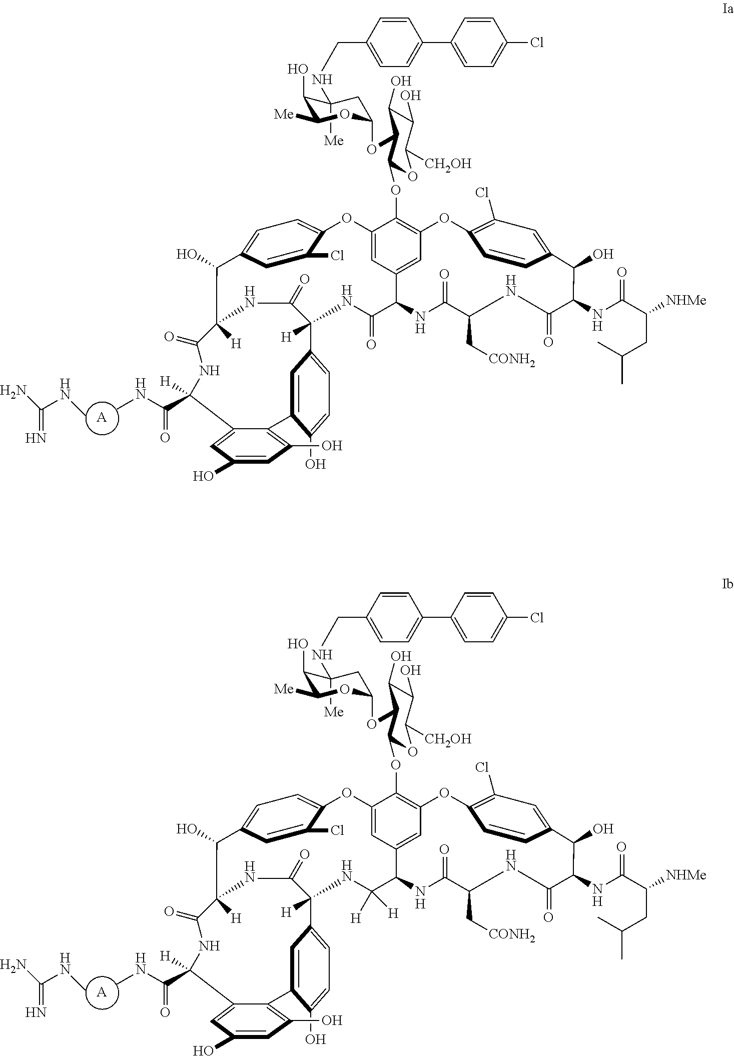

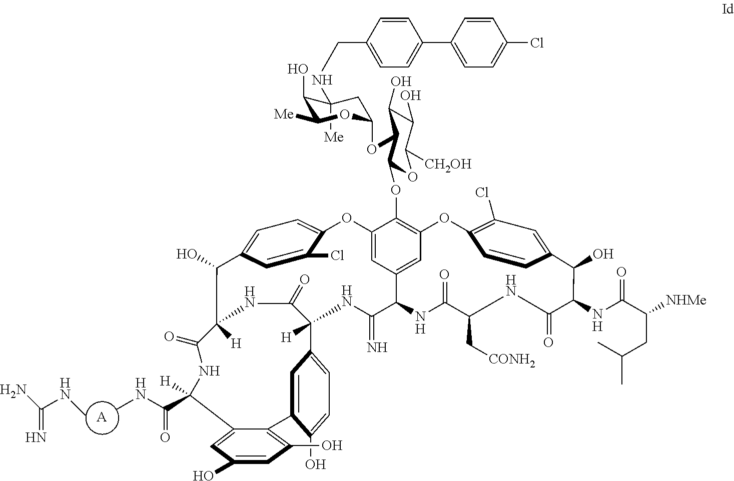

Preferred individual compounds of the above compounds contain Circle A linker moieties that have a length of a saturated chain of 2 carbon atoms to a saturated chain of about 10 carbon atoms, and more preferably, that chain length is 2 to about 8 saturated carbon atoms.

A compound of structural Formula Ic is also particularly preferred as it is a precursor intermediate to the preparation of a compound of Formula Id

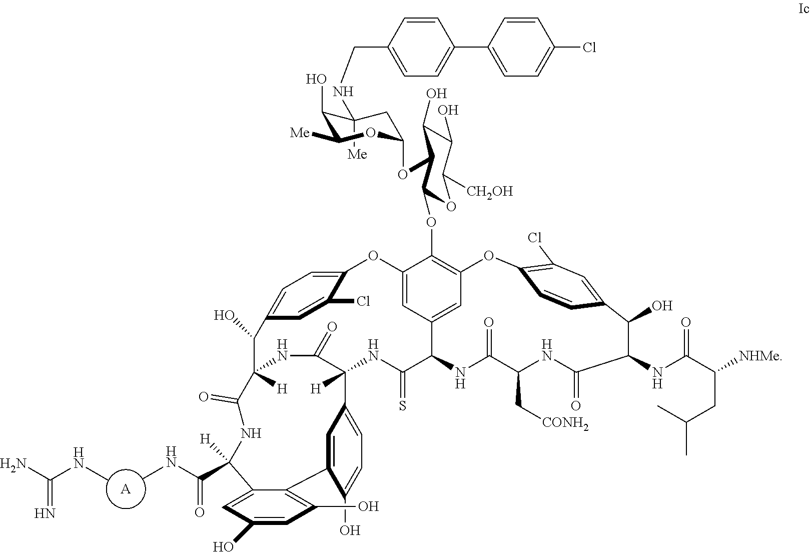

### Composition and Treatment Method

A further aspect of the invention is a method of treating a mammal infected with a microbial infection such as a bacterial infection, typically either a Gram-positive infection or a Gram-negative bacterium; i.e., an infection caused by Gram-positive or Gram-negative bacteria, and in need of antimicrobial (antibacterial) treatment. In accordance with a contemplated method, an antibacterial-effective amount of one or more compounds of Formula I or a pharmaceutically acceptable salt of such a compound is administered to an infected mammal in need.

The compound can be administered as a solid or as a liquid formulation, and is preferably administered via a pharmaceutical composition discussed hereinafter. That administration can also be oral or parenteral, as are also discussed further hereinafter.

It is to be understood that mammals are infected with bacteria and other microbes. The present invention's method of treatment is intended for use against infections of pathogenic bacteria that cause illness in the mammal to be treated. Illustrative pathogenic microbes include S. aureus, methicilin-resistant S. aureus (MRSA), VanA strains of E. faecalis and E. faecium, as well as VanB strains of E. faecalis. Evidence of the presence of infection by pathogenic microbes is typically understood by physicians and other skilled medical workers.

A mammal in need of treatment (a subject) and to which a pharmaceutical composition containing a Compound of Formula I or its pharmaceutically acceptable salt can be administered can be a primate such as a human, an ape such as a chimpanzee or gorilla, a monkey such as a cynomolgus monkey or a macaque, a laboratory animal such as a rat, mouse or rabbit, a companion animal such as a dog, cat, horse, or a food animal such as a cow or steer, sheep, lamb, pig, goat, llama or the like.

As is seen from the data that follow, a contemplated compound is active in in vitro assay studies at less than 1 μg/mL amounts, which corresponds to a molar concentration of about 1 to about 100 nanomolar (nM), using the molecular weight of Compound 15. When used in an assay such as an in vitro assay, a contemplated compound is typically present in the composition in an amount that is sufficient to provide a concentration of about 0.1 nM to about 1 μM to contact microbes to be assayed.

The amount of a compound of Formula I or a pharmaceutically acceptable salt of such a compound that is administered to a mammal in a before-discussed method or that is present in a pharmaceutical composition discussed below, which can be used for that administration, is an antibiotic (or antibacterial or antimicrobial) effective amount. It is to be understood that that amount is not an amount that is effective to kill all of the pathogenic bacteria or other microbes present in an infected mammal in one administration. Rather, that amount is effective to kill some of the pathogenic organisms present without also killing the mammal to which it is administered, or otherwise harming the recipient mammal as is well known in the art. As a consequence, the compound usually has to be administered a plurality of times, as is discussed in more detail hereinafter.

A contemplated pharmaceutical composition contains an effective antibiotic (or antimicrobial) amount of a Compound of Formula I or a pharmaceutically acceptable salt thereof dissolved or dispersed in a physiologically (pharmaceutically) acceptable diluent or carrier. An effective antibiotic amount depends on several factors as is well known in the art. However, based upon the relative potency of a contemplated compound relative to that of vancomycin itself for a susceptible strain of S. aureus shown hereinafter, and the relative potencies of vancomycin and a contemplated compound against the VanA E. faecalis and E. faecium strains, a skilled worker can readily determine an appropriate dosage amount.

Exemplary salts useful for a contemplated compound include but are not limited to the following: sulfate, hydrochloride, hydro bromides, acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxy-ethanesulfonate, lactate, maleate, methanesulfonate, nicotinate, 2-naphthalenesulfonate, oxalate, palmoate, pectinate, persulfate, 3-phenyl-propionate, picrate, pivalate, propionate, succinate, tartrate, thiocyanate, tosylate, mesylate and undecanoate.

The reader is directed to Berge, J. Pharm. Sci. 1977 68(1):1-19 for lists of commonly used pharmaceutically acceptable acids and bases that form pharmaceutically acceptable salts with pharmaceutical compounds.

In some cases, the salts can also be used as an aid in the isolation, purification or resolution of the compounds of this invention. In such uses, the salt prepared need not be pharmaceutically acceptable.

A contemplated composition is typically administered repeatedly in vivo to a mammal in need thereof until the infection is diminished to a desired extent, such as cannot be detected. Thus, the administration to a mammal in need can occur a plurality of times within one day, daily, weekly, monthly or over a period of several months to several years as directed by the treating physician. More usually, a contemplated composition is administered a plurality of times over a course of treatment until a desired effect is achieved, typically until the bacterial infection to be treated has ceased to be evident.

A contemplated pharmaceutical composition can be administered orally (perorally) or parenterally, in a formulation containing conventional nontoxic pharmaceutically acceptable carriers or diluents, adjuvants, and vehicles as desired. The term parenteral as used herein includes subcutaneous injections, intravenous, intramuscular, intrasternal injection, or infusion techniques. Formulation of drugs is discussed in, for example, Hoover, John E., Remington's Pharmaceutical Sciences, Mack Publishing Co., Easton, Pa.; 1975 and Liberman, H. A. and Lachman, L., Eds., Pharmaceutical Dosage Forms, Marcel Decker, New York, N.Y., 1980.

In some embodiments, a contemplated pharmaceutical composition is preferably adapted for parenteral administration. Thus, a pharmaceutical composition is preferably in liquid form when administered, and most preferably, the liquid is an aqueous liquid, although other liquids are contemplated as discussed below, and a presently most preferred composition is an injectable preparation.

Thus, injectable preparations, for example, sterile injectable aqueous or oleaginous solutions or suspensions can be formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be employed are water, Ringer's solution, isotonic sodium chloride solution, and phosphate-buffered saline.

Other liquid pharmaceutical compositions include, for example, solutions suitable for parenteral administration. Sterile water solutions of a Compound of Formula I or its salt or sterile solution of a Compound of Formula I in a solvent comprising water, ethanol, or propylene glycol are examples of liquid compositions suitable for parenteral administration. In some aspects, a contemplated Compound of Formula I is provided as a dry powder that is to be dissolved in an appropriate liquid medium such as sodium chloride for injection prior to use.

In addition, sterile, fixed oils are conventionally employed as a solvent or suspending medium. For this purpose any bland fixed oil can be employed including synthetic mono- or diglycerides. In addition, fatty acids such as oleic acid find use in the preparation of an injectable composition. Dimethyl acetamide, surfactants including ionic and non-ionic detergents, polyethylene glycols can be used. Mixtures of solvents and wetting agents such as those discussed above are also useful.

A sterile solution can be prepared by dissolving the active component in the desired solvent system, and then passing the resulting solution through a membrane filter to sterilize it or, alternatively, by dissolving the sterile compound in a previously sterilized solvent under sterile conditions.

Solid dosage forms for oral administration can include capsules, tablets, pills, powders, and granules. The amount of a contemplated Compound or salt of Formula I such as Compound 15 in a solid dosage form is as discussed previously, an amount sufficient to provide an effective antibiotic (or antimicrobial) amount. A solid dosage form can also be administered a plurality of times during a one week time period.

In such solid dosage forms, a compound of this invention is ordinarily admixed as a solution or suspension in one or more diluents appropriate to the indicated route of administration. If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, magnesium or calcium carbonate or bicarbonate. Tablets and pills can additionally be prepared with enteric coatings.

Where an in vitro assay is contemplated, a sample to be assayed such as cells and tissue can be used. These in vitro compositions typically contain water, sodium or potassium chloride, and one or more buffer salts such as and acetate and phosphate salts, Hepes or the like, a metal ion chelator such as EDTA that are buffered to a desired pH value such as pH 4.0-8.5, preferably about pH 7.2-7.4, depending on the assay to be performed, as is well known.

Preferably, the pharmaceutical composition is in unit dosage form. In such form, the composition is divided into unit doses containing appropriate quantities of the active compound. The unit dosage form can be a packaged preparation, the package containing discrete quantities of the preparation, for example, in vials or ampules.

Results and Discussion

### C-Terminus Guanidine Modifications

A G3 unit (a guanidinium-containing amine bearing a C3 linker) was chosen initially for introduction at the vancomycin C-terminus by amide coupling, resulting in the guanidine-modified vancomycin analogue Compound 5 (Scheme 1). It is nearly identical to C1-vancomycin (Compound 3) disclosed in our previous reports²⁹ with the only difference being the replacement of the trimethylammonium cation (C1) with a guanidine group at the C-terminus of the glycopeptide.

The antimicrobial activity of Compound 5 against vancomycin-resistant organisms (VanA VRE, 4 strains) was evaluated in a standard microdilution assay. It was found that the introduction of this small and simple peripheral modification in Compound 5 resulted in a potent antimicrobial activity against VanA VRE (MIC=4 to 16 μg/mL), representing not only a substantial improvement in antimicrobial potency compared with vancomycin (8 to 64-fold), but that it also proved more potent than C1-vancomycin (Compound 3, 4-fold) disclosed in our previous reports.

The structure-activity relationships (SAR) of the guanidine modification were explored to establish the source of the outstanding activity of Compound 5. A variety of guanidine-containing modifications with varied linker lengths (Compounds 6-8) and linker rigidity (Compound 9) were selected. Compounds 10-12 that can be viewed as derivatives of Compound 7 were also prepared with an added side chain substituent.

In order to demonstrate the effect of the positive charge of the guanidinium group, Compound 13 was also prepared where the positively charged guanidinium group in Compound 10 was replaced by a neutral urea group still capable of hydrogen bonding. Finally, an amine bearing two guanidine groups (Compound 14) was attached to vancomycin to examine the influence of an additional positive charge to the properties of the candidate antibiotic.

Notably, Compound 5 and the subsequent analogues Compounds 6-14 were prepared by direct amide bond coupling of the corresponding guanidine-containing amine with vancomycin without the introduction or removal of intermediate protecting groups. The antimicrobial activity of these analogues against vancomycin-resistant as well as vancomycin-sensitive organisms was determined.

The Compounds 6-9 maintained the superb antimicrobial potency of Compound 5 against the four VanA VRE strains tested (Table 1), which indicates that the improved activity is insensitive to the linker length and rigidity within the small range examined. The examination of Compounds 10 and 12 revealed that the improved activity is not affected by the presence of charge-neutral linker substituents.

However, Compound 13 that replaced the protonated guanidinium group with a neutral urea group exhibited reduced activity against resistant strains (compared with Compound 10). Interestingly and although not initially expected, the presence of an extra negatively charged side chain carboxylic acid led to a significant decrease in antimicrobial potency (8 to 32-fold) of the analogue (Compound 11).

These observations demonstrate not only the importance of the protonated guanidinium positive charge, but also that the net positive charge change on the C-terminus is essential to the outstanding activity exhibited by these guanidine analogues⁴⁰. It is also noteworthy that a 2 to 4-fold enhancement in antimicrobial potency against sensitive strains (S. aureus) was also observed for most of the guanidinium-containing vancomycin analogues beyond the already excellent activity of vancomycin itself. It is note that Wender and coworkers independently have recently disclosed compound 12 as a simplification of a vancomycin-octaarginine conjugate (V-r8)⁷⁰ with incorporation of single arginine (V-r1)⁷¹ and that it provided a vancomycin analogue which displayed activity against Gram-negative bacteria and maintained the activity of vancomycin against Gram-positive organisms.

TABLE 1
Antimicrobial activity of Compounds 1 and 5-14
against four VanA VRE strains, MIC (μg/mL).^{a}

| | | VanA | VanA | |
|---|---|---|---|---|
| | VanA | E. faecium | E. faecalis | VanA |
| | E. faecalis | (ATCC | (ATCC | E. faecium |
| Compound | (BM 4166) | BAA-2317} | BAA-2573) | (TX2465) |
| vancomycin (1) | 250 | 250 | 125 | 250 |
| G3-vancomycin (5) | 16 | 4 | 4 | 16 |
| G2-vancomycin (6) | 16 | 4 | 8 | 16 |
| G4-vancomycin (7) | 16 | 4 | 4 | 16 |
| G6-vancomycin (8) | 16 | 4 | 4 | 16 |
| GBn-vancomycin (9) | 16 | 4 | 4 | 8 |
| Arg(OMe)-vancomycin (10) | 8 | 8 | 4 | 8 |
| Arg-vancomycin (11) | 63 | 63 | 125 | 125 |
| Arg(NH₂)-vancomycin (12) | 16 | 4 | 4 | 8 |
| Cit(OMe)-vancomycin (13) | 63 | 31 | 16 | 63 |
| DiG-vancomycin (14) | 8 | 1 | 2 | 4 |

^{a}MIC = minimum inhibitory concentration.

The effect of the incorporation of the guanidinium modifications into CBP-vancomycin (Compound 2) was subsequently examined. By virtue of the incorporation of the CBP group, CBP-vancomycin (Compound 2) already possess one effective mechanism of action independent of D-Ala-D-Ala/D-Ala-D-Lac binding against vancomycin-resistant strains (transglycosylase inhibition)³⁴⁻³⁵.

The guanidinium-containing CBP-vancomycin analogues were prepared from Compound 2 in a single and scalable step by direct amide coupling of the corresponding guanidinium-containing amine without intermediate protection or deprotection (Scheme 1). To our delight, the incorporation of guanidine group at the C-terminus of Compound 2 further enhanced the antimicrobial potency of the analogues (2 to 8-fold) against the four VanA VRE strains tested (Table 2).

The net positive charge at the C-terminus was found again to be crucial to the antimicrobial potency, where Compounds 21 and 23 exhibited no enhanced antimicrobial potency. Incorporation of two guanidine groups (Compound 24) offered no further improvement.

Both G3-CBP-vancomycin (V15) and GBn-CBP-vancomycin (Compound 19) emerged as representative of the most effective compounds in the series. Notable is the superb sub-μg/mL activity of the analogues (0.6-0.15 μg/mL) against the small panel of VanA VRE that is derived from two peripheral modifications providing two independent and synergistic mechanisms of action, neither of which directly impact D-Ala-D-Ala/D-Ala-D-Lac binding. Combined, the CBP and guanidine modifications increased the activity against VanA VRE as much as 1000-fold relative to vancomycin itself.

TABLE 2
Antimicrobial activity of Compounds 1, 2 and
15-24 against VanA VRE strains, MIC (μg/mL)

| | | VanA | VanA | |
|---|---|---|---|---|
| | VanA | E. faecium | E. faecalis | VanA |
| | E. faecalis | ATCC | ATCC | E. faecalis |
| Compound | BM 4166 | BAA-2317 | BAA-2573 | TX 2465 |
| vancomycin (1) | 250 | 250 | 125 | 250 |
| CBP-vancomycin (2) | 5 | 2.5 | 0.3 | 5 |
| G3-CBP-vancomycin (15) | 0.6 | 0.3 | 0.15 | 0.6 |
| G2-CBP-vancomycin (16) | 0.6 | 0.3 | 0.15 | 1.2 |
| G4-CBP-vancomycin (17) | 0.6 | 0.3 | 0.15 | 0.6 |
| G6-CBP-vancomycin (18) | 0.6 | 0.3 | 0.3 | 1.2 |
| GBn-CBP-vancomycin (19) | 0.6 | 0.3 | 0.15 | 0.6 |
| Arg(OMe)-CBP-vancomycin (20) | 0.6 | 0.3 | 0.15 | 0.6 |
| Arg-CBP-vancomycin (21) | 1.2 | 1.2 | 0.15 | 2.5 |
| Arg(NH₂)-CBP-vancomycin (22) | 0.6 | 0.3 | 0.15 | 0.6 |
| Cit(OMe)-CBP-vancomycin (23) | 1.2 | 2.5 | 0.3 | 5 |
| DiG-CBP-vancomycin (24) | 0.6 | 0.6 | 0.15 | 0.3 |

Just as significantly and as highlighted earlier, the guanidine-containing modifications also increased the antimicrobial activity of both vancomycin or CBP-vancomycin against sensitive bacteria strains by 2 to 4-fold, albeit being less pronounced because of the already superb activity of vancomycin or CBP-vancomycin. This is illustrated with both a sensitive and methicillin-resistant S. aureus strain and an insensitive VanB VRE strain in Table 3, below.

TABLE 3
Antimicrobial activity of 1, 2, 5, 9, 15 and 19 against
VanB and S. aureus strains, MIC (μg/mL).

| | VanB | Sensitive | MRSA |
|---|---|---|---|
| | E. faecalis | S. aureus | S. aureus |
| | ATCC | ATCC | ATCC |
| Compound | 51299 | 25923 | 43300 |
| vancomycin (1) | 8 | 0.5 | 0.5 |
| G3-vancomycin (5) | 8 | 0.25 | 0.12 |
| GBn-vancomycin (9) | 8 | 0.25 | 0.12 |
| CBP-vancomycin (2) | 0.08 | 0.08 | 0.08 |
| G3-CBP-vancomycin (15) | 0.04 | 0.04 | 0.02 |
| GBn-CBP-vancomycin (19) | 0.04 | 0.04 | 0.02 |

### Site Specific Nature of the Guanidine Modification

Analogous to observations made in our studies with the trimethylammonium cations³⁷, the introduction of the guanidinium group as an A-ring substituent versus at the C-terminus provided less active analogues, highlighting the site selectivity of the impact of the modification. Similarly, a C-terminus and A-ring doubly modified guanidinium analogue failed to improve on the activity of the corresponding single C-terminus modification. Finally, and like the C-terminus trend, an A-ring guanidinium modification did prove to be more potent than a corresponding A-ring trimethylammonium cation (C1) analogue.

### Activity Against Gram-Negative Bacteria.

As noted earlier, Wender and coworkers independently disclosed compound 12 as a simplification of a vancomycin-octaarginine conjugate (V-r8)⁷⁰ with incorporation of single arginine (V-r1)⁷¹ and that it provided a vancomycin analogue that displayed activity against Gram-negative bacteria. The generalizability of this observation was investigated with the C-terminus guanidine modified vancomycins (5 and 9) and CBP-vancomycins (15 and 19) and their activity against a small panel of Gram-negative bacteria. Reduced strength broth conditions were required to observe the activity of these analogues, but the C-terminus guanidine modifications in 5 and 9 conveyed potent activity against a small panel of Gram-negative bacteria, increasing activity by 4 to 32-fold and with some MICs as low as 1-2 mg/mL. It is noteworthy that although the CBP modification provided an additional mechanism of action and improved activity against Gram-positive bacteria, it was found to reduce this activity of 5 and 9 against Gram-negative bacteria in the small series of compounds examined.

### G3-CBP-Vancomycin is a Durable Antibiotic

Vancomycin analogues with multiple synergistic mechanisms of action not only exhibit superb potency against vancomycin-resistant organisms, they also display an even more important enhanced durability where the rate of emergence of bacterial resistance significantly decreased with each added mechanism of action²⁹. Such durable antibiotics expressing multiple mechanisms of action are expected to suppress the emergence of bacterial resistance even with extensive use.

In addition to the importance of demonstrating the enhanced durability, such comparative studies also provide indirect evidence of an added mechanism of action. Thus, in order to establish whether the guanidine-modified vancomycin analogues express an added antimicrobial mechanism of action, a multi-passage resistance development assay was conducted initially for 25 days, but then extended to 50 days to further illustrate the superb durability of these compounds.

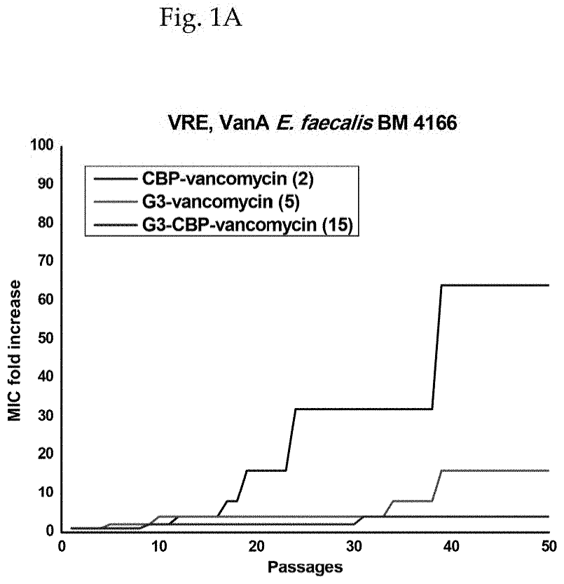

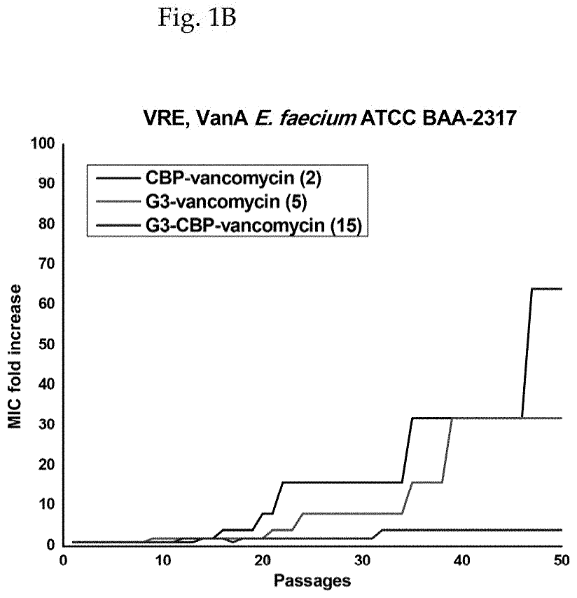

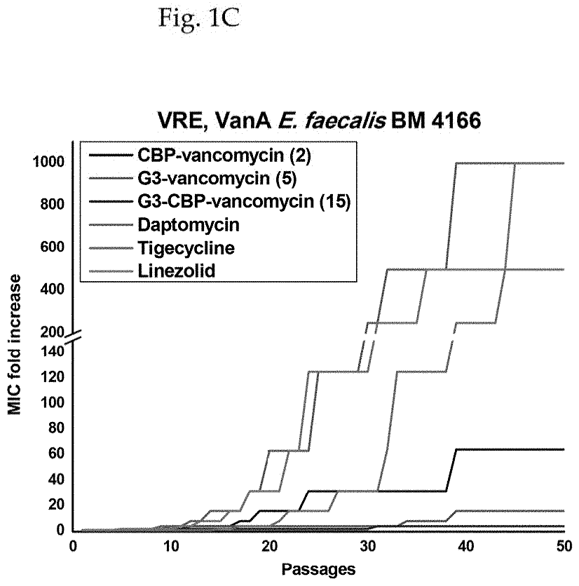

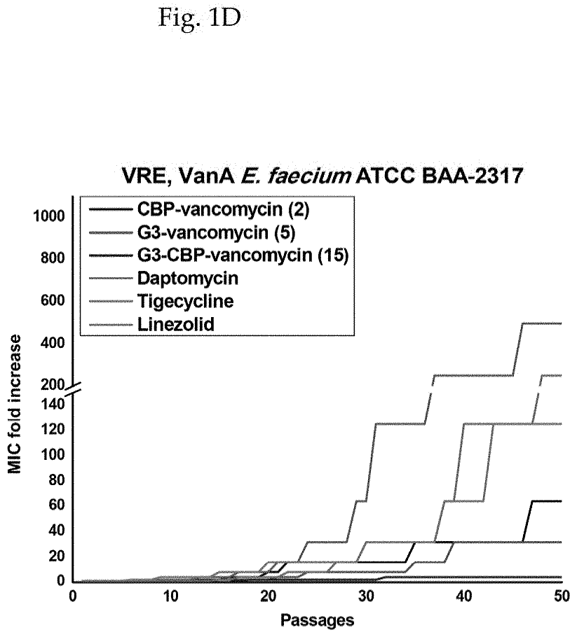

This study was conducted with two VanA VRE strains already resistant to vancomycin (MIC=250 μg/mL), notably no longer susceptible to its cell wall biosynthesis blockage through D-Ala-D-Ala binding and transpeptidase inhibition (FIGS. 1A-1D). Thus, the studies were not only conducted over a longer period than is typical (50 vs 25 days), but were also conducted with vancomycin-resistant organisms that overcome the intrinsic mechanism of action of vancomycin (D-Ala-D-Ala binding) and that are further resistant to many other antibiotic classes⁴¹ by virtue of assimilation of additional common mechanisms of resistance. As such, they are on the verge of becoming multidrug and vancomycin-resistant organisms.

G3-Vancomycin (Compound 5) exhibited a better durability (4 to 8-fold increase in MIC, 1 MOA) than CBP-vancomycin (Compound 2, 16 to 32-fold increase in MIC, 1 MOA) over 25 days. G3-CBP-vancomycin (Compound 15), the analogue bearing the two peripheral modifications, exhibited a further enhanced durability (2-fold increase in MIC). Even with extension of the study to 50 days, only a subtle 4-fold increase in MIC was observed with G3-CBP-vancomycin (Compound 15), and the antibiotic remains potent (MIC=1.2-2.5 μg/mL) against the two VanA VRE strains at the end of the study.

In contrast, the analogue Compounds 2 and 5 with single modifications have lost their activity and display MIC values similar to vancomycin by the end of resistance development assay. Moreover, and with these two VanA VRE strains, the frontline therapies daptomycin, linezolid, and tigecycline raise resistance more rapidly and in a more pronounced manner than either of Compounds 2 or 5²⁹, further highlighting the remarkable behavior of Compound 15.

These results demonstrate clearly the superb durability of Compound 15, the important consequence of combining the two peripheral modifications as compared with derivatives bearing only a single modification (Compounds 2 and 5), and illustrate that each peripheral modification and independent mechanism of action are important in such improvements in durability. Thus, emergence of resistance against one mechanism of action is suppressed by the other when two synergistic mechanisms of action are combined. A similar result was observed in previous work, where C1-CBP-vancomycin (Compound 4) exhibited an enhanced durability compared with CBP-vancomycin (Compound 2) due to an added mechanism of action provided by the newly introduced trimethylammonium cation.

However, the extended durability of G3-CBP-vancomycin (Compound 15) described herein exceeds that of C1-CBP-vancomycin (Compound 4), that is attributed to the more potent and robust activity derived from the G3 versus C1 modification. Combined with results of their antimicrobial activity, these studies confirm that an added mechanism of action is introduced by incorporation of the guanidinium group and that G3-CBP-vancomycin (Compound 15) and related compounds exhibit their activity through two synergistic and independent mechanisms of action against vancomycin-resistant organisms. Both of those mechanisms are independent of D-Ala-D-Ala/D-Lac binding.

### The Guanidine Modification Induces Bacteria Cell Permeabilization: An Added Synergistic Mechanism of Action.

The previously reported C-terminus trimethylammonium cation (Compound C1) modification on vancomycin induces bacteria cell permeabilization as well as the enhancement of antimicrobial activity²⁹, as indicated by propidium iodide (PI) influx. Although the permeabilization effect was found to be weaker with C1-vancomycin (Compound 3) even at high concentration (100 μM), C1-CBP-vancomycin (Compound 4), bearing both peripheral modifications, was able to induce pronounced membrane permeabilization. This observation suggests a synergistic nature of the two peripheral modifications, where the presence of one modification (CBP) enhances the effect of the other (C1).

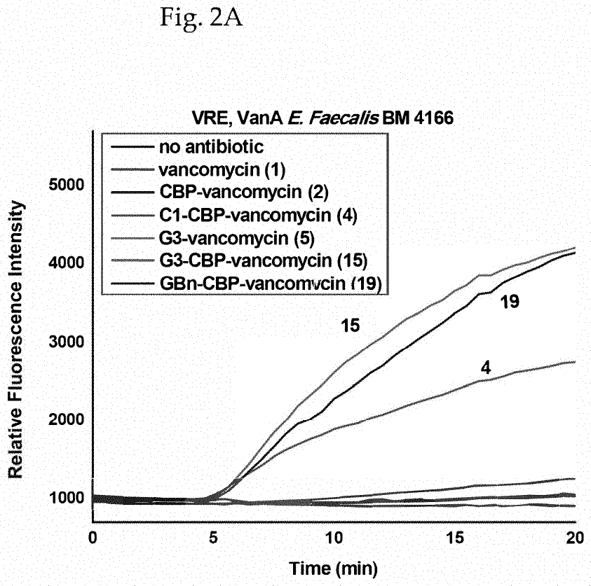

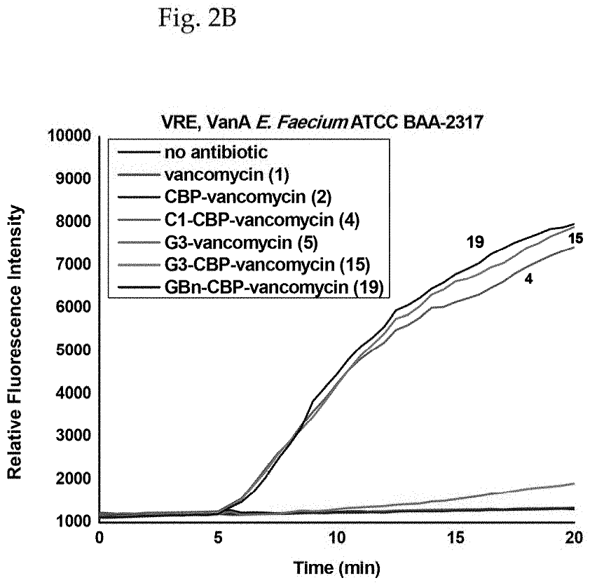

Also noted was that this inducement of bacterial membrane permeabilization and the resulting enhancement in antimicrobial activity is both structure (C1 vs other trialkylammonium salt) and site (C-terminus of vancomycin vs N-terminus or A-ring) specific³⁶⁻³⁷. In order to establish whether a similar mechanism of action is introduced by the peripheral guanidine modification, representative members of the guanidine-containing vancomycin analogues were examined in a permeability assay and conducted alongside Compounds 1, 2 and 4 (FIGS. 2A and 2B).

Although no permeabilization was induced by either vancomycin itself or CBP-vancomycin, weak permeabilization was observed upon addition of G3-vancomycin (Compound 5) at an elevated concentration (50 μM), which is accordance with our observations with C1-vancomycin. G3-CBP-vancomycin (Compound 15) and GBn-CBP-vancomycin (Compound 19) induced strong membrane permeabilization that is comparable or stronger than that observed with C1-CBP-vancomycin (Compound 4).

A reduced bacteria cell permeabilization was observed with analogues exhibiting weaker antimicrobial potency (Compounds 21 and 23), where both the initial rate and final extent of the fluorescence signal was reduced. These observations reveal that guanidine-containing vancomycin analogues induce bacterial cell permeabilization, and that the intensity of the signal in the assay directly correlates with the antimicrobial activity of such analogues. Combined with the observations made in the durability assay and similar to the observations made with the trimethylammonium cation vancomycin analogues, the C-terminus incorporation of a guanidine into vancomycin and CBP-vancomycin introduced an additional mechanism of action, resulting in membrane permeabilization independent of D-Ala-D-Ala/D-Lac binding and transglycosylase inhibition.

### Exogeneous Lipoteichoic Acid Reduces Antimicrobial Activity and Blocks Induced Bacteria Cell Permeability

A guanidinium group, serving as both a persistent positive charge under physiological conditions and a multiple hydrogen bond-donor, might well be expected to interact with negatively charged phosphate or carboxylate containing components in the bacteria cell envelope. These components are among the possible specific or non-specific binding partners for the guanidinium-containing vancomycin analogues.

During the investigation of candidate targets of the earlier trimethylammonium cation modification, it was observed that cell envelope teichoic acid (TA) behaved as a potential binding partner³⁸. Teichoic acids are major constituents found in the cell envelope of Gram-positive bacteria either in the form of lipoteichoic acids (LTA) that are anchored in the cell membrane and extend into or through the peptidoglycan cell wall⁴², or cell wall teichoic acids (WTA) that are covalently linked to N-acetylmuramic acid in the cell wall peptidoglycan^{43,42}.

These polyanionic alditol phosphate-containing polymers contribute to cell envelope stability and rigidity, cation (e.g., Mg²⁺) homeostasis and transport, and are responsible for binding, sequestration, and regulation of autolysins that act to degrade the bacteria cell wall in response to the need for damage repair or replication expansion⁴²⁻⁴⁴. To establish whether such TAs can also serve as binding partners for the guanidinium-containing vancomycin analogues like cationic peptide antibiotics^{45,46} that in turn may competitively displace sequestered autolysins, a microdilution antimicrobial assay was conducted in the presence of added LTA (100 and 1000 μg/mL) as a competitive surrogate for both LTA and WTA (Table 4, below).

TABLE 4
Impact of added lipoteichoic acid (LTA), phosphate and phospholipid (POPE)
on the antimicrobial activity of vancomycin derivatives, MIC (μg/mL).^{a}

| | VanA VRE | | VanA VRE | | |
|---|---|---|---|---|---|
| | E. faecalis | | E. faecium | | |
| | (BM 4166) | | (ATCC BAA2317) | | |
| Condition | −LTA | +LTA | −LTA | +LTA | |
| vancomycin (1) | 250 | 250 | 250 | 250 | |
| CBP-vancomycin (2) | 5 | 10 | 2.5 | 5 | |
| G3-vancomycin (5) | 16 | 16 | 4 | 8 | (63)^{b} |
| G3-CBP-vancomycin (15) | 0.6 | 5 | 0.3 | 2.5 | (10)^{b} |

| GBn-vancomycin (9) | 16 | 16 | 4 | 4 |
|---|---|---|---|---|
| GBn-CBP-vancomycin (19) | 0.6 | 5 | 0.3 | 2.5 |
| CBP-vancomycin (2) + G3⁺ | 5 | 10 | 2.5 | 5 |
| G3-CBP-vancomycin (15) + G3⁺ | 0.6 | 5 | 0.3 | 2.5 |
| G3-CBP-vancomycin (15) + Mg²⁺ | 0.6 | 5 | 0.3 | 2.5 |
| Condition | −NaH₂PO₄ | +NaH₂PO₄ | −NaH₂PO₄ | +NaH₂PO₄ |
| G3-vancomycin (5) | 16 | 16 | 4 | 4 |
| G3-CBP-vancomycin (15) | 0.6 | 0.6 | 0.3 | 0.3 |
| Condition | −POPE | +POPE | −POPE | +POPE |
| G3-vancomycin (5) | 16 | 16 | 4 | 4 |
| G3-CBP-vancomycin (15) | 0.6 | 0.6 | 0.3 | 0.3 |
| G3⁺ | >800 | >800 | | |

^{a}Assays run in the absence or presence of additives (lipoteichoic acid (LTA), NaH₂PO₄, or POPE, 100 μg/mL) as well as the absence or presence of H₂N(CH₂)₃NHC(═NH₂)NH₂⁺ (G3⁺, 100 μg/mL) or Mg²⁺ (MgCl₂, 100 μg/mL).
^{b}With 1000 μg/mL LTA.

Whereas the activity of vancomycin (no difference) and CBP-vancomycin (decrease by 2-fold) are insensitive to the added LTA, the antimicrobial activity of both Compounds 15 and 19 decreased 8-fold (100 μg/mL LTA). Because of its 10-fold higher MIC and the larger amounts of compound needed for bacterial cell growth inhibition, Compound 5 displayed the same behavior but required a compensating larger amount of added LTA (1000 μg/mL).

This impact of LTA concentration on blocking the antimicrobial activity of Compounds 5 and 15 was studied with one VanA VRE E. faecium strain (ATCC BAA-2317) and the results are summarized in Table 5, below.

TABLE 5
Antimicrobial activity of Compounds 5 and 15 in the
presence of exogeneous LTA (10, 100 or 1000 μg/mL).

Strain
VanA VRE
E. faecium
ATCC BAA-2317
Compound

| | | |
|---|---|---|
| LTA | G3- | G3-CBP- |
| concentration | vancomycin (5) | vancomycin (15) |

| | |
|---|---|
| (μg/mL) | MIC (μg/mL) |

| | | |
|---|---|---|
| 0 | 4 | 0.3 |
| 10 | 4 | 0.6 |
| 100 | 8 | 2.5 |
| 1000 | 63 | 10 |

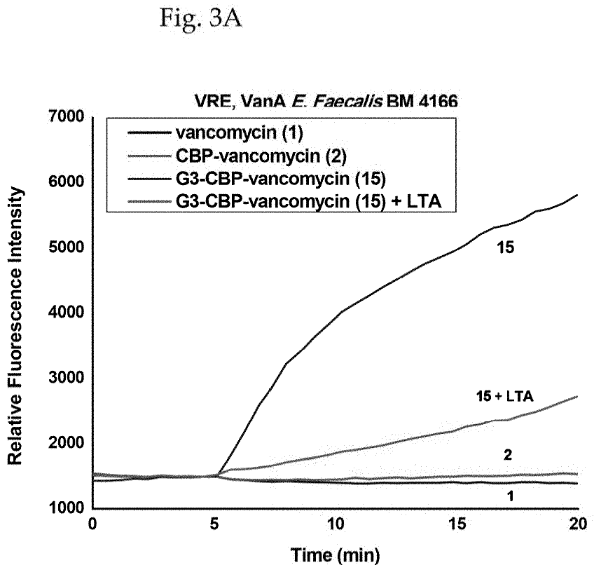

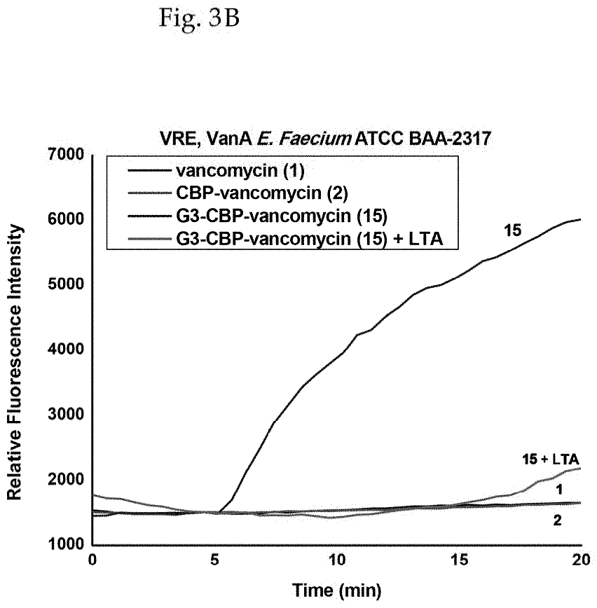

The presence of either NH₂CH₂CH₂CH₂NHC(═NH₂)NH₂⁺ (G3⁺, 100 μg/mL) or an inorganic divalent cation (Mg²⁺, 100 μg/mL) had no effect on these observations, revealing the requirement for covalent attachment of this G3 group to vancomycin for diverted binding. These results are shown in FIGS. 3A and 3B. In the case of vancomycin (not shown) and CBP-vancomycin and although not the objective of these studies, the addition of G3 also did not lead to an improvement in the activity, indicating that it must be incorporated into the molecule to have its impact (compare Compound 2+G3 vs Compound 15).

Unlike LTA, inorganic phosphate anion (H₂PO₄⁻, 100 μg/mL) or a phospholipid (POPE, 100 μg/mL) found in bacteria cell membranes did not alter the MIC of Compound 15, indicating that the diverted binding observed is selective for TA. Notably, exogeneous LTA only decreases the antimicrobial potency of G3-CBP-vancomycin (Compound 15) and GBn-CBP-vancomycin (Compound 19) to the level of CBP-vancomycin (Compound 2) rather than completely abolishing the activity.

These observations are in accordance with competitive binding to exogeneous LTA serving only to disrupt association with the target responsible for the guanidinium-derived mechanism of action. This was further confirmed in the bacteria cell permeabilization assay where the presence of exogenous LTA nearly or completely abolished the permeabilization induced by Compound 15 (FIGS. 3A and 3B).

Synergistic Activity of the Peripheral Modifications Requires Incorporation into Single Molecule.

With the establishment of this newly introduced mechanism of action by the guanidine modification, a study was conducted to determine whether the synergistic behavior of the two mechanisms of action attributable to the CBP and guanidine modifications requires the two to be located on the same glycopeptide molecule. The antimicrobial potency of a 1:1 mixture of CBP-vancomycin (Compound 2) and G3-vancomycin (Compound 5) or GBn-vancomycin (Compound 9) was tested alongside G3-CBP-vancomycin (Compound 15) and GBn-CBP-vancomycin (Compound 19) (Table 6, below).

TABLE 6
Antimicrobial activity of equimolar mixtures of CBP-vancomycin
(Compound 2) and C-terminus vancomycin analogues (Compounds
3, 5 and 9) against VanA E. faecalis and VanA E. faecium,
presented alongside the individual activity of Compounds
2, 3, 4, 5, 9, 15, 19; MIC (μg/mL)

| | | VanA |
|---|---|---|
| | VanA | E. faecium |
| | E. faecalis | ATCC |
| Compound | BM4166 | BAA-2317 |
| CBP-vancomycin (2) | 5 | 2.5 |
| G3-vancomycin (5) | 16 | 4 |
| G3-CBP-vancomycin (15) | 0.6 | 0.3 |
| CBP-vancomycin (2) + G3-vancomycin (5) | 5 + 5 | 2.5 + 2.5 |
| GBn-vancomycin (9) | 16 | 4 |
| GBn-CBP-vancomycin (19) | 0.6 | 0.3 |
| CBP-vancomycin (2) + GBn-vancomycin (9) | 5 + 5 | 2.5 + 2.5 |

Unlike the synergistic activity observed with Compounds 15 and 19, the equimolar mixtures of the singly modified vancomycins did not display this enhanced potency, exhibiting antimicrobial activity only at the level of the most potent compound in the mixture (CBP-vancomycin Compound 2). Thus, the expression of the synergistic activity observed with Compounds 15 and 19 requires that both peripheral modifications be incorporated in a single molecule. A study was also conducted with the earlier C1 trimethylammonium cation modification and provided analogous results (Table 6).

Although there was uncertainty about what to expect before conducting the study, the results with the singly modified antibiotic combinations are consistent with simple additive effects of the two antibiotics and observation of only the potency of the most active compound in the mixture. This typical behavior stands in contrast to the synergistic, and special, activity observed when both peripheral modifications are found in a single molecule

### Additional Key Properties.

Neither Compound 5 nor Compound 15 exhibited red blood cell hemolytic activity derived from cell membrane lysis or disruption even at concentrations >100-fold above their MICs. Similarly, neither Compound 5 nor Compound 15 displayed mammalian cell toxicity (IC₅₀>20 μM) when assessed for growth inhibition against the NIH/3T3 (mouse embryonic fibroblasts), HepG2 (human liver cancer cell line) and HCT116 (human colon cancer) cell lines.

Although a maximum tolerated dose (MTD) for Compound 15 was not established, the compound was found to be tolerated at doses up to and including 50 mg/kg in the following PK studies in mice. In earlier studies, a MTD was found of 75 mg/kg for CBP-vancomycin (Compound 2) and 50 mg/kg for C1-CBP-vancomycin (Compound 4). Not surprisingly given the safety and tolerability of oritavancin that bears a peripheral CBP substituent, the CBP-vancomycin (Compound 2) MTD in these studies was found to be only 4-fold lower than vancomycin (MTD=300 mg/kg).

The further addition of the trimethylammonium cation in Compound 4 did not significantly alter this tolerability and the addition of the guanidine modification in Compound 15 detailed herein proved to be at least as well tolerated if not better (MTD 50 mg/kg). Notably, CBP-vancomycin (Compound 2, about 100-fold), C1-CBP-vancomycin (Compound 4, about 1000-fold) and G3-CBP-vancomycin (Compound 15, about 1000-fold) are progressively more potent than vancomycin, and would be administered at accordingly much lower doses, making the small MTD distinctions even more impressive. These studies establish that there is no significant additional acute toxicity associated with the added guanidinium (or trimethylammonium) cation modification, a key question initial studies were designed to answer.

The in vivo PK properties of Compound 15 in mice (iv, n=3/time point @ 50 and 10 mg/kg) were established and conducted side-by-side with Compound 4 that was characterized earlier³⁸ (Table 7, below). These earlier studies revealed that Compound 4 (t_{1/2}=5 hours), bearing the CBP group and trimethylammonium cation, exhibited improved short terminal half-life (0.5-1.3 hour, t_{1/2}), lower exposure (AUC), consistent volume of distribution (V_{d}), and rapid clearance (CL) of vancomycin. Compound 4 also mitigated the poor dose proportionality⁴⁷ and extended terminal half-life (t_{1/2}) of CBP-vancomycin (Compound 2) that makes clinical administration of the structurally related drug oritavancin challenging.

Like vancomycin, Compounds 4 and 15 displayed well behaved dose proportional PK across all parameters, whereas CBP-vancomycin (Compound 2) does not, exhibiting a relatively lower plasma exposure at the higher dose. Compound 15 exhibited improved the short terminal half-life, lower exposure, and rapid clearance of vancomycin like Compound 4. Compound 15 displayed overall PK parameters similar to those of Compound 4, but was found to exhibit an improved Cₘₐₓ, now approaching that of vancomycin itself, and improved AUC, while maintaining the excellent terminal half-life in mice (t_{1/2} 4.3-4.4 hours).

Notably, the improved terminal half-life relative to vancomycin along with other properties illustrate that Compound 15 and its guanidine modification are not subject to rapid metabolism. These preliminary studies establish that Compound 15, bearing both the peripheral CBP group and guanidine modification, displays substantial in vivo PK improvements over both vancomycin (Compound 1) and CBP-vancomycin (Compound 2). These data are shown in Table 7, below.

TABLE 7
Comparison PK properties of Compounds 4 and 15.^{a}

| | C1-CBP-vancomycin (4) | G3-CBP-vancomycin (15) | |
|---|---|---|---|
| Parameter | 10 mg/kg | 10 mg/kg | 50 mg/kg |
| Cₘₐₓ (μg/mL) | 14.5 | 35.9 | 152 |
| tₘₐₓ (h) | 0.08 | 0.14 | 0.28 |
| AUC (μg-h/mL) | 48.4 | 66.0 | 312 |
| Vₛₛ (L/kg) | 0.64 | 0.41 | 0.35 |
| CL (L/h/kg) | 0.12 | 0.09 | 0.09 |
| MRT_{lNF}_{_{—}}_{obs} (h) | 5.6 | 3.9 | 4.6 |
| t_{1/2} (h) | 5.1 | 4.4 | 4.3 |

^{a}Compounds administered iv @ 50 or 10 mg/kg in mice (n = 3/time point, measured at 0.083, 0.25, 0.5, 1, 2, 4, 6, 8 and 24 hours).

### In Vivo Efficacy

An in vivo efficacy study of Compound 15 was conducted in neutropenic mice (n=5/dosed group) using a well-established thigh infection model with an especially challenging VanA VRSA strain (VRS2⁵⁰). This S. aureus strain is representative of an especially challenging class of antibiotic-resistant pathogens, being both multidrug-resistant (MRSA) and vancomycin-resistant (VRSA). Therefore, the results are remarkable especially with this challenging pathogen.

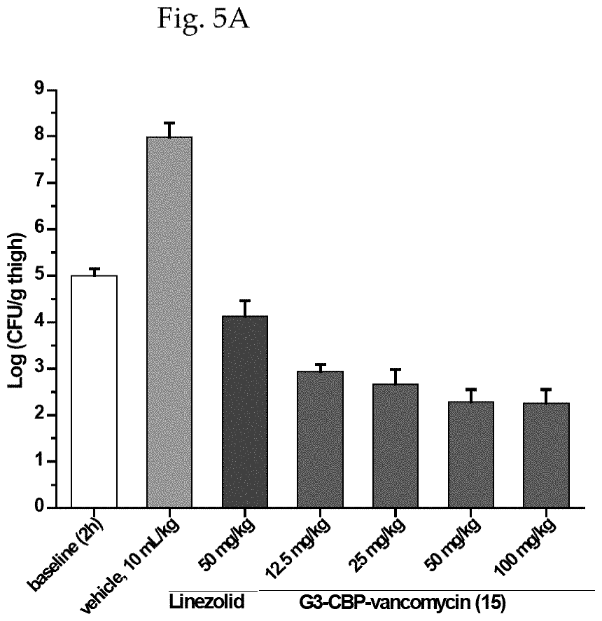

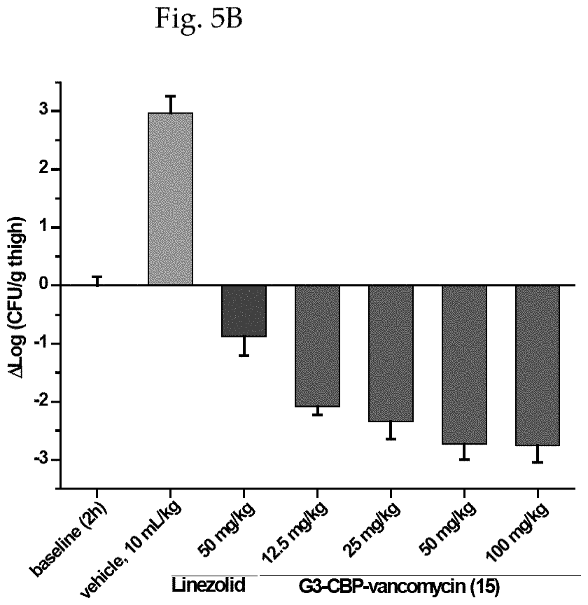

Following the experimental protocol of our previous in vivo efficacy study with C1-CBP-vancomycin (Compound 4)³⁸, the right thigh of each mouse was injected intramuscularly (im) with the pathogen. After 2 hours, control vehicle, linezolid (po, 50 mg/kg×2) and Compound 15 (sc, 12.5-100 mg/kg) were administered. After 26 hours, the bacterial CFU/g of harvested thighs was established and compared with baseline bacterial count at 2 hours and vehicle control at 26 hours.

Whereas vancomycin was found to be inactive in this model⁵⁰, a nearly 3-log₁₀ reduction in bacterial count was observed after treatment with Compound 15 (50 or 100 mg/kg), outperforming linezolid (administered po twice at 2 and 12 hours) (FIGS. 5A and 5B). Notably, a more than 2-log₁₀ reduction was observed even at the lower doses of Compound 15 (12.5 and 25 mg/kg), outperforming Compound 2 and Compound 4 at the same dose.³⁸

### CONCLUSIONS

Inspired by the discovery of the productive impact of a vancomycin C-terminus trimethylammonium cation modification and its underlying mechanism, an analogous and further improved guanidine modification was examined. The rationale for the studies, the evolution of the improvements, and the impact of this new peripheral modification on antimicrobial potency are summarized in Table 8, below.

TABLE 8
Evolution and comparison of the C-terminus cationic modifications examined.

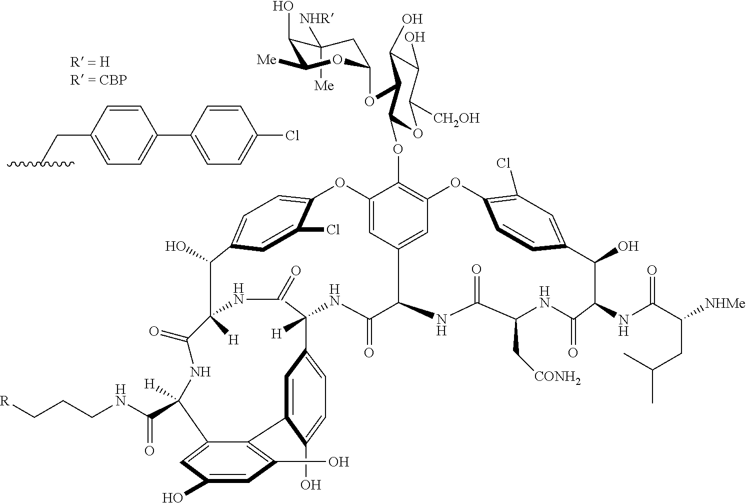

Antimicrobial Activity, MIC^{a} (μg/mL)

| VanA | |
|---|---|
| E. faecalis^{D} | E. faecium^{C} |

R′ = H

| | | |
|---|---|---|
| 1, vancomycin | 250 | 250 |
| 25, R = NMe₂ | 500 | 500 |
| 3, R = ⁺NMe₃ | 63 | 31 |
| 5, R = NHC(═NH₂)NH₂⁺ | 4 | 4 |

R′ = CBP

| | | |
|---|---|---|
| 2, CBP-vancomycin | 5 | 2.5 |
| 26, R = NMe₂ | 5 | 5 |
| 4, R = ⁺NMe₃ | 0.25 | 0.5 |
| 15, R = NHC(═NH₂)NH₂⁺ | 0.15 | 0.3 |

^{a}MIC = Minimum inhibitory conc.
^{D}BM 4166 or ATCC BAA-2573.
^{C}ATCC BAA-2317.

The C-terminus guanidine modification improves antimicrobial activity, enhances the durability of action against selection of resistance, and introduces a synergistic mechanism of action independent of D-Ala-D-Ala/D-Lac binding and inhibition of cell wall biosynthesis. The added mechanism of action results in induced bacterial cell permeability thought to involve interaction with teichoic acid in the cell envelope. For the first time, the synergistic behavior of the combined peripheral modifications utilized here are shown to require the presence of both the CBP and guanidinium modifications (or C1 modification) in a single molecule versus their combined use as an equimolar mixture of singly modified compounds.

A prototypical member of the series, G3-CBP-vancomycin (Compound 15), exhibits no hemolytic activity, displays no mammalian cell growth inhibition, possesses especially attractive in vivo PK properties, and exhibits exciting in vivo efficacy and potency against an especially challenging multidrug-resistant (MRSA) and VanA vancomycin-resistant (VRSA) S. aureus bacterial strain (VRS-2). Although the inspiration for the studies has been the incorporation of such combined peripheral modifications into pocket modified vancomycin analogues^{29-33,48-52}, their incorporation into vancomycin (Compound 1) or CBP-vancomycin (Compound 2) provide attractive new semisynthetic vancomycin analogues that act by two or three independent and synergistic mechanisms of action, only one of which is dependent on D-Ala-D-Ala binding.

Significantly, those compounds that contain the two combined peripheral modifications offer attractive opportunities for new treatments against not only vancomycin-sensitive, but especially for vancomycin-resistant bacteria even without incorporation of binding pocket modifications where they still can act by two synergistic and now durable mechanisms of action and display superb antimicrobial potencies (MIC 0.6-0.15 μg/mL, VanA VRE). As such, the C-terminus guanidine modifications found in G3-vancomycin (Compound 5), G3-CBP-vancomycin (Compound 15) and related analogues herein constitute new prototypical representatives of an exciting new class of vancomycin analogues that include not only earlier C1-vancomycin (Compound 3) and C1-CBP-vancomycin (Compound 4), but also other basic or cationic C-terminus modifications disclosed in the recent studies of Cooper⁵³ and Halder²⁴

### Materials and Methods

Reagents and solvents were purchased from commercial sources and used as received unless otherwise noted. ¹H and ¹³C NMR spectra were obtained using a Bruker Avance III HD 600 MHz spectrometer equipped with either a 5 mm QCI or 5 mm CPDCH probe or a Bruker Avance III 500 MHz spectrometer equipped with a 5 mm BBFO probe at 298° K. Residual solvent peaks were used as an internal reference. Coupling constants (J) (H, H) are given in Hz. Coupling patterns are designated as singlet (s), doublet (d), triplet (t), quadruplet (q), multiplet (m), or broad signal (br). High resolution mass spectra were measured with a TOF mass spectrometer. Analytical and preparative reverse-phase HPLC was performed using a Waters HPLC and an Agilent HPLC-MS. In vitro antimicrobial activity was determined on samples established to be 95% pure (HPLC: Nacalai Tesque, Inc., ARII-C18, 5 μm, 10×150 mm, 1-40% (analogues 5-14) or 20-80% (analogues 15-24) MeCN/H₂O-0.07% TFA gradient over 40 minutes, 3 mL/minute) through a standard microdilution assay. UV-Vis signal of hemolysis assay and cytotoxicity assay and flourescence signal of bacteria cell permeability assay was recorded with a TECAN plate reader.

Synthetic Procedures for Vancomycin Analogues Compounds 5-24, 27, 28

Scheme 1, below, shows a general synthetic route to preparation of the new C-terminus guanidine modified vancomycin analogues and also shows the structures of the new compounds.

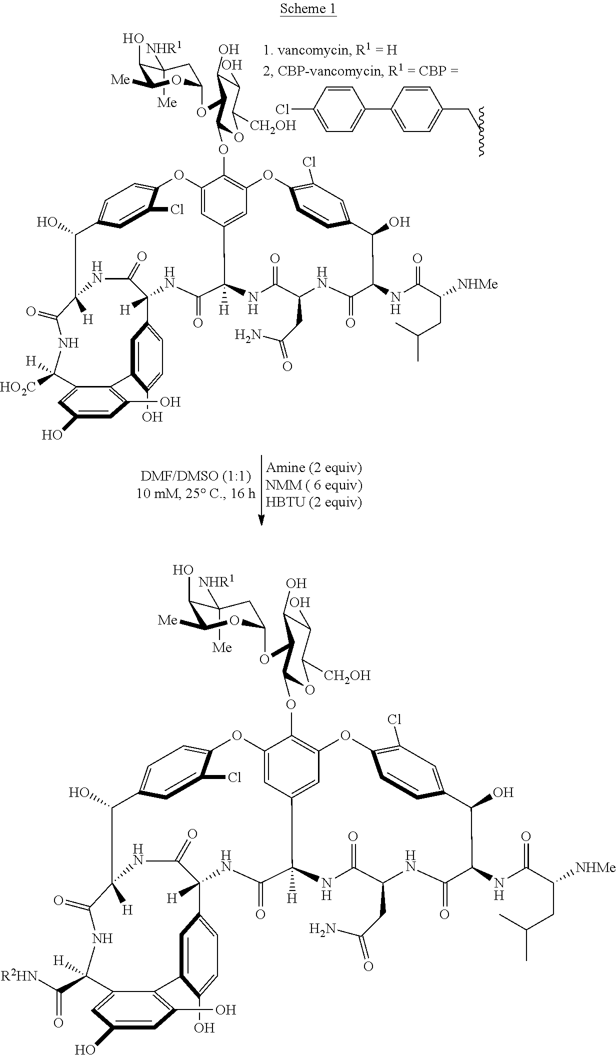

| Structure of R² | Abbreviation | Yield |
|---|---|---|
| 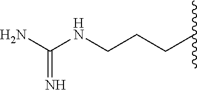 [SMILES: *CCCNC(=N)N] | G3 | R¹ = H, 5, 20% R¹ = CBP, 15, 61% |
| 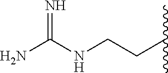 [SMILES: *CCNC(=N)N] | G2 | R¹ = H, 6, 57% R¹ = CBP, 16, 60% |
| 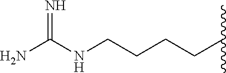 [SMILES: *CCCCNC(=N)N] | G4 | R¹ = H, 7, 76% R¹ = CBP, 17, 62% |
| 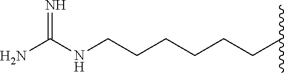 [SMILES: *CCCCCCNC(=N)N] | G6 | R¹ = H, 8, 69% R¹ = CBP, 18, 70% |
| 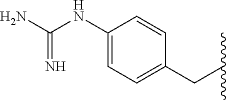 [SMILES: *Cc1ccc(NC(=N)N)cc1] | GBn | R¹ = H, 9, 54% R¹ = CBP, 19, 63% |
| 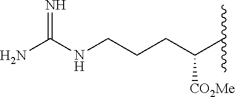 [SMILES: *[C@@H](CCCNC(=N)N)C(=O)OC] | Arg(OMe) | R¹ = H, 10, 75% R¹ = CBP, 20, 61% |
| 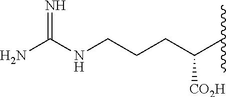 [SMILES: *[C@@H](CCCNC(=N)N)C(=O)O] | Arg^{a} | R¹ = H, 11, 70% R¹ = CBP, 21, 92% |
| 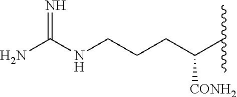 [SMILES: *[C@@H](CCCNC(=N)N)C(N)=O] | Arg(NH₂) | R¹ = H, 12, 31% R¹ = CBP, 22, 61% |
| 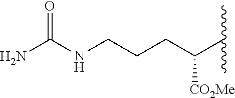 [SMILES: *[C@@H](CCCNC(N)=O)C(=O)OC] | Cit(OMe) | R¹ = H, 13, 52% R¹ = CBP, 23, 55% |
| 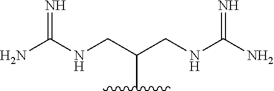 [SMILES: *C(CNC(=N)N)CNC(=N)N] | DiG | R¹ = H, 14, 12% R¹ = CBP, 24, 29% |

^{a}11 and 21 were obtained by LiOH-mediated hydrolysis of 10 and 20

Table 9, below, shows amino guanidine-terminated compounds used in the preparation of the C-terminal guanidine-containing CBP-vancomycin compounds that in part illustrate the compounds of the present invention, and their sources.

TABLE 9
Structure and source of guanidine-containing amines used

| | | Used in the synthesis | Source |
|---|---|---|---|
| Structure of amine | Abbreviation | of Compound | (Citation No.) |
| 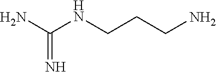 | G3 | 5, 15 | 54 |
| 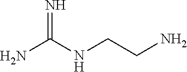 | G2 | 6, 16 | 54 |
| 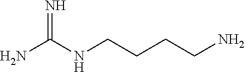 | G4 | 7, 17 | 55 |
| 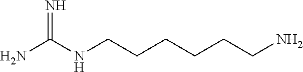 [SMILES: N=C(N)NCCCCCCN] | G6 | 8, 18 | 55 |
| 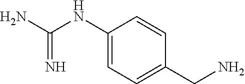 [SMILES: N=C(N)Nc1ccc(CN)cc1] | GBn | 9, 19 | 56 |
| 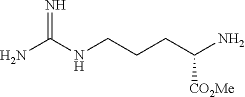 [SMILES: COC(=O)[C@@H](N)CCCNC(=N)N] | Arg(OMe) | 10, 20 | Commercial |
| 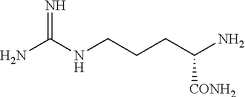 [SMILES: N=C(N)NCCC[C@H](N)C(N)=O] | Arg(NH₂) | 12, 22 | Commercial |
| 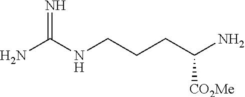 [SMILES: COC(=O)[C@@H](N)CCCNC(=N)N] | Cit(OMe) | 13, 23 | Commercial |
| 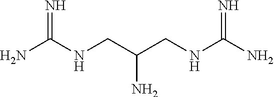 [SMILES: N=C(N)NCC(N)CNC(=N)N] | DiG | 14, 24 | Synthesis detailed |

### Synthesis of DiG

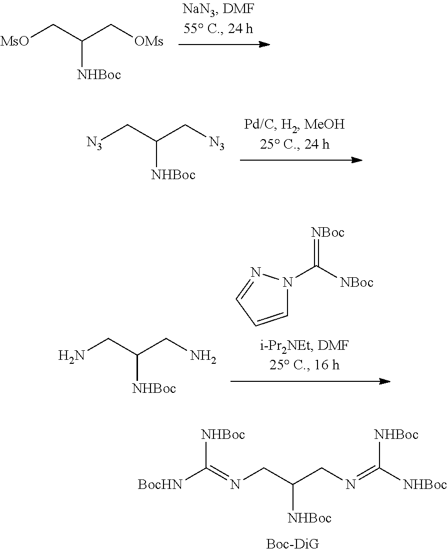

2-((tert-Butyloxycarbonyl)amino)propane-1,3-diyl dimethanesulfonate (2.08 g, 6 mmol) was dissolved in DMF (30 mL). NaN₃ (2.34 g, 36 mmol) was added, and the mixture was warmed at 55° C. for 24 hours before it was cooled to 25° C. and poured into H₂O (100 mL). The mixture was extracted with EtOAc (100 mL×2) and the organic layer dried over Na₂SO₄. The solvent was removed to yield crude Cert-butyl (1,3-diazidopropan-2-yl)carbamate as a colorless oil.

The crude product was dissolved in MeOH (60 mL) and 5% Pd/C (720 mg) was added. The mixture was stirred at 25° C. under 1 atm of H₂ for 24 hours before it was filtered over Celite®. The solvent was removed to yield crude Cert-butyl (1,3-diaminopropan-2-yl)carbamate as a colorless oil. This crude product was dissolved in DMF (30 mL) and N,N′-di-Boc-1H-pyrazole-1-carboxamidine (3.72 g, 12 mmol) and i-Pr₂NEt (1.55 g, 12 mmol) were added. The mixture was stirred at 25° C. for 16 hours. H₂O (100 mL) was added and the mixture was extracted with EtOAc (100 mL×2). The combined organic layer was dried over Na₂SO₄. The solvent was removed, and the residue was purified by column chromatography (SiO₂, EtOAc/hexanes=1:7) to provide Boc-DiG as a white solid (350 mg, 9% over 3 steps): ¹H NMR (600 MHz, CDCl₃) δ 11.45 (s, 2H), 8.58 (s, 2H), 6.16 (s, 1H), 3.94-3.87 (m, 1H), 3.70-3.62 (m, 2H), 3.52-3.44 (m, 2H), 1.49 (s, 18H), 1.48 (s, 18H), 1.43 (s, 9H); ¹³C NMR (600 MHz, CDCl₃) δ 163.4, 157.1, 156.3, 153.0, 83.4, 79.5, 51.8, 43.0, 30.0, 28.4, 28.2; ESI-TOF HRMS m/z 674.4091 ([M+H]⁺, [C₃₀H₅₅N₇O₁₀+H]⁺ requires 674.4089).

Boc-DiG (350 mg, 0.52 mmol) was dissolved in a mixture of CH₂Cl₂ and CF₃COOH (2 mL, V/V=1:1), and the mixture was stirred at 25° C. for 16 hours. The solvent was removed to give DiG (260 mg, 97%) as a colorless oil and the product was used without further purification.

### General Procedure for the Synthesis of 5-10 and 12-14

A solution of vancomycin hydrochloride (10.0 mg, 6.7 μmol) in DMF/DMSO (1/1, 670 μL) was treated sequentially with the corresponding amine (1 M in DMF/DMSO=1/1, 13.4 μL, 13.4 μmol, 2 equiv), N-methylmorpholine (distilled, 1 M in DMF/DMSO=1/1, 40.2 μL, 40.2 μmol, 6 equiv), and HBTU (0.5 M in DMF/DMSO=1/1, 26.8 μL, 13.4 μmol, 2 equiv). The mixture was stirred at 5° C. for 4 hours and quenched with the addition of H₂O (1 mL). The mixture was purified by semi-preparative reverse-phase HPLC (Nacalai Tesque, Inc., ARII-C18, 5 μm, 10×150 mm, 1-40% MeCN/H₂O-0.07% TFA gradient over 40 minutes, 3 mL/minute) to afford the desired products as a white solid.

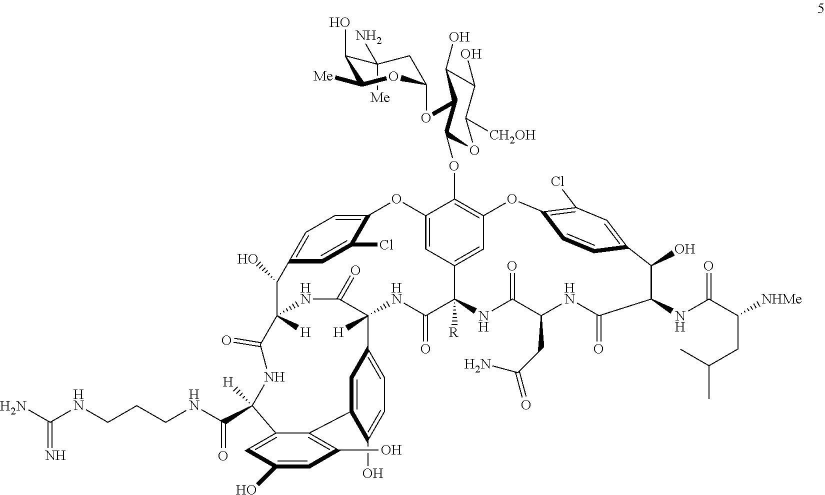

Compound 5 (2.5 mg, 20%, t_{R}=20.7 minutes; >95% HPLC purity): ¹H NMR (600 MHz, DMSO-d₆) δ 9.24 (s, 1H), 9.00 (s, 1H), 8.73-8.64 (m, 1H), 8.57-8.46 (m, 1H), 8.04 (s, 1H), 7.85 (m, 1H), 7.68 (s, 2H), 7.60 (t, J=6.0 Hz, 1H), 7.56 (d, J=8.4 Hz, 1H), 7.52-7.42 (m, 2H), 7.34 (d, J=8.4 Hz, 1H), 7.25 (d, J=11.3 Hz, 1H), 7.22-7.16 (m, 1H), 7.06 (s, br, 1H), 6.83-6.73 (m, 2H), 6.70 (d, J=8.5 Hz, 1H), 6.37 (d, J=2.2 Hz, 1H), 6.23 (d, J=2.2 Hz, 1H), 5.76 (d, J=7.6 Hz, 1H), 5.57 (d, J=17.0 Hz, 1H), 5.36-5.24 (m, 2H), 5.22 (m, 2H), 4.95 (s, 1H), 4.72-4.66 (m, 1H), 4.46 (d, J=5.3 Hz, 2H), 4.35 (d, J=5.3 Hz, 1H), 4.24 (d, J=11.3 Hz, 1H), 3.96 (s, 1H), 3.70 (dd, J=18.0, 8.7 Hz, 1H), 3.66-3.58 (m, 1H), 3.54 (q, J=8.7 Hz, 1H), 3.49-3.42 (m, 1H), 3.32-3.21 (m, 2H), 3.18-3.13 (m, 2H), 3.11 (q, J=6.2 Hz, 2H), 2.65 (s, 3H), 1.96-1.84 (m, 1H), 1.73 (d, J=13.5 Hz, 1H), 1.69-1.65 (m, 3H), 1.61-1.53 (m, 1H), 1.29 (d, J=14.0 Hz, 3H), 1.14-1.10 (m, 3H), 0.94-0.87 (m, 3H), 0.85 (dd, J=6.4, 2.3 Hz, 3H). ESI-TOF HRMS m/z 1546.5392 ([M+H]⁺, [C₇₀H₈₅Cl₃N₁₃O₂₃+H]⁺ requires 1546.5337).

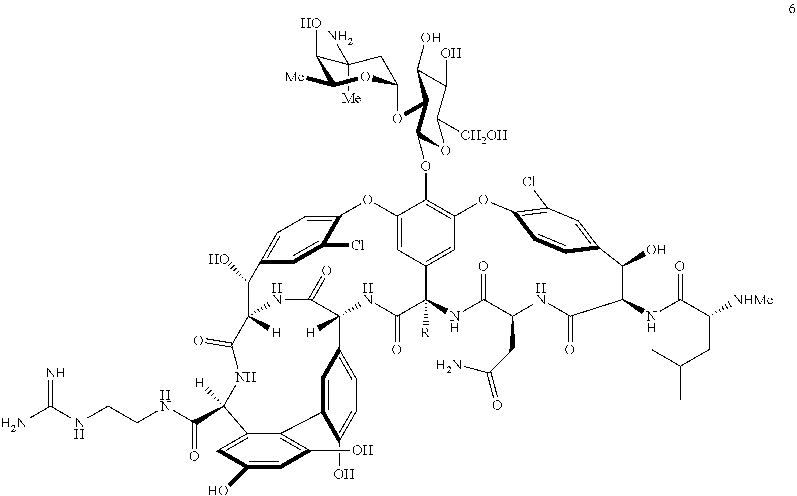

Compound 6 (7.3 mg, 57%, t_{R}=19.7 minutes; >95% HPLC purity): ¹H NMR (600 MHz, DMSO-d₆) δ 9.37 (s, 1H), 9.07 (s, 1H), 9.03 (s, 1H), 8.69 (s, 1H), 8.60-8.41 (m, 1H), 8.13 (s, 1H), 7.93-7.77 (m, 1H), 7.67 (s, 2H), 7.59 (t, J=5.7 Hz, 1H), 7.55 (d, J=8.0 Hz, 1H), 7.52-7.48 (m, 1H), 7.46 (dd, J=8.2, 1.8 Hz, 1H), 7.34 (d, J=8.3 Hz, 1H), 7.22 (s, 1H), 7.19 (d, J=8.3 Hz, 1H), 7.11-6.99 (m, 1H), 6.76 (dd, J=8.4, 2.0 Hz, 1H), 6.73 (s, 1H), 6.71 (d, J=8.4 Hz, 1H), 6.57 (s, br, 5H), 6.38 (d, J=2.3 Hz, 1H), 6.22 (d, J=2.2 Hz, 1H), 5.99 (s, 1H), 5.88 (d, J=5.9 Hz, 1H), 5.76 (d, J=7.9 Hz, 1H), 5.69-5.56 (m, 1H), 5.47 (s, 1H), 5.41-5.29 (m, 1H), 5.28-5.21 (m, 3H), 5.20-5.17 (m, 2H), 5.11 (s, 1H), 4.94 (s, 1H), 4.68 (q, J=7.0, 5.9 Hz, 1H), 4.45 (d, J=5.4 Hz, 1H), 4.38 (d, J=5.4 Hz, 1H), 4.23 (d, J=11.7 Hz, 2H), 4.05-3.91 (s, 1H), 3.74-3.61 (m, 1H), 3.55 (t, J=8.3 Hz, 1H), 3.28-3.25 (m, 2H), 3.25-3.20 (m, 2H), 3.18 (s, 1H), 2.64 (s, 3H), 2.23-2.07 (m, 1H), 1.90 (d, J=11.2 Hz, 1H), 1.83-1.71 (m, 1H), 1.71-1.66 (m, 1H), 1.66-1.60 (m, 1H), 1.56 (dt, J=12.6, 6.1 Hz, 1H), 1.30 (s, 3H), 1.07 (d, J=6.3 Hz, 3H), 0.91 (d, J=6.3 Hz, 3H), 0.86 (d, J=6.3 Hz, 3H). ESI-TOF HRMS m/z 1532.5173 ([M+H]⁺, [C₆₉H₈₃Cl₂N₁₃O₂₃+H]⁺ requires 1532.5174).

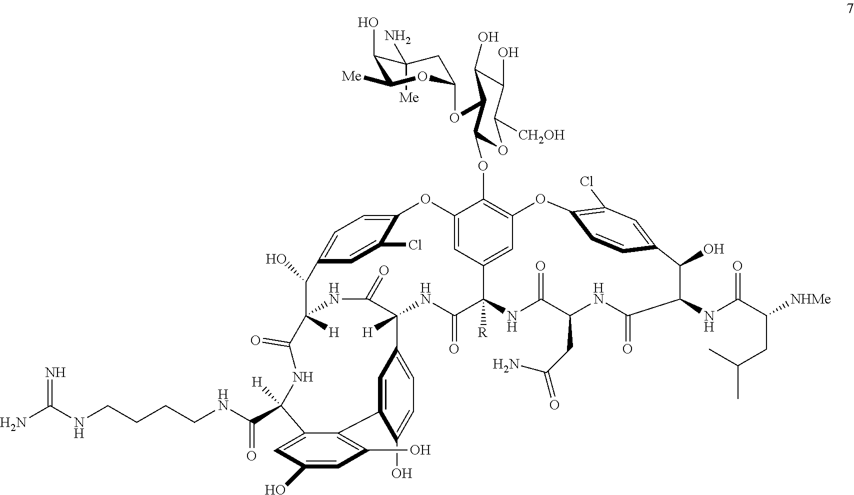

Compound 7 (9.8 mg, 76%, t_{R}=20.2 minutes; >95% HPLC purity): ¹H NMR (600 MHz, DMSO-d₆) δ 9.29 (s, 1H), 9.02 (s, 2H), 8.45 (s, 1H), 7.96 (s, 1H), 7.85 (s, 1H), 7.69 (s, 3H), 7.61-7.54 (m, 2H), 7.53-7.39 (m, 2H), 7.33 (d, J=8.4 Hz, 1H), 7.28-7.21 (m, 1H), 7.18 (d, J=8.4 Hz, 1H), 7.02 (s, br, 1H), 6.79-6.73 (m, 2H), 6.70 (d, J=8.4 Hz, 1H), 6.37 (d, J=2.2 Hz, 1H), 6.23 (d, J=2.2 Hz, 1H), 6.02 (s, 1H), 5.76 (d, J=7.9 Hz, 1H), 5.58 (s, 1H), 5.31-5.22 (m, 3H), 5.21-5.16 (m, 2H), 4.95 (s, 1H), 4.69 (dd, J=8.7, 4.5 Hz, 1H), 4.45 (d, J=5.5 Hz, 1H), 4.36 (d, J=5.5 Hz, 1H), 4.31-4.16 (m, 1H), 3.96 (s, 1H), 3.68 (d, J=10.8 Hz, 1H), 3.64 (s, 2H), 3.27 (d, J=4.9 Hz, 2H), 3.24-3.16 (m, 2H), 3.15-3.07 (m, 3H), 3.05 (s, 1H), 2.92 (s, 1H), 2.65 (s, 3H), 2.16 (d, J=11.3 Hz, 1H), 1.91 (d, J=10.2 Hz, 1H), 1.79-1.70 (m, 1H), 1.68 (q, J=7.0 Hz, 1H), 1.62 (q, J=6.3 Hz, 1H), 1.60-1.54 (m, 1H), 1.48 (s, 4H), 1.30 (s, 3H), 1.07 (d, J=6.2 Hz, 3H), 0.90 (d, J=6.2 Hz, 3H), 0.85 (dd, J=6.4, 2.5 Hz, 3H). ESI-TOF HRMS m/z 1560.5457 ([M+H]⁺, [C₇₁H₈₇Cl₂N₁₃O₂₃+H]⁺ requires 1560.5487).

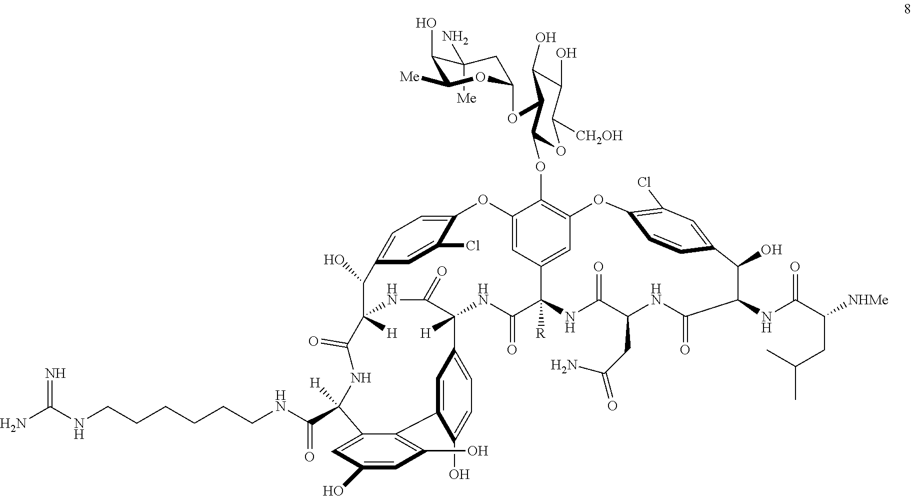

Compound 8 (9.0 mg, 69%, t_{R}=22.5 minutes; >95% HPLC purity): ¹H NMR (600 MHz, DMSO-d₆) δ 9.33 (s, 1H), 9.00 (s, 2H), 8.64 (s, 1H), 8.43 (s, 1H), 7.92-7.79 (m, 2H), 7.69 (t, J=5.5 Hz, 4H), 7.61-7.53 (m, 1H), 7.53-7.37 (m, 3H), 7.33 (d, J=8.2 Hz, 1H), 7.26-7.14 (m, 2H), 7.01 (s, 1H), 6.79-6.73 (m, 1H), 6.73-6.63 (m, 2H), 6.37 (d, J=2.2 Hz, 1H), 6.24 (d, J=2.2 Hz, 1H), 5.76 (d, J=7.8 Hz, 1H), 5.57 (s, 1H), 5.26 (s, 1H), 5.24 (d, J=8.4 Hz, 2H), 5.22-5.17 (m, 2H), 4.95 (s, 1H), 4.68 (d, J=6.0 Hz, 1H), 4.44 (s, 1H), 4.37 (d, J=5.4 Hz, 1H), 4.22 (d, J=11.8 Hz, 1H), 3.96 (s, 1H), 3.57-3.52 (m, 3H), 3.47-3.41 (m, 1H), 3.27 (d, J=4.7 Hz, 2H), 3.20 (d, J=5.2 Hz, 1H), 3.18-3.11 (m, 2H), 3.09 (q, J=6.6 Hz, 3H), 2.65 (s, 3H), 2.21-2.08 (m, 1H), 1.91 (d, J=10.8 Hz, 1H), 1.80-1.71 (m, 1H), 1.80-1.71 (m, 1H), 1.71-1.66 (m, 1H), 1.66-1.54 (m, 2H), 1.46 (s, 4H), 1.29 (d, J=7.7 Hz, 8H), 1.06 (d, J=6.3 Hz, 3H), 0.91-0.84 (m, 6H). ESI-TOF HRMS m/z 1588.5770 ([M+H]⁺, [C₇₃H₉₁Cl₂N₁₃O₂₃+H]⁺ requires 1588.5800).

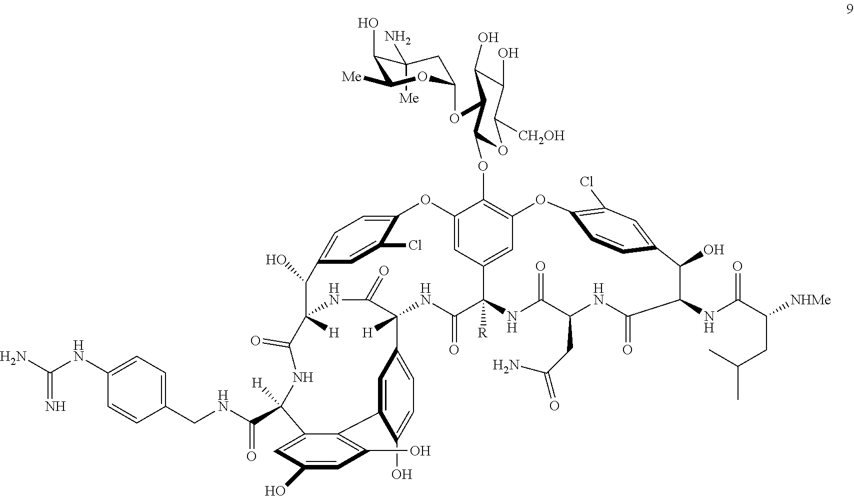

Compound 9 (7.0 mg, 54%, t_{R}=21.2 minutes; >95% HPLC purity): ¹H NMR (600 MHz, DMSO-d₆) δ 9.95 (s, 1H), 9.27 (s, 1H), 9.05 (s, 1H), 9.01 (s, 1H), 8.67 (s, 1H), 8.50 (s, 2H), 7.86 (s, 1H), 7.68 (s, 3H), 7.57 (d, J=8.1 Hz, 2H), 7.54 (s, 2H), 7.47 (d, J=8.5 Hz, 2H), 7.35 (d, J=8.1 Hz, 3H), 7.24 (s, 1H), 7.20 (d, J=8.4 Hz, 1H), 7.17 (d, J=8.3 Hz, 2H), 7.04 (s, 1H), 6.78 (d, J=7.9 Hz, 2H), 6.72 (d, J=8.7 Hz, 1H), 6.39 (d, J=2.2 Hz, 1H), 6.30 (d, J=2.1 Hz, 1H), 5.77 (d, J=7.8 Hz, 1H), 5.60 (s, 1H), 5.29-5.22 (m, 3H), 5.20 (d, J=6.4 Hz, 2H), 4.95 (s, 1H), 4.69 (q, J=6.5 Hz, 1H), 4.52-4.42 (m, 3H), 4.37-4.30 (m, 1H), 4.26 (d, J=12.3 Hz, 1H), 3.96 (s, 1H), 3.69 (d, J=10.8 Hz, 2H), 3.28 (d, J=4.2 Hz, 2H), 3.19 (s, 1H), 2.65 (s, 3H), 2.21-2.12 (m, 1H), 1.97-1.87 (m, 1H), 1.79-1.71 (m, 1H), 1.71-1.67 (m, 1H), 1.67-1.61 (m, 1H), 1.61-1.54 (m, 1H), 1.31 (s, 3H), 1.08 (d, J=6.2 Hz, 3H), 0.91 (d, J=6.2 Hz, 3H), 0.86 (d, J=6.2 Hz, 3H). ESI-TOF HRMS m/z 1594.5343 ([M+H]⁺, [C₇₄H₈₅Cl₂N₁₃O₂₃+H]⁺ requires 1594.5337).

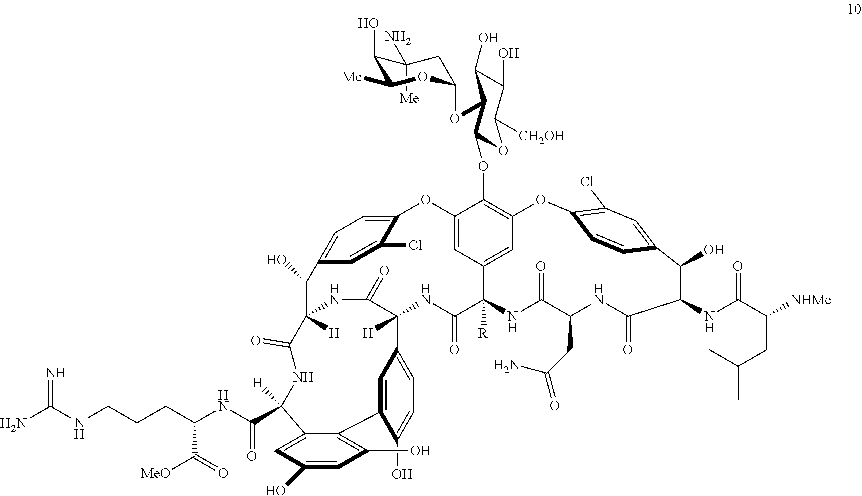

Compound 10 (9.9 mg, 75%, t_{R}=20.8 minutes; >95% HPLC purity): ¹H NMR (600 MHz, DMSO-d₆) δ 9.01 (s, 2H), 8.57 (s, 1H), 8.49 (d, J=7.5 Hz, 1H), 7.86 (d, J=5.0 Hz, 1H), 7.72 (s, 3H), 7.62 (t, J=5.5 Hz, 1H), 7.58 (s, 1H), 7.47 (t, J=8.7 Hz, 2H), 7.34 (d, J=8.2 Hz, 1H), 7.27-7.15 (m, 2H), 7.01 (s, 1H), 6.91-6.80 (m, 1H), 6.77 (d, J=8.4 Hz, 1H), 6.71 (dd, J=8.4, 3.4 Hz, 1H), 6.52-6.38 (m, 1H), 6.34 (s, 1H), 5.76 (d, J=7.8 Hz, 1H), 5.59-5.53 (m, 1H), 5.30-5.22 (m, 2H), 5.22-5.14 (m, 3H), 4.96 (s, 1H), 4.77-4.60 (m, 1H), 4.51 (d, J=5.6 Hz, 1H), 4.47 (s, 1H), 4.32 (q, J=7.6 Hz, 1H), 4.22 (d, J=11.8 Hz, 1H), 4.01-3.89 (m, 2H), 3.69 (s, 3H), 3.66-3.60 (m, 3H), 3.27 (s, 1H), 3.20 (s, 1H), 3.13-2.99 (m, 2H), 2.69 (s, 3H), 2.66 (s, 3H), 2.16 (s, 1H), 1.95-1.85 (m, 1H), 1.85-1.73 (m, 2H), 1.72-1.65 (m, 2H), 1.63-1.54 (m, 2H), 1.52-1.45 (m, 2H), 1.30 (s, 2H), 1.28 (s, 1H), 1.07 (d, J=6.2 Hz, 3H), 0.90 (d, J=5.0 Hz, 3H), 0.87-0.81 (m, 3H). ESI-TOF HRMS m/z 1618.5528 ([M+H]⁺, [C₇₃H₈₉Cl₂N₁₃O₂₅+H]⁺ requires 1618.5542).

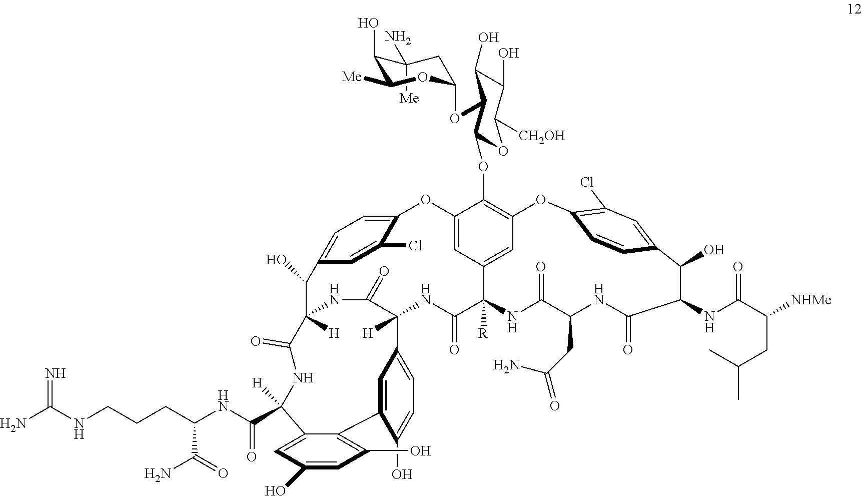

Compound 12 (4.0 mg, 31%, t_{R}=19.4 minutes; >95% HPLC purity): ¹H NMR (600 MHz, DMSO-d₆) δ 9.37 (s, 1H), 9.21 (s, br, 1H), 9.05 (s, 1H), 8.97 (s, 2H), 8.79-8.64 (m, 1H), 8.63-8.43 (m, 2H), 7.90 (d, J=8.0 Hz, 1H), 7.88-7.81 (m, 1H), 7.65 (s, br, 3H), 7.56 (d, J=7.3 Hz, 1H), 7.52 (t, J=5.8 Hz, 1H), 7.50-7.44 (m, 2H), 7.34 (dd, J=8.3, 2.5 Hz, 1H), 7.25 (s, 1H), 7.23 (d, J=5.7 Hz, 1H), 7.18 (s, 1H), 7.13-6.98 (m, 1H), 6.84 (d, J=11.3 Hz, 1H), 6.80-6.74 (m, 1H), 6.71 (d, J=8.4 Hz, 1H), 6.58 (s, 3H), 6.40 (d, J=2.3 Hz, 1H), 6.25 (d, J=2.2 Hz, 1H), 6.00 (s, 1H), 5.88 (d, J=5.6 Hz, 1H), 5.77 (d, J=7.8 Hz, 1H), 5.67-5.54 (m, 2H), 5.48 (s, 1H), 5.41-5.30 (m, 1H), 5.24 (dd, J=10.4, 5.8 Hz, 2H), 5.21-5.14 (m, 3H), 5.12 (s, 1H), 4.94 (s, 1H), 4.74-4.64 (m, 1H), 4.46 (d, J=5.4 Hz, 1H), 4.43 (d, J=5.5 Hz, 1H), 4.32 (q, J=7.6 Hz, 1H), 4.24 (d, J=9.6 Hz, 2H), 4.13-3.86 (m, 2H), 3.77-3.67 (m, 1H), 3.67-3.58 (m, 2H), 3.58-3.50 (m, 2H), 3.27 (d, J=5.0 Hz, 2H), 3.18 (s, 2H), 3.14-3.05 (m, 2H), 2.70-2.59 (m, 3H), 2.15 (s, 1H), 1.94-1.87 (m, 1H), 1.81-1.70 (m, 2H), 1.70-1.65 (m, 1H), 1.65-1.54 (m, 3H), 1.53-1.47 (m, 2H), 1.30 (s, 2H), 1.27 (s, 1H), 1.09-1.01 (m, 3H), 0.91 (dd, J=6.3, 1.8 Hz, 3H), 0.86 (dd, J=6.5, 2.5 Hz, 3H). ESI-TOF HRMS m/z 1603.5515 ([M+H]⁺, [C₇₂H₈₈Cl₂N₁₄O₂₄+H]⁺ requires 1603.5545).

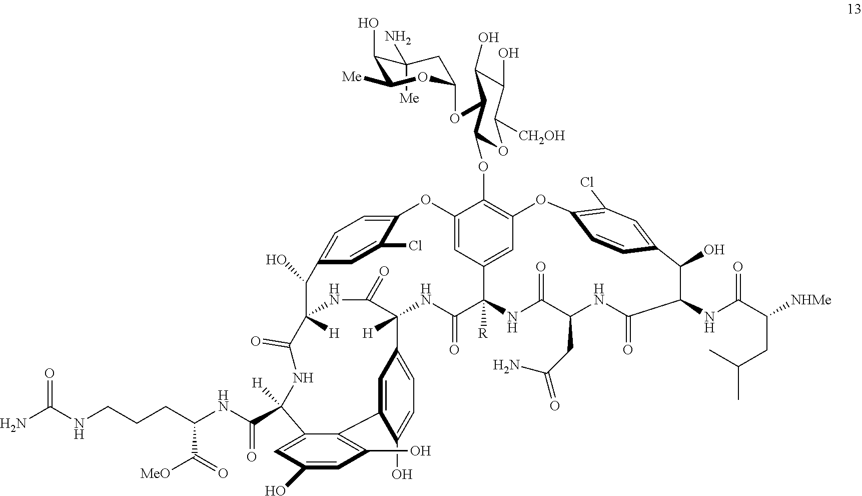

Compound 13 (6.4 mg, 52%, t_{R}=22.0 minutes; >95% HPLC purity): ¹H NMR (600 MHz, DMSO-d₆) δ 9.36 (s, 1H), 9.15 (s, 1H), 9.00 (s, 1H), 8.96 (s, 1H), 8.92 (s, 1H), 8.72 (s, 1H), 8.53 (s, 1H), 8.36 (d, J=7.4 Hz, 1H), 7.88 (d, J=3.3 Hz, 1H), 7.65 (s, 4H), 7.59-7.54 (m, 2H), 7.51 (s, 1H), 7.47 (t, J=8.4 Hz, 2H), 7.34 (d, J=8.7 Hz, 1H), 7.26-7.17 (m, 2H), 6.82-6.75 (m, 2H), 6.72 (t, J=8.2 Hz, 2H), 6.56 (s, 3H), 6.40 (d, J=2.3 Hz, 1H), 6.34 (d, J=2.2 Hz, 1H), 6.00 (s, 1H), 5.95 (s, 2H), 5.78 (d, J=7.9 Hz, 1H), 5.66-5.59 (m, 1H), 5.58 (s, 1H), 5.49 (s, 1H), 5.40 (s, 2H), 5.34 (d, J=7.2 Hz, 1H), 5.25 (t, J=6.3 Hz, 1H), 5.23-5.16 (m, 3H), 4.94 (s, 1H), 4.70 (d, J=7.1 Hz, 1H), 4.54 (d, J=5.9 Hz, 1H), 4.46 (d, J=5.4 Hz, 1H), 4.38-4.28 (m, 1H), 4.22 (d, J=12.2 Hz, 2H), 4.00-3.89 (m, 2H), 3.78-3.70 (m, 1H), 3.68 (s, 3H), 3.67-3.62 (m, 3H), 3.57-3.54 (m, 1H), 3.28 (s, 1H), 3.19 (s, 1H), 2.94 (q, J=9.5, 8.2 Hz, 3H), 2.65 (s, 3H), 2.19-2.11 (m, 1H), 1.93-1.90 (m, 1H), 1.79-1.73 (m, 2H), 1.73-1.67 (m, 2H), 1.67-1.61 (m, 2H), 1.60-1.56 (m, 1H), 1.45-1.34 1 (m, 2H), 1.30 (s, 1H), 1.28 (s, 2H), 1.11-1.05 (m, 3H), 0.92 (dd, J=6.5, 1.8 Hz, 3H), 0.87 (dd, J=6.5, 2.4 Hz, 3H). ESI-TOF HRMS m/z 1619.5377 ([M+H]⁺, [C₇₃H₈₈Cl₂N₁₂O₂₆+H]⁺ requires 1619.5382).

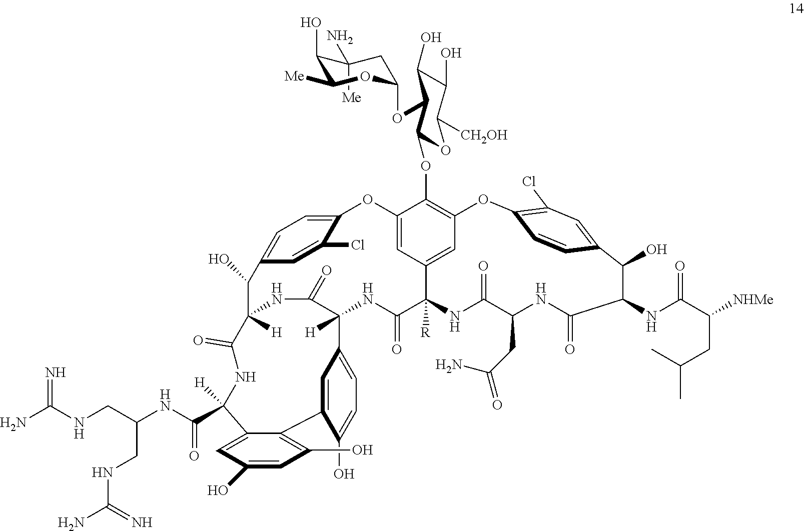

Compound 14 (1.7 mg, 12%, t_{R}=17.2 minutes; >95% HPLC purity): ¹H NMR (600 MHz, DMSO-d₆) δ 9.37 (s, 1H), 9.14 (s, 1H), 9.06 (s, 1H), 9.00 (s, 1H), 8.73 (s, 1H), 8.66 (s, 1H), 8.23 (d, J=8.0 Hz, 1H), 7.83 (s, 1H), 7.67 (s, 2H), 7.64 (s, 1H), 7.61-7.54 (m, 2H), 7.52 (d, J=9.2 Hz, 1H), 7.50-7.45 (m, 2H), 7.37 (dd, J=8.2, 3.7 Hz, 1H), 7.29 (s, 1H), 7.26 (d, J=8.2 Hz, 1H), 7.21 (d, J=8.4 Hz, 1H), 7.15-7.06 (m, 1H), 6.96-6.91 (m, 1H), 6.79 (d, J=8.2 Hz, 1H), 6.72 (d, J=8.5 Hz, 1H), 6.41 (d, J=2.2 Hz, 1H), 6.32 (s, 1H), 6.06 (s, 1H), 5.98 (s, 1H), 5.78 (d, J=8.0 Hz, 1H), 5.68-5.56 (m, 1H), 5.49 (dd, J=14.8, 6.7 Hz, 1H), 5.36 (dd, J=11.8, 6.5 Hz, 1H), 5.28 (s, 1H), 5.25 (s, 1H), 5.20 (s, 2H), 5.13 (s, 1H), 4.93 (s, 1H), 4.69 (t, J=6.6 Hz, 1H), 4.49 (s, 1H), 4.37 (d, J=4.9 Hz, 1H), 4.31 (d, J=11.8 Hz, 1H), 4.15-4.03 (m, 1H), 4.00-3.90 (m, 1H), 3.76-3.67 (m, 1H), 3.67-3.63 (m, 1H), 3.58-3.51 (m, 2H), 3.21-3.18 (m, 1H), 3.18 (d, J=3.2 Hz, 1H), 2.64 (s, 3H), 2.21-2.09 (m, 1H), 1.98-1.88 (m, 1H), 1.80-1.72 (m, 1H), 1.72-1.60 (m, 2H), 1.60-1.53 (m, 1H), 1.31 (s, 2H), 1.29 (s, 1H), 1.08 (d, J=6.3 Hz, 3H), 0.92 (d, J=6.0 Hz, 3H), 0.89-0.86 (d, J=6.0 Hz, 3H). ESI-TOF HRMS m/z 1603.5669 ([M+H]⁺, [C₇₁H₈₈Cl₂N₁₆O₂₃+H]⁺ requires 1603.5658).

### General Procedure for the Synthesis of 15-20 and 22-24

A solution of CBP-vancomycin.2CF₃COOH (2, 12.6 mg, 6.7 μmol) in DMF/DMSO (1/1, 670 μL) was treated sequentially with the corresponding amine (1 M in DMF/DMSO=1/1, 13.4 μL, 13.4 μmol, 2 equiv), N-methylmorpholine (distilled, 1 M in DMF/DMSO=1/1, 40.2 μL, 40.2 μmol, 6 equiv), and HBTU (0.5 M in DMF/DMSO=1/1, 26.8 μL, 13.4 μmol, 2 equiv). The mixture was stirred at 5° C. for 4 hours and quenched with the addition of H₂O (1 mL). The mixture was purified by semi-preparative reverse-phase HPLC (Nacalai Tesque, Inc., ARII-C18, 5 μm, 10×150 mm, 20-80% MeCN/H₂O-0.07% TFA gradient over 40 min, 3 mL/minute) to afford the desired products as a white solid.

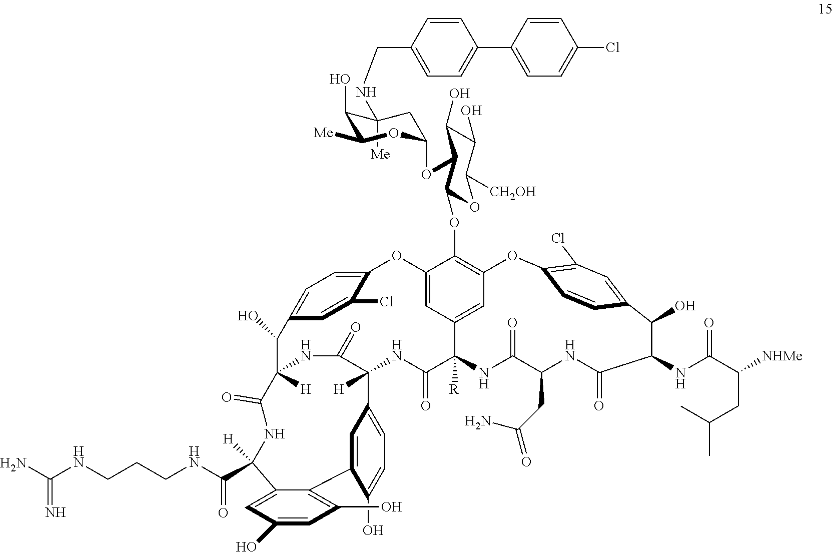

Compound 15 (8.5 mg, 61%, t_{R}=13.1 minutes; >95% HPLC purity): ¹H NMR (600 MHz, DMSO-d₆) δ 9.33 (m, 1H), 9.07 (s, 1H), 9.03 (s, 1H), 8.72-8.65 (m, 1H), 8.52 (s, 1H), 8.23 (s, 1H), 8.05 (s, 1H), 7.89-7.83 (m, 1H), 7.73 (m, 4H), 7.64 (t, J=6.0 Hz, 1H), 7.60-7.57 (m, 3H), 7.55-7.52 (m, 2H), 7.48 (t, J=8.0 Hz, 2H), 7.34 (dd, J=8.3, 3.2 Hz, 1H), 7.29-7.17 (m, 2H), 6.78 (m, 2H), 6.71 (d, J=8.4 Hz, 1H), 6.38 (s, 1H), 6.24 (s, 1H), 5.94-5.82 (m, 1H), 5.77 (d, J=7.9 Hz, 1H), 5.65-5.56 (m, 1H), 5.49-5.35 (m, 1H), 5.29 (d, J=10.8 Hz, 2H), 5.25-5.16 (m, 2H), 4.96 (s, 1H), 4.77-4.63 (m, 1H), 4.56-4.42 (m, 1H), 4.35 (d, J=5.1 Hz, 1H), 4.31-4.21 (m, 1H), 4.10-3.91 (m, 1H), 3.77-3.63 (m, 2H), 3.49 (s, 2H), 3.34-3.21 (m, 2H), 3.19-3.08 (m, 3H), 2.65 (s, 3H), 2.15 (s, 2H), 1.88-1.84 (m, 1H), 1.71-1.62 (m, 3H), 1.58 (d, J=7.5 Hz, 1H), 1.52-1.48 (m, 3H), 1.14 (d, J=6.2 Hz, 3H), 0.91 (d, J=6.2 Hz, 3H), 0.86 (dd, J=6.5, 2.6 Hz, 3H). ESI-TOF HRMS m/z 1746.5767 ([M+H]⁺, [C₈₃H₉₄Cl₃N₁₃O₂₃+H]⁺ requires 1746.5729).

### Scaled Synthesis of Compound 15

A solution of CBP-vancomycin.2CF₃COOH (315 mg, 0.166 mmol) in DMF/DMSO (1/1, 17 mL) was treated sequentially with H₂NCH₂CH₂CH₂NHC(NH₂)═NH₂+(G3⁺, 1 M in DMF/DMSO=1/1, 0.33 mL, 0.33 mmol, 2 equiv), N-methylmorpholine (distilled, 1 M in DMF/DMSO=1/1, 1.0 mL, 1.0 mmol, 6 equiv), and HBTU (0.5 M in DMF/DMSO=1/1, 0.67 mL, 0.33 mmol, 2 equiv). The mixture was stirred at 25° C. for 16 hours and quenched with the addition of H₂O (2 mL). The mixture was purified by semi-preparative reverse-phase HPLC (Vydac 218TP1022-C18, 10 μm, 22×250 mm, 20-80% MeCN/H₂O-0.07% TFA gradient over 40 min, 10 mL/min, t_{R}=14.6 minutes) to afford Compound 15 (195 mg) as a white solid.

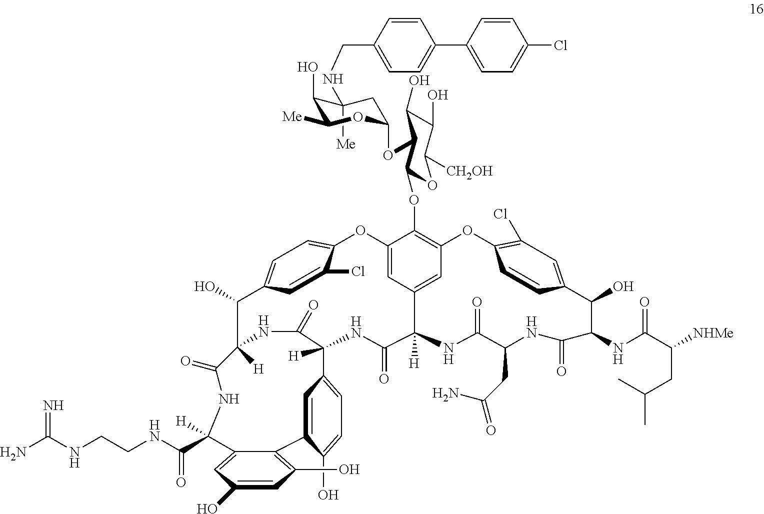

Compound 16 (8.4 mg, 60%, t_{R}=12.8 minutes; >95% HPLC purity): ¹H NMR (600 MHz, DMSO-d₆) δ 9.37 (s, 1H), 9.17 (s, 1H), 9.08 (s, 1H), 9.04 (s, 1H), 9.00 (s, 1H), 8.76 (s, 1H), 8.70 (s, 1H), 8.52 (s, 1H), 8.22 (s, 1H), 8.13 (t, J=5.9 Hz, 1H), 7.87 (d, J=2.0 Hz, 1H), 7.78-7.70 (m, 4H), 7.58 (d, J=8.0 Hz, 4H), 7.54 (d, J=8.4 Hz, 2H), 7.48 (d, J=8.4 Hz, 1H), 7.34 (dd, J=8.3, 3.9 Hz, 1H), 7.22 (d, J=8.0 Hz, 2H), 7.07 (s, 1H), 6.80-6.73 (m, 2H), 6.71 (d, J=8.5 Hz, 1H), 6.58 (s, 2H), 6.38 (d, J=2.2 Hz, 1H), 6.23 (d, J=2.1 Hz, 1H), 6.01 (d, J=12.2 Hz, 1H), 5.91 (d, J=6.0 Hz, 1H), 5.83 (s, 1H), 5.77 (d, J=7.9 Hz, 1H), 5.63 (s, 1H), 5.37 (d, J=7.7 Hz, 1H), 5.30 (s, 1H), 5.25 (s, 1H), 5.22-5.16 (m, 2H), 4.95 (s, 1H), 4.69 (q, J=6.5 Hz, 1H), 4.47 (s, 1H), 4.38 (d, J=5.3 Hz, 1H), 4.25 (d, J=12.1 Hz, 2H), 4.13 (s, 1H), 4.09-4.01 (m, 2H), 3.96 (s, 1H), 3.79-3.62 (m, 2H), 3.59 (t, J=8.5 Hz, 1H), 3.54 (s, 1H), 3.48 (s, 2H), 3.31-3.27 (m, 4H), 2.64 (s, 3H), 2.54 (s, 1H), 2.21-2.09 (m, 2H), 1.91-1.79 (m, 1H), 1.73-1.66 (m, 1H), 1.66-1.60 (m, 1H), 1.60-1.54 (m, 1H), 1.52 (s, 2H), 1.48 (s, 1H), 1.14 (d, J=6.2 Hz, 3H), 0.91 (d, J=6.2 Hz, 3H), 0.86 (t, J=4.4 Hz, 3H). ESI-TOF HRMS m/z 1732.5573 ([M+H]⁺, [C₈₂H₉₂Cl₃N₁₃O₂₃+H]⁺ requires 1732.5567).

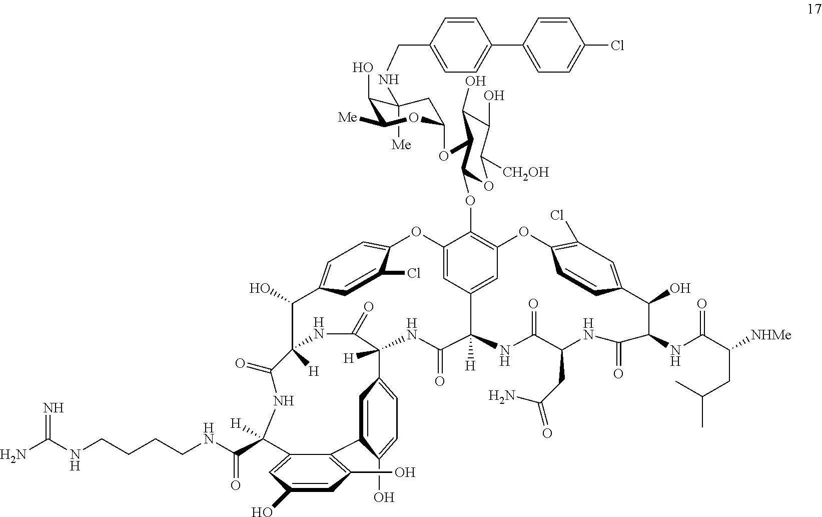

Compound 17 (8.8 mg, 62%, t_{R}=13.1 minutes; >95% HPLC purity): ¹H NMR (600 MHz, DMSO-d₆) δ 9.35 (s, 1H), 9.20 (s, 1H), 9.03 (s, 1H), 9.00 (s, 1H), 8.77 (s, 1H), 8.73-8.65 (m, 1H), 8.47 (s, 1H), 8.22 (s, 1H), 7.97-7.93 (m, 1H), 7.87 (s, 1H), 7.73 (dd, J=11.4, 8.4 Hz, 4H), 7.63-7.56 (m, 3H), 7.54 (d, J=8.5 Hz, 3H), 7.49 (d, J=8.3 Hz, 2H), 7.33 (dd, J=8.3, 3.8 Hz, 1H), 7.22 (d, J=8.7 Hz, 2H), 7.05 (s, br, 1H), 6.81-6.73 (m, 2H), 6.71 (d, J=8.5 Hz, 1H), 6.37 (d, J=2.3 Hz, 1H), 6.24 (d, J=2.1 Hz, 1H), 6.09-5.89 (m, 1H), 5.77 (d, J=7.9 Hz, 1H), 5.61 (s, 1H), 5.37 (d, J=7.3 Hz, 1H), 5.33-5.28 (m, 1H), 5.26 (s, 1H), 5.19 (dt, J=7.8, 4.7 Hz, 2H), 5.02-4.85 (m, 1H), 4.69 (q, J=6.5 Hz, 1H), 4.47 (s, 1H), 4.37 (d, J=5.4 Hz, 1H), 4.24 (d, J=11.9 Hz, 2H), 4.10-4.00 (m, 2H), 3.96 (s, 1H), 3.74-3.64 (m, 2H), 3.59 (t, J=8.5 Hz, 2H), 3.31-3.26 (m, 2H), 3.20 (dd, J=13.3, 6.6 Hz, 1H), 3.15-3.07 (m, 3H), 2.93 (s, 1H), 2.65 (s, 3H), 2.20-2.08 (m, 2H), 1.93-1.79 (m, 1H), 1.74-1.66 (m, 1H), 1.66-1.60 (m, 1H), 1.60-1.54 (m, 1H), 1.52 (s, 3H), 1.50-1.43 (m, 4H), 1.14 (d, J=6.2 Hz, 3H), 0.91 (d, J=6.2 Hz, 3H), 0.89-0.82 (m, 3H). ESI-TOF HRMS m/z 1760.5835 ([M+H]⁺, [C₈₄H₉₆Cl₃N₁₃O₂₃+H]⁺ requires 1760.5880).

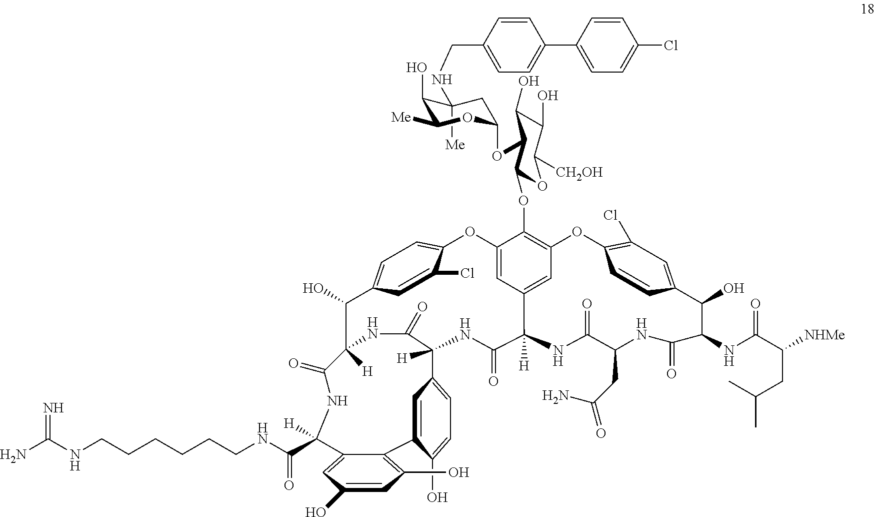

Compound 18 (10.0 mg, 70%, t_{R}=13.5 minutes; >95% HPLC purity): ¹H NMR (600 MHz, DMSO-d₆) δ 9.34 (s, 1H), 9.27 (s, 1H), 9.01 (s, 3H), 8.79 (s, 1H), 8.72-8.60 (m, 1H), 8.45 (s, 1H), 8.22 (s, 1H), 7.93-7.83 (m, 2H), 7.76-7.69 (m, 4H), 7.63 (t, J=5.7 Hz, 1H), 7.58 (d, J=7.8 Hz, 3H), 7.54 (d, J=8.4 Hz, 3H), 7.48 (t, J=7.7 Hz, 2H), 7.33 (dd, J=8.4, 3.6 Hz, 1H), 7.29-7.22 (m, 1H), 7.21 (s, 1H), 7.05 (s, br, 1H), 6.80-6.73 (m, 1H), 6.70 (d, J=8.9 Hz, 2H), 6.58 (s, br, 3H), 6.37 (d, J=2.3 Hz, 1H), 6.24 (d, J=2.2 Hz, 1H), 6.02 (s, 1H), 5.92 (s, 1H), 5.84 (s, 1H), 5.77 (d, J=7.8 Hz, 1H), 5.67-5.56 (m, 1H), 5.46-5.35 (m, 1H), 5.30 (t, J=6.3 Hz, 1H), 5.26 (s, 1H), 5.23-5.08 (m, 2H), 4.95 (s, 1H), 4.76-4.64 (m, 1H), 4.46 (d, J=5.1 Hz, 1H), 4.37 (d, J=5.5 Hz, 1H), 4.23 (d, J=12.0 Hz, 2H), 4.06 (s, 2H), 4.00-3.87 (m, 1H), 3.78-3.63 (m, 3H), 3.59 (t, J=8.5 Hz, 2H), 3.54 (s, 2H), 3.31-3.26 (m, 2H), 3.17 (s, 2H), 3.13-3.06 (m, 3H), 2.65 (s, 3H), 2.14 (s, 2H), 1.91-1.79 (m, 1H), 1.73-1.66 (m, 1H), 1.63 (t, J=6.5 Hz, 1H), 1.57 (dd, J=10.3, 4.5 Hz, 1H), 1.52 (s, 2H), 1.48 (s, 2H), 1.46 (d, J=7.5 Hz, 3H), 1.32-1.25 (m, 4H), 1.16-1.09 (m, 3H), 0.91 (d, J=6.1 Hz, 3H), 0.86 (dd, J=6.4, 2.6 Hz, 3H). ESI-TOF HRMS m/z 1788.6130 ([M+H]⁺, [C₈₆H₁₀₀Cl₃N₁₃O₂₃+H]⁺ requires 1788.6193).

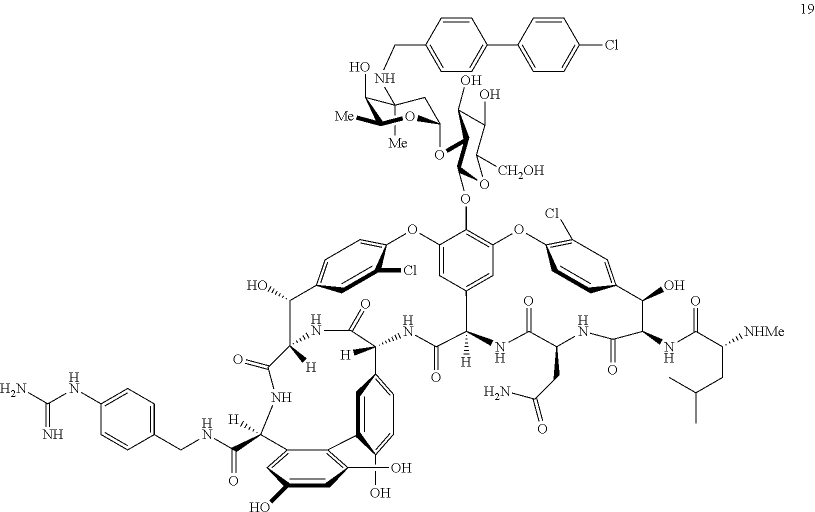

Compound 19 (9.0 mg, 63%, t_{R}=13.4 minutes; >95% HPLC purity): ¹H NMR (600 MHz, DMSO-d₆) δ 9.93 (s, 1H), 9.23 (s, 1H), 9.03 (s, 2H), 8.78 (s, 1H), 8.70 (s, 1H), 8.53-8.47 (m, 2H), 8.23 (s, 1H), 7.92-7.84 (m, 1H), 7.78-7.71 (m, 4H), 7.59 (d, J=7.8 Hz, 3H), 7.56-7.54 (m, 3H), 7.52 (s, 3H), 7.49 (d, J=9.0 Hz, 2H), 7.35 (d, J=8.4 Hz, 3H), 7.30-7.22 (m, 2H), 7.17 (d, J=8.4 Hz, 2H), 7.05 (s, 1H), 6.87-6.76 (m, 2H), 6.72 (d, J=8.5 Hz, 1H), 6.39 (d, J=2.2 Hz, 1H), 6.31 (d, J=2.2 Hz, 1H), 5.79 (d, J=7.8 Hz, 1H), 5.64 (s, 1H), 5.38 (d, J=7.5 Hz, 1H), 5.33-5.28 (m, 1H), 5.27 (s, 1H), 5.25-5.16 (m, 2H), 4.96 (s, 1H), 4.74-4.66 (m, 1H), 4.53-4.44 (m, 3H), 4.39-4.30 (m, 1H), 4.28 (d, J=12.2 Hz, 1H), 4.10-4.01 (m, 2H), 3.97 (s, 1H), 3.50 (s, 2H), 3.34-3.25 (m, 2H), 2.65 (s, 3H), 2.20-2.11 (m, 2H), 1.91-1.81 (m, 1H), 1.74-1.67 (m, 1H), 1.67-1.61 (m, 1H), 1.61-1.55 (m, 1H), 1.52 (s, 2H), 1.49 (s, 1H), 1.20-1.10 (m, 3H), 0.92 (d, J=6.2 Hz, 3H), 0.87 (dd, J=6.4, 2.4 Hz, 3H). ESI-TOF HRMS m/z 1794.5762 ([M+H]⁺, [C₈₇H₉₄Cl₃N₁₃O₂₃+H]⁺ requires 1794.5729).

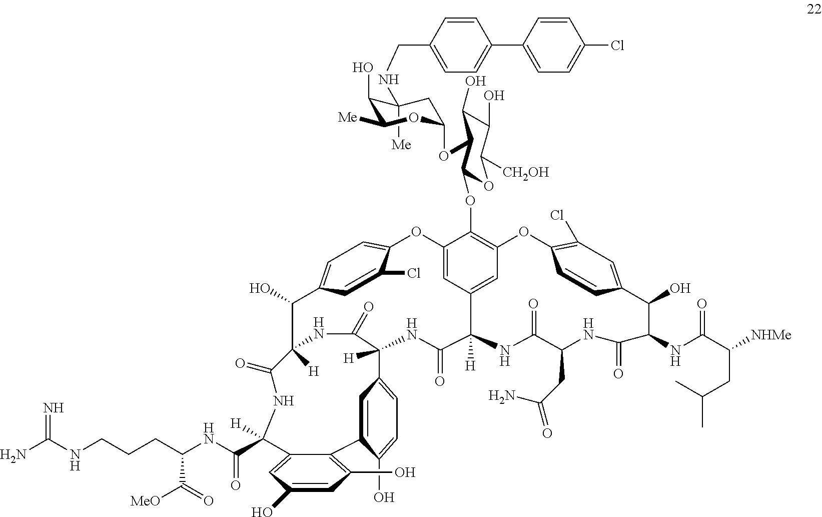

Compound 20 (8.9 mg, 61%, t_{R}=12.7 minutes; >95% HPLC purity): ¹H NMR (600 MHz, DMSO-d₆) δ 9.33 (s, 2H), 9.02 (s, 2H), 8.98 (s, 1H), 8.83 (s, 1H), 8.68 (s, 1H), 8.59 (s, 1H), 8.48 (d, J=7.4 Hz, 1H), 8.23 (s, 1H), 7.88 (s, 1H), 7.73 (d, J=6.1 Hz, 2H), 7.72 (d, J=5.8 Hz, 2H), 7.61-7.56 (m, 4H), 7.54 (d, J=8.4 Hz, 2H), 7.49 (t, J=8.1 Hz, 2H), 7.35 (d, J=2.6 Hz, 1H), 7.30-7.20 (m, 2H), 6.84 (d, J=10.8 Hz, 1H), 6.81-6.75 (m, 1H), 6.72 (d, J=8.5 Hz, 1H), 6.63 (s, 2H), 6.41 (d, J=2.2 Hz, 1H), 6.34 (d, J=2.1 Hz, 1H), 6.06 (s, 1H), 5.96 (s, 1H), 5.88 (s, 1H), 5.78 (d, J=7.9 Hz, 1H), 5.72-5.55 (m, 1H), 5.41 (d, J=7.1 Hz, 1H), 5.29 (s, 1H), 5.22-5.15 (m, 3H), 4.96 (s, 1H), 4.70 (q, J=6.2 Hz, 1H), 4.52 (d, J=5.6 Hz, 1H), 4.49 (s, 1H), 4.32 (q, J=7.6 Hz, 1H), 4.24 (d, J=12.2 Hz, 1H), 4.10-4.00 (m, 2H), 4.00-3.92 (m, 2H), 3.69 (s, 3H), 3.67 (s, 2H), 3.49 (s, 2H), 3.29 (s, 1H), 3.12-3.01 (m, 2H), 2.65 (s, 3H), 2.18-2.11 (m, 2H), 1.91-1.82 (m, 1H), 1.82-1.74 (m, 1H), 1.72-1.65 (m, 2H), 1.65-1.61 (m, 1H), 1.61-1.55 (m, 1H), 1.51 (s, 2H), 1.49 (s, 3H), 1.14 (d, J=6.1 Hz, 3H), 0.91 (d, J=6.0 Hz, 3H), 0.86 (d, J=6.1 Hz, 3H). ESI-TOF HRMS m/z 1818.5902 ([M+H]⁺, [C₈₆H₉₈Cl₃N₁₃O₂₅+H]⁺ requires 1818.5935).

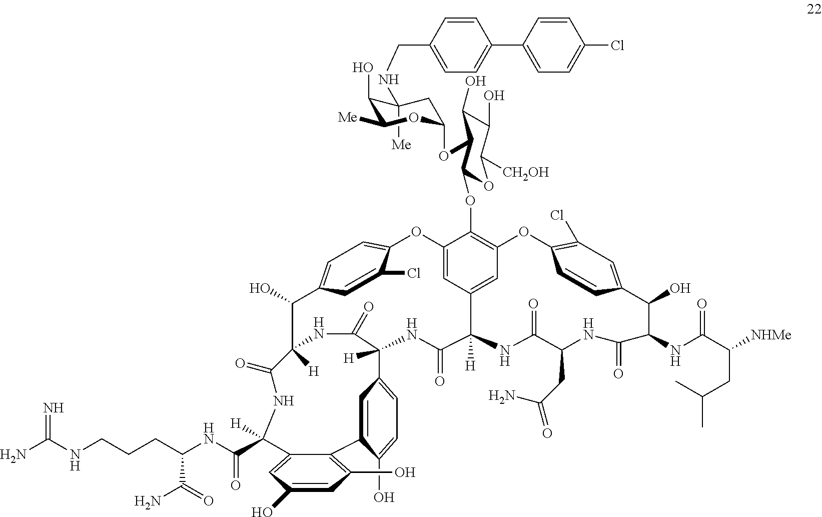

Compound 22 (8.8 mg, 61%, t_{R}=12.5 minutes; >95% HPLC purity): ¹H NMR (600 MHz, DMSO-d₆) δ 9.37 (s, 1H), 9.20 (s, 1H), 9.05 (s, 1H), 8.98 (s, 1H), 8.87-8.67 (m, 2H), 8.59 (s, 1H), 8.21 (s, 1H), 7.99-7.83 (m, 2H), 7.77-7.69 (m, 4H), 7.62-7.56 (m, 3H), 7.53 (d, J=7.9, 3H), 7.49 (t, J=8.1 Hz, 2H), 7.40-7.31 (m, 2H), 7.31-7.20 (m, 4H), 7.18 (s, 1H), 7.05 (s, 2H), 6.85 (d, J=11.0 Hz, 1H), 6.82-6.76 (m, 1H), 6.72 (d, J=8.5 Hz, 1H), 6.59 (s, 4H), 6.40 (d, J=2.2 Hz, 1H), 6.26 (d, J=2.2 Hz, 1H), 6.01 (d, J=11.8 Hz, 1H), 5.91 (s, 1H), 5.84 (s, 1H), 5.78 (d, J=7.8 Hz, 1H), 5.66-5.59 (m, 1H), 5.41 (d, J=7.1 Hz, 1H), 5.37 (d, J=7.8 Hz, 1H), 5.30 (dd, J=9.3, 3.8 Hz, 1H), 5.25-5.09 (m, 3H), 4.95 (s, 1H), 4.70 (q, J=6.2 Hz, 1H), 4.54-4.46 (m, 1H), 4.43 (d, J=5.3 Hz, 1H), 4.32 (q, J=7.5 Hz, 1H), 4.29-4.22 (m, 1H), 4.14-4.02 (m, 2H), 3.99-3.91 (m, 1H), 3.78-3.69 (m, 1H), 3.67 (s, 2H), 3.59 (t, J=8.5 Hz, 1H), 3.54 (d, J=8.7 Hz, 1H), 3.51-3.46 (m, 2H), 3.29 (s, 1H), 3.15-3.05 (m, 2H), 2.64 (s, 3H), 2.15 (s, 2H), 1.88-1.84 (m, 1H), 1.81-1.73 (m, 1H), 1.73-1.69 (m, 1H), 1.66-1.54 (m, 3H), 1.51 (s, 3H), 1.49-1.45 (m, 2H), 1.18-1.09 (m, 3H), 0.96-0.89 (m, 3H), 0.86 (dd, J=6.4, 2.5 Hz, 3H). ESI-TOF HRMS m/z 1803.5951 ([M+H]⁺, [C₈₅H₉₇Cl₃N₁₄O₂₄+H]⁺ requires 1803.5938).

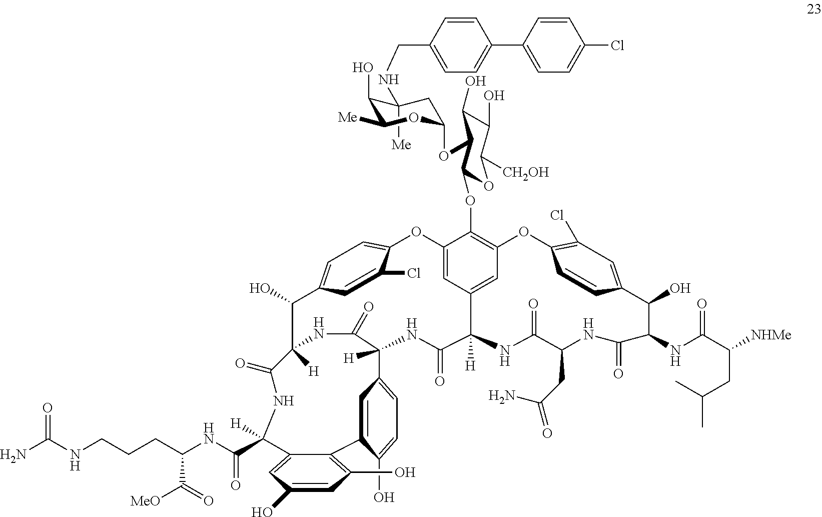

Compound 23 (7.5 mg, 55%, t_{R}=14.8 minutes; >95% HPLC purity): ¹H NMR (600 MHz, DMSO-d₆) δ 9.36 (s, 1H), 9.18 (s, 1H), 9.00 (s, 1H), 8.96 (s, 1H), 8.93 (s, 1H), 8.73 (s, 2H), 8.55 (s, 2H), 8.36 (d, J=7.4 Hz, 1H), 8.21 (s, 1H), 7.89 (s, 1H), 7.76-7.74 (m, 2H), 7.74-7.72 (m, 2H), 7.64 (s, 1H), 7.58 (dd, J=8.1, 2.2 Hz, 3H), 7.56-7.53 (m, 3H), 7.48 (t, J=8.1 Hz, 2H), 7.34 (dd, J=8.4, 4.0 Hz, 1H), 7.26 (d, J=8.4 Hz, 1H), 7.23 (d, J=3.4 Hz, 1H), 7.09 (s, 1H), 6.83-6.76 (m, 2H), 6.75 (s, 1H), 6.72 (d, J=8.5 Hz, 1H), 6.57 (s, 3H), 6.40 (d, J=2.3 Hz, 1H), 6.34 (d, J=2.2 Hz, 1H), 6.02 (s, 1H), 5.96 (s, 2H), 5.83 (s, 1H), 5.78 (d, J=7.8 Hz, 1H), 5.65 (s, 1H), 5.61 (s, 1H), 5.44-5.36 (m, 3H), 5.30 (s, 1H), 5.23-5.16 (m, 3H), 4.95 (s, 1H), 4.71 (q, J=6.0 Hz, 1H), 4.54 (d, J=5.9 Hz, 1H), 4.48 (s, 1H), 4.32 (q, J=7.5 Hz, 1H), 4.23 (d, J=11.7 Hz, 2H), 4.13-4.02 (m, 3H), 4.02-3.95 (m, 2H), 3.76-3.71 (m, 1H), 3.68 (s, 3H), 3.62-3.57 (m, 1H), 3.29 (d, J=8.9 Hz, 2H), 2.97-2.91 (m, 2H), 2.65 (s, 3H), 2.20-2.15 (m, 2H), 1.90-1.82 (m, 1H), 1.78-1.68 (m, 2H), 1.68-1.62 (m, 2H), 1.62-1.55 (m, 2H), 1.52 (s, 1H), 1.48 (s, 2H), 1.43-1.35 (m, 2H), 1.15 (dd, J=6.4, 2.8 Hz, 3H), 0.92 (d, J=6.2 Hz, 3H), 0.87 (dd, J=6.5, 2.5 Hz, 3H). ESI-TOF HRMS m/z 1819.5785 ([M+H]⁺, [C₈₆H₉₇Cl₃N₁₂O₂₆+H]⁺ requires 1819.5775).

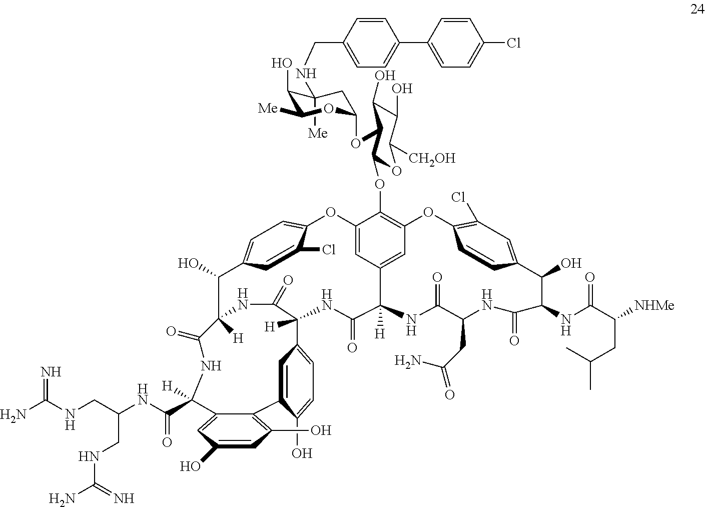

Compound 24 (4.4 mg, 29%, t_{R}=12.1 minutes; >95% HPLC purity): ¹H NMR (600 MHz, DMSO-d₆) δ 9.39 (s, 1H), 9.15 (s, 1H), 9.08 (s, 1H), 8.76 (s, 1H), 8.68 (s, 1H), 8.23 (d, J=7.6 Hz, 2H), 7.85 (s, 1H), 7.76-7.71 (m, 4H), 7.65 (s, 1H), 7.60-7.57 (m, 3H), 7.56-7.53 (m, 3H), 7.53-7.49 (m, 3H), 7.37 (d, J=8.2 Hz, 1H), 7.31 (s, 1H), 7.28 (d, J=8.2 Hz, 1H), 6.95 (d, J=10.9 Hz, 1H), 6.80 (d, J=8.5 Hz, 1H), 6.72 (d, J=8.5 Hz, 1H), 6.41 (d, J=2.3 Hz, 1H), 6.32 (d, J=2.2 Hz, 1H), 6.10 (s, 1H), 6.04 (s, 1H), 5.86 (s, 1H), 5.79 (d, J=7.8 Hz, 1H), 5.65 (s, 1H), 5.61 (s, 1H), 5.45-5.37 (m, 1H), 5.32-5.25 (m, 2H), 5.25-5.17 (m, 2H), 4.95 (s, 1H), 4.70 (t, J=6.4 Hz, 1H), 4.51 (s, 1H), 4.37 (d, J=4.9 Hz, 1H), 4.32 (d, J=11.6 Hz, 1H), 4.11 (d, J=7.2 Hz, 1H), 4.04-3.93 (m, 1H), 3.67 (s, 2H), 3.50 (s, 2H), 2.65 (s, 3H), 2.19-2.12 (m, 2H), 1.92-1.83 (m, 1H), 1.72-1.62 (m, 1H), 1.62-1.55 (m, 1H), 1.53 (s, 1H), 1.50 (s, 2H), 1.15 (d, J=6.2 Hz, 3H), 0.92 (d, J=6.2 Hz, 3H), 0.87 (dd, J=6.5, 2.7 Hz, 3H). ESI-TOF HRMS m/z 1803.5835 ([M+H]⁺, [C₈₄H₉₇Cl₃N₁₆O₂₃+H]⁺ requires 1803.6051).

### Synthesis of Compound 11

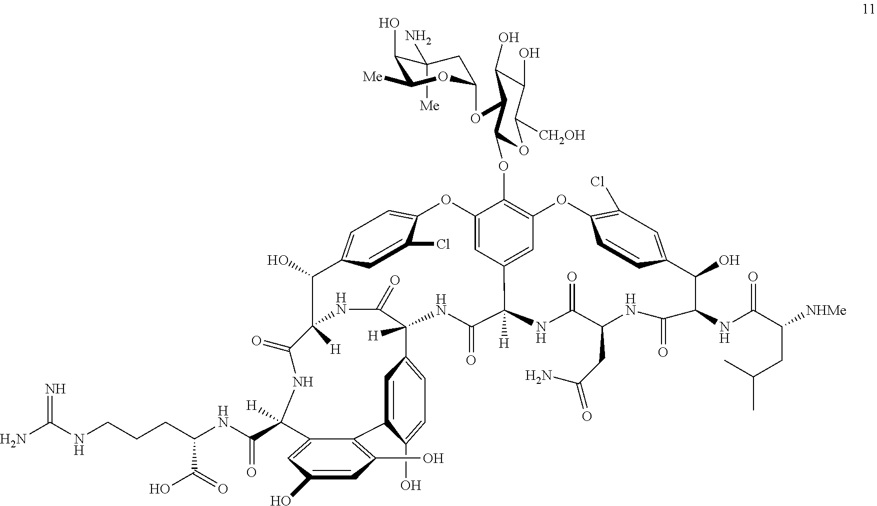

Compound 10 (5.0 mg, 2.5 μmol) in H₂O/THF (1/1, 250 μL) was treated with LiOH (0.6 mg, 25 μmol, 10 equiv) and the mixture was stirred at 25° C. for 30 minutes and quenched with the addition of H₂O (1 mL). THF was removed under a gentle stream of N₂ and the mixture was purified by semi-preparative reverse-phase HPLC (Nacalai Tesque, Inc., ARII-C18, 5 μm, 10×150 mm, 1-40% MeCN/H₂O-0.07% TFA gradient over 40 minutes, 3 mL/min, t_{R}=19.9 min) to afford Compound 11 (3.5 mg, 70%) as a white solid:

¹H NMR (600 MHz, DMSO-d₆) δ 9.27 (s, 1H), 8.97 (s, 2H), 8.94 (s, 1H), 8.69 (s, 1H), 8.57 (s, 1H), 8.32 (dd, J=8.3, 5.4 Hz, 1H), 7.89-7.82 (m, 1H), 7.64 (s, 3H), 7.56 (t, J=6.9 Hz, 1H), 7.53-7.44 (m, 2H), 7.39 (t, J=5.9 Hz, 1H), 7.34 (dd, J=8.3, 2.9 Hz, 1H), 7.27-7.17 (m, 2H), 7.06 (s, 1H), 6.85 (d, J=11.5 Hz, 1H), 6.80-6.74 (m, 1H), 6.71 (d, J=8.5 Hz, 1H), 6.56 (s, 4H), 6.42-6.36 (m, 2H), 5.98 (dd, J=9.3, 4.1 Hz, 1H), 5.91 (t, J=5.2 Hz, 1H), 5.77 (d, J=7.9 Hz, 1H), 5.66-5.54 (m, 1H), 5.47 (t, J=8.8 Hz, 1H), 5.40-5.30 (m, 1H), 5.25 (d, J=8.5 Hz, 1H), 5.23-5.18 (m, 2H), 5.18-5.15 (m, 1H), 5.13 (s, 1H), 4.93 (s, 1H), 4.75-4.63 (m, 1H), 4.57-4.50 (m, 1H), 4.46 (d, J=5.5 Hz, 1H), 4.28 (td, J=8.4, 5.5 Hz, 1H), 4.26-4.15 (m, 2H), 4.07-3.95 (m, 1H), 3.94 (s, 1H), 3.80-3.66 (m, 1H), 3.64 (q, J=8.0 Hz, 1H), 3.57-3.50 (m, 1H), 3.27 (s, 2H), 3.18 (s, 1H), 3.12-3.01 (m, 2H), 3.01-2.96 (m, 1H), 2.64 (d, J=6.2 Hz, 3H), 2.20-2.09 (m, 1H), 1.95-1.86 (m, 1H), 1.83-1.76 (m, 1H), 1.76-1.70 (m, 1H), 1.70-1.60 (m, 3H), 1.60-1.53 (m, 1H), 1.53-1.46 (m, 2H), 1.29 (s, 2H), 1.27 (s, 1H), 1.07 (d, J=6.0 Hz, 3H), 0.91 (dd, J=6.3, 1.8 Hz, 3H), 0.86 (dd, J=6.5, 2.6 Hz, 3H). ESI-TOF HRMS m/z 1604.5327 ([M+H]⁺, [C₇₂H₈₇Cl₂N₁₃O₂₅+H]⁺ requires 1604.5386).

### Synthesis of Compound 21

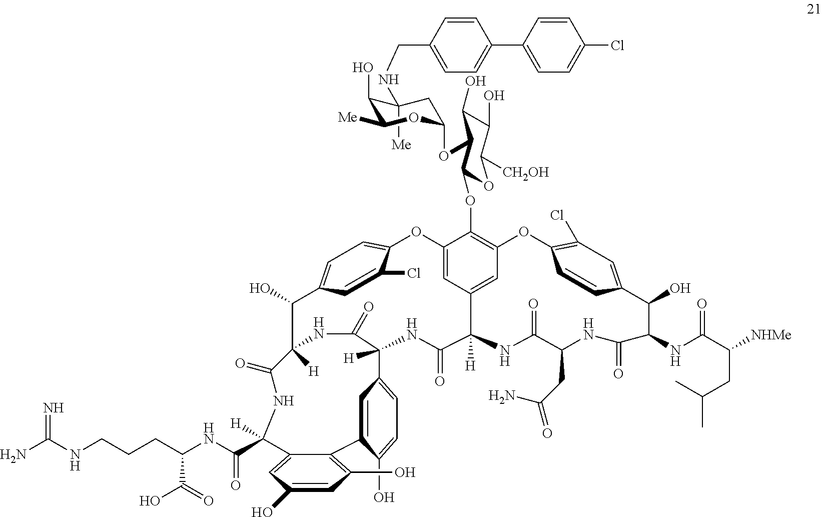

Compound 20 (2.5 mg, 1.25 μmol) in H₂O/THF (1/1, 125 μL) was treated with LiOH (0.3 mg, 12.5 μmol, 10 equiv) and the mixture was stirred at 25° C. for 30 minutes and quenched with the addition of H₂O (1 mL). THF was removed under a gentle stream of N₂ and the mixture was purified by semi-preparative reverse-phase HPLC (Nacalai Tesque, Inc., ARII-C18, 5 μm, 10×150 mm, 20-80% MeCN/H₂O-0.07% TFA gradient over 40 minutes, 3 mL/min, t_{R}=13.3 minutes) to afford Compound 21 (2.3 mg, 92%) as a white solid:

¹H NMR (600 MHz, DMSO-d₆) δ 9.28 (s, 1H), 8.99 (s, ¹H), 8.96 (s, 1H), 8.67 (s, 1H), 8.58 (s, 1H), 8.27 (s, 1H), 7.87 (s, 1H), 7.77-7.69 (m, 3H), 7.69-7.61 (m, 1H), 7.61-7.56 (m, 2H), 7.54 (d, J=8.2 Hz, 2H), 7.52-7.43 (m, 3H), 7.33 (d, J=8.5 Hz, 2H), 7.23 (s, 1H), 6.85 (d, J=10.7 Hz, 1H), 6.77 (d, J=8.5 Hz, 1H), 6.71 (d, J=8.5 Hz, 1H), 6.39 (s, 1H), 6.36 (s, 1H), 5.95 (s, 1H), 5.82 (s, 1H), 5.77 (s, 1H), 5.67-5.55 (m, 1H), 5.37 (d, J=5.3 Hz, 1H), 5.29 (d, J=9.3 Hz, 1H), 5.22-5.12 (m, 3H), 4.93 (s, 1H), 4.74-4.64 (m, 1H), 4.49 (d, J=12.0 Hz, 2H), 4.24 (s, 2H), 4.12 (t, J=5.4 Hz, 1H), 4.04 (s, 1H), 3.75-3.64 (m, 2H), 3.64-3.56 (m, 2H), 3.11-3.04 (m, 2H), 2.61 (s, 3H), 2.18-2.10 (m, 1H), 1.91-1.81 (m, 1H), 1.81-1.74 (m, 1H), 1.71-1.61 (m, 2H), 1.51 (s, 3H), 1.47 (s, 1H), 1.13 (d, J=6.1 Hz, 3H), 0.91 (d, J=6.1 Hz, 3H), 0.86 (d, J=6.1 Hz, 3H). ESI-TOF HRMS m/z 1804.5828 ([M+H]⁺, [C₈₅H₉₆Cl₃N₂₃O₂₅+H]⁺ requires 1804.5784).

### Synthesis of Compound 27

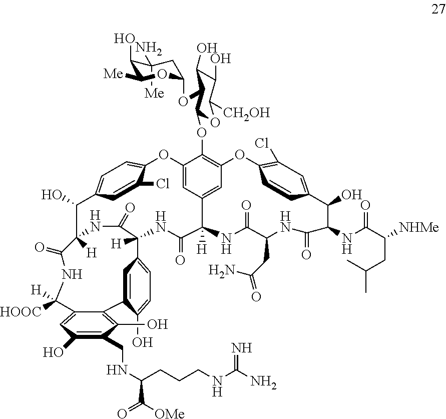

A solution of vancomycin hydrochloride (10.0 mg, 6.7 μmol) and L-arginine methyl ester dihydrochloride (13.6 mg, 50 μmol, 7.5 equiv) in H₂O (50 μL) was treated sequentially with Na₂CO₃ solution (3 M in H₂O, 25 μL) and formaldehyde (37% in H₂O, 0.5 μL, 6.7 μmol, 1 equiv). The mixture was stirred at 5° C. for 16 hours and quenched with the addition of H₂O (1 mL). The mixture was purified by semi-preparative reverse-phase HPLC (Nacalai Tesque, Inc., ARII-C18, 5 μm, 10×150 mm, 1-40% MeCN/H₂O-0.07% TFA gradient over 40 minutes, 3 mL/min, t_{R}=20.4 minutes) to afford 27 (4.4 mg, 31%) as a white solid:

¹H NMR (600 MHz, DMSO-d₆) δ 9.24 (s, 1H), 9.08 (s, ¹H), 8.98 (s, 1H), 8.79 (s, 1H), 8.64 (s, 1H), 7.84 (s, 1H), 7.65 (s, 3H), 7.53 (s, 2H), 7.47 (d, J=8.4 Hz, 1H), 7.34 (d, J=8.3 Hz, 1H), 7.22 (d, J=8.1 Hz, 1H), 7.13 (s, 1H), 6.86 (s, 1H), 6.81-6.71 (m, 2H), 6.61-6.31 (m, 1H), 5.98 (d, J=4.3 Hz, 2H), 5.75 (d, J=7.4 Hz, 1H), 5.71 (s, 1H), 5.47 (d, J=6.5 Hz, 1H), 5.37 (s, 1H), 5.26 (s, 2H), 5.14 (s, 3H), 4.69 (q, J=6.7 Hz, 1H), 4.45 (t, J=8.5 Hz, 2H), 4.14 (s, 2H), 4.08-3.97 (m, 2H), 3.75 (s, 2H), 3.18 (d, J=5.6 Hz, 2H), 3.10 (s, 3H), 2.61 (s, 3H), 2.21-2.11 (m, 1H), 2.02 (d, J=4.9 Hz, 1H), 1.95-1.86 (m, 1H), 1.73 (d, J=12.9 Hz, 1H), 1.70-1.61 (m, 2H), 1.61-1.50 (m, 2H), 1.50-1.43 (m, 1H), 1.31 (m, 3H), 1.07 (d, J=6.1 Hz, 3H), 0.93 (d, J=5.9 Hz, 3H), 0.88 (d, J=6.0 Hz, 3H). ESI-TOF HRMS m/z 1648.5629 ([M+H]⁺, [C₇₄H₉₂Cl₂N₂₃O₂₆+H]⁺ requires 1648.5648).

### Synthesis of Compound 28

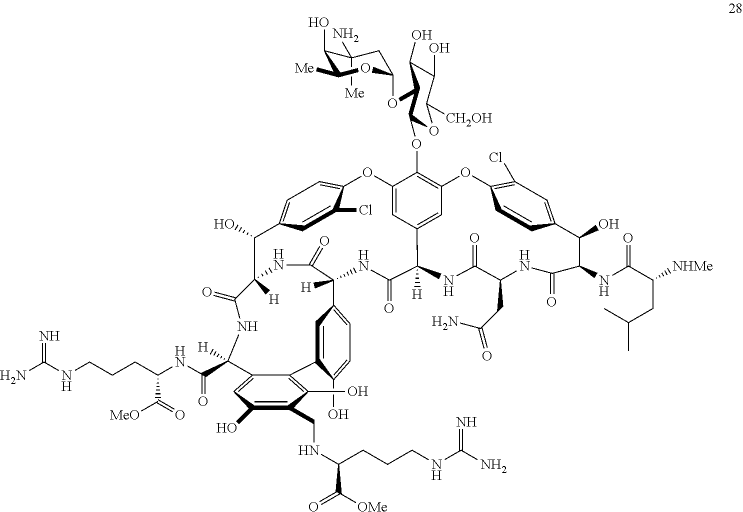

Compound 27 (3.9 mg, 2.0 μmol) in DMF/DMSO (1/1, 200 μL) was treated sequentially with L-arginine methyl ester dihydrochloride (1.0 mg, 4.0 μmol, 2 equiv), N-methylmorpholine (distilled, 1 M in DMF/DMSO=1/1, 12 μL, 12 μmol, 6 equiv), and HBTU (0.5 M in DMF/DMSO=1/1, 8 μL, 4.0 μmol, 2 equiv). The mixture was stirred at 5° C. for 16 hours and quenched with the addition of H₂O (1 mL). The mixture was purified by semi-preparative reverse-phase HPLC (Nacalai Tesque, Inc., ARII-C18, 5 μm, 10×150 mm, 1-40% MeCN/H₂O-0.07% TFA gradient over 40 min, 3 mL/min, t_{R}=19.7 minutes) to afford 28 (2.6 mg, 54%) as white solid:

¹H NMR (600 MHz, DMSO-d₆) δ 10.27 (s, 1H), 9.34 (s, ¹H), 9.25 (s, 1H), 9.03 (s, 1H), 8.95 (s, 1H), 8.79 (s, 2H), 8.60 (s, 1H), 8.43 (s, 1H), 7.84 (d, J=6.4 Hz, 1H), 7.66 (s, 4H), 7.52 (t, J=9.8 Hz, 1H), 7.49-7.44 (m, 2H), 7.32 (d, J=8.3 Hz, 1H), 7.28 (d, J=8.3 Hz, 1H), 7.23 (d, J=8.3 Hz, 1H), 7.18 (s, 1H), 6.98 (s, 1H), 6.93 (s, 1H), 6.85 (s, 1H), 6.79 (s, 1H), 6.62 (s, 1H), 5.98 (s, 1H), 5.94 (s, 1H), 5.75 (d, J=7.1 Hz, 1H), 5.72-5.62 (m, 1H), 5.47 (s, 1H), 5.33 (d, J=7.3 Hz, 1H), 5.29-5.19 (m, 2H), 5.16 (d, J=11.9 Hz, 2H), 5.13 (s, 1H), 4.83 (s, 1H), 4.68 (q, J=7.9 Hz, 1H), 4.54 (d, J=5.9 Hz, 1H), 4.48 (s, 1H), 4.41 (q, J=7.9 Hz, 1H), 4.16 (s, 3H), 4.09-3.99 (m, 3H), 3.97-3.78 (m, 3H), 3.74 (s, 4H), 3.67 (s, 3H), 3.66-3.61 (m, 2H), 3.54 (d, J=8.4 Hz, 2H), 3.27 (s, 2H), 3.18 (s, 2H), 3.11-3.06 (m, 3H), 3.06-3.00 (m, 2H), 2.59 (s, 3H), 2.17-1.99 (m, 1H), 1.90 (d, J=9.3 Hz, 1H), 1.84-1.73 (m, 1H), 1.73-1.69 (m, 1H), 1.68-1.62 (m, 2H), 1.54-1.47 (m, 3H), 1.30 (s, 2H), 1.27 (s, 1H), 1.10-1.03 (m, 3H), 0.93 (d, J=5.8 Hz, 3H), 0.87 (d, J=6.0 Hz, 3H). ESI-TOF HRMS m/z 1818.6778 ([M+H]⁺, [C₈₁H₁₀₅Cl₂N₁₇O₂₇+H]⁺ requires 1818.6815).

### II. In Vitro Antimicrobial Assays⁵⁷

One day before experiments were run, fresh cultures of vancomycin-sensitive Staphylococcus aureus (VSSA strain ATCC 25923), methicillin and oxacillin-resistant Staphylococcus aureus (MRSA strain ATCC 43300), vancomycin-resistant Enterococcus faecalis (VanA VRE, BM4166 and ATCC BAA-2573), Enterococcus faecium (VanA VRE, ATCC BAA-2317 and TX-2465), vancomycin-resistant Enterococcus faecalis (VanB VRE, strain ATCC 51299), Escherichia coli (ATCC 25922), Acinetobacter baumannii (ATCC BAA-1710), Pseudomonas aeruginosa (ATCC 15442), Klebsiella pneumoniae (ATCC 700603) were inoculated and grown in an orbital shaker at 37° C. in 100% Mueller-Hinton broth (VSSA, MRSA and VanB VRE), 100% brain-heart infusion broth (VanA VRE, A. baumannii and K. pneumoniae) or 100% Luria broth (E. coli and P. aeruginosa). After 24 hours, the bacterial stock solutions were serial diluted with the culture medium (10% Mueller-Hinton broth for VSSA, MRSA and VanB VRE or 10% brain-heart infusion broth for VanA VRE A. baumannii and K. pneumoniae or 10% Luria Broth for E. coli and P. aeruginosa, containing 0.002% Tween®-80) to achieve a turbidity equivalent to a 1:100 dilution of a 0.5 M McFarland solution. This diluted bacterial stock solution was then inoculated in a 96-well flat-bottom non-treated microtiter plate (Corning 3370), supplemented with serial diluted aliquots of the antibiotic solution in DMSO (4 μL), to achieve a total assay volume of 0.1 mL.

The plate was then incubated at 37° C. for 18 hours, after which minimal inhibitory concentrations (MICs) were determined by monitoring the cell growth (observed as a pellet) in the wells. The lowest concentration of antibiotic (in μg/mL) capable of eliminating cell growth in the wells is the reported MIC value. The reported MIC values for the vancomycin analogues were determined against vancomycin as a standard in the first well.

Although all of the analogues tested exhibited slightly less potency against VanA E. faecalis (BM 4166) compared with VanA E. faecium (ATCC BAA-2317), the same results were observed where the equimolar mixtures of the singly modified vancomycins did not display the enhanced potency found in G3-CBP-vancomycin (Compound 15) and GBn-CBP-vancomycin (Compound 19), exhibiting antimicrobial activity only at the level of the most potent compound in the mixture (CBP-vancomycin, Compound 2).

### III. Site Specificity of Guanidinium-Modified Analogues

To investigate if the guanidinium modification on vancomycin is site-specific as was the trimethylammonium cation modification, analogue Compound 27 bearing a guanidinium modification on vancomycin A-ring and analogue Compound 28 bearing guanidinium modifications on both the A-ring and at the C-terminus were synthesized. The antimicrobial activity of each of these analogues was established against vancomycin-resistant organisms (VRE, VanA, 4 strains). The results are shown below in Table 10 alongside vancomycin and the earlier trimethylammonium cation modified analogues⁵⁵.

TABLE 10
Antimicrobial activity of Compounds 1, 3, 10 and 27-30, MIC(μg/mL)³⁷

| | | VanA | VanA | |
|---|---|---|---|---|
| | VanA | E. faecium | E. faecalis | VanA |
| | E. faecalis | ATCC | ATCC | E. faecium |
| Compound | BM 4166 | BAA-2317 | BAA-2573 | TX 2465 |
| vancomycin (1) | 250 | 250 | 125 | 250 |
| Arg(OMe)-vancomycin (10) | 8 | 8 | 4 | 8 |
| Arg(OMe)^{Ar}-vancomycin (27) | 31 | 8 | 16 | 63 |
| Arg(OMe)-Arg(OMe)^{Ar}- | 8 | 2 | 1 | 8 |
| vancomycin (28) | | | | |
| C1-vancomycin (3) | 63 | 31 | N.D. | N.D. |
| C1^{Ar}-vancomycin (29) | 125 | 31 | N.D. | N.D. |
| C1-C1^{Ar}-vancomycin (30) | 16 | 4 | N.D. | N.D. |

As is seen, compared with Arg(OMe)-vancomycin (Compound 10), Arg(OMe)^{Ar}-vancomycin (Compound 25) bearing the same modification on A-ring is as much as 8-fold less potent against the resistant organisms. This observation is consistent with previous results on the site-specific nature of the permanent positive charge modification on vancomycin. Arg(OMe)-Arg(OMe)^{AR}-vancomycin (Compound 26) bearing both a C-terminus and an A-ring guanidinium modification exhibited a potency essentially equal to that of Arg(OMe)-vancomycin (Compound 10), but not significantly further enhanced against vancomycin-resistant organisms. Similarly, guanidinium vancomycin analogue Compounds 27 and 28 were also found to be about 2 to 4-fold more potent than corresponding trimethylammonium modified vancomycin analogue Compounds 29 and 30 against the VanA VRE strains.

### IV. Resistance Development Assay, Durability⁶⁶

G3-vancomycin (Compound 5), G3-CBP-vancomycin (Compound 15) and other front-line antibiotics (daptomycin, linezolid and tigecycline) were examined against vancomycin-resistant Enterococcus faecalis (VanA VRE, BM4166) and Enterococcus faecium (VanA VRE, ATCC BAA-2317). One day before studies were carried out, fresh cultures of vancomycin-resistant Enterococcus faecalis (VanA VRE, BM 4166) and Enterococcus faecium (VanA VRE, ATCC BAA-2317) were inoculated and grown in an orbital shaker at 37° C. in 100% brain-heart infusion broth. After 24 hours, the bacterial stock solutions were serially diluted with the culture medium (10% brain-heart infusion broth, containing 0.002% Tween®-80) to achieve a turbidity equivalent to a 1:100 dilution of a 0.5 M McFarland solution (1.5×10⁶ CFU/mL).

The resulting diluted bacterial stock solution was then inoculated in a 96-well flat-bottom non-treated microtiter plate (Corning 3370), supplemented with antibiotic solution in DMSO (4 μL), to achieve a total assay volume of 100 μL. The plate was then incubated at 37° C. for 18 hours, after which minimum inhibitory concentrations (MICs) were determined by monitoring the cell growth (observed as a pellet) in the wells. The lowest concentration of antibiotic (in μg/mL) capable of eliminating cell growth in the wells is the reported MIC value.

The bacterial suspensions (40 μL) in the 96-well plate at sub-MIC concentrations of Compounds 5 and 15 (0.5×MIC) were inoculated with 100% brain-heart infusion broth and the bacteria were grown in an orbital shaker at 37° C. for 6 hours until the value of OD₆₀₀ became 0.6.

A new MIC assay was performed with the same protocol above. This process was repeated for 50 passages, and the fold increase in MIC was determined after each passage.

### V. Permeability Assay⁵⁹⁻⁶⁰

One day before studies were carried out, cultures of vancomycin-resistant Enterococcus faecalis (VanA VRE, BM 4166) and Enterococcus faecium (VanA VRE, ATCC BAA-2317) were inoculated and grown in an orbital shaker at 37° C. in 100% brain-heart infusion broth for 12 hours. The above bacterial solution was subjected to a subculture to obtain fresh mid-log phase bacterial cells (incubation time=6 hours). The bacterial suspension was diluted to a total volume of 7 mL with OD₆₀₀=0.6.

After the cultured bacteria were harvested (3000 rpm, 4° C., 20 minutes), the white bacterial precipitate was washed and resuspended in 5 mM glucose and 5 mM HEPES buffer (1:1, 5.00 mL, pH=7.2). This bacterial suspension (130 μL) was charged in a 96-well black plate with a clear bottom (Corning 3651). The propidium iodide dye (10 μL, 150 μM DMSO solution) was added to the above suspension and the fluorescence was monitored at 25° C. for 5 minutes at 20 or 30 second intervals using a microplate reader (Molecular Devices®, Max Gemini EX) at an excitation wavelength of 535 nm and an emission wavelength of 617 nm. The antibiotic solution (10 μL, 150 μM buffer solution) was added to the cell suspension and the fluorescence was monitored at 25° C. for an additional 15 minutes.

### VI. Impact of Lipoteichoic Acid (LTA) on In Vitro Antimicrobial Activity of Vancomycin Analogues⁶¹

One day before studies were run, fresh cultures of vancomycin-resistant Enterococcus faecalis (VanA VRE, BM 4166) and Enterococcus faecium (VanA VRE, ATCC BAA-2317) were inoculated and grown in an orbital shaker at 37° C. in 100% brain-heart infusion broth. After 24 hours, the bacterial stock solutions were serially diluted with the culture medium (10% brain-heart infusion broth containing 0.002% Tween®-80 with 100 μg/mL lipoteichoic acid (Sigma-Aldrich, from S. aureus) as study group and 10% brain-heart infusion broth containing 0.002% Tween®-80 as control group) to achieve a turbidity equivalent to a 1:100 dilution of a 0.5 M McFarland solution (1.5×10⁶ CFU/mL). This diluted bacterial stock solution was then inoculated in a 96-well flat-bottom non-treated microtiter plate (Corning 3370), supplemented with serial diluted aliquots of the antibiotic solution in DMSO (4 μL), to achieve a total assay volume of 100 μL.

The resulting plate was then incubated at 37° C. for 18 hours, after which minimum inhibitory concentrations (MICs) were determined by monitoring the cell growth (observed as a pellet) in the wells. The lowest concentration of antibiotic (in μg/mL) capable of eliminating cell growth in the wells is the reported MIC value.

The reported MIC values for the vancomycin analogues were determined against vancomycin as a standard in the first well. When NaH₂PO₄ or POPE was used instead of LTA, 10% brain-heart infusion broth containing 0.002% Tween®-80 with 100 μg/mL NaH₂PO₄ or 100 μg/mL POPE was used as the culture medium used for serial dilution. When additional G3 (H₂N⁺(CH₂)₃NHC(NH₂)═NH₂⁺.2TFA) or Mg²⁺ was used, 10% brain-heart infusion broth containing 0.002% Tween®-80 with 100 μg/mL G3 or 100 μg/mL MgCl₂ and 100 μg/mL lipoteichoic acid was used as the culture medium for serial dilution of study group, 10% brain-heart infusion broth containing 0.002% Tween®-80 with 100 μg/mL G3 was used as the culture medium for serial dilution of control group.

### VII. Impact of Lipoteichoic Acid (LTA) on Bacterial Cell Membrane Permeability Induced by G3-CBP-Vancomycin (Compound 15)

One day before studies were carried out, cultures of vancomycin-resistant Enterococcus faecalis (VanA VRE, BM 4166) and Enterococcus faecium (VanA VRE, ATCC BAA-2317) were inoculated and grown in an orbital shaker at 37° C. in 100% brain-heart infusion broth for 12 hours. The above bacterial solution was subjected to a subculture to obtain fresh mid-log phase bacterial cells (incubation time=6 hours). The bacterial suspension was diluted to a total volume of 7 mL with OD₆₀₀=0.6.

After the cultured bacteria were harvested (3000 rpm, 4° C., 20 minutes), the white bacterial precipitate was washed and resuspended in 5 mM glucose and 5 mM HEPES buffer (1:1, 5.00 mL, pH=7.2) with lipoteichoic acid (100 μg/mL), or in 5 mM glucose and 5 mM HEPES buffer (1:1, 5.00 mL, pH=7.2) as control. This bacterial suspension (130 μL) was charged in a 96-well black plate with a clear bottom (Corning 3651). The propidium iodide dye (10 μL, 150 μM DMSO solution) was added to the above suspension and the fluorescence was monitored at 25° C. for 5 minutes at 20 or 30 second intervals using a microplate reader (Molecular Devices®, Max Gemini EX) at an excitation wavelength of 535 nm and an emission wavelength of 617 nm. The antibiotic solution (10 μL, 150 μM buffer solution) was added to the cell suspension and the fluorescence was monitored at 25° C. for an additional 15 minutes.

### VIII. Hemolysis Assay⁶²

Following an established procedure and as previously reported, the blood cells in pig whole blood (2 mL, Pel-Freez Biologicals, non-sterile, sodium citrate) were harvested (3000 rpm, 4° C., 20 minutes), and the red blood precipitate was washed and resuspended in phosphate buffered saline (pH 7.4). This diluted red blood cell stock solution (384 μL) was incubated with the compound solution in DMSO (16 μL) in a 1 mL microtube to achieve the final concentration of the assayed compounds. The mixture was then incubated at 37° C. for 1 hour.

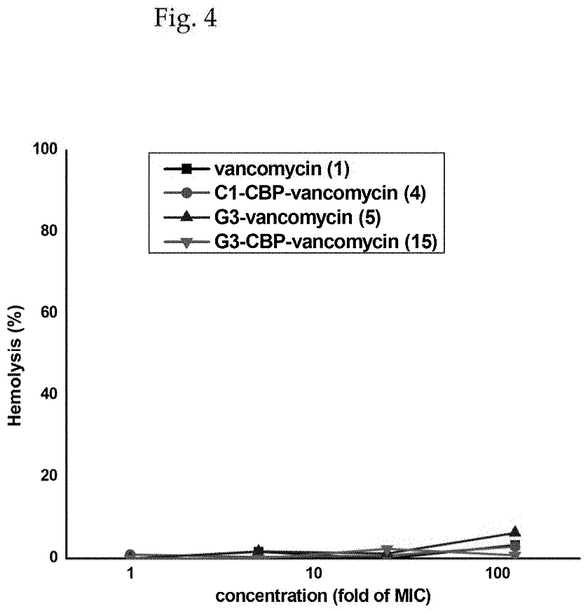

The incubated solution was diluted with phosphate buffered saline (pH 7.4, 200 μL) at 25° C. and centrifuged (3000 rpm, 4° C., 20 minutes). The supernatant (200 μL) was transferred to a microtiter plate. A positive control (0.2% vol % Triton® X-100, 100% total hemolysis) and the negative control (no antibiotic, 0% hemolysis) were prepared. A₃₅₀ was measured using a microplate reader (Molecular Devices®, Max Gemini EX). The percent hemolysis was determined by equation 1, below, and the results are presented in FIG. 4.

$$\begin{matrix}Hemolysis ( \% ) = \frac{( {A}_{test} - {A}_{zero} )}{( {A}_{total} - {A}_{zero} )} \times 100 & \begin{matrix}{A}_{test} : Absorbance with test compound \\ {A}_{total} : Absorbance of 100 \% hemolysis \\ {A}_{zero} : Absorbance of 0 \% hemolysis\end{matrix}\end{matrix} \qquad ( eq . 1 )$$

Although the differences in mammalian and bacterial cell wall composition are extensive, including the more highly anionic composition of the bacterial cell wall responsible for a preferential and differential cation binding, lysis of mammalian cell membranes (red blood cells) are potential off-target consequences of cationic compounds that impact bacterial cell membrane integrity. The standard red blood cell hemolysis assay was conducted and measures the extent of red blood cell lysis after 1 hour exposure to the compound (pH 7.4, PBS, 37° C., 1 hour). No compound in the present series exhibited any hemolytic activity even at concentrations >100-fold above their MICs.

### IX. PK Studies, Mouse Pharmacokinetics

Pharmacokinetic (PK) behavior of G3-CBP-vancomycin and C1-CBP-vancomycin were examined in 7-8 week-old female CD-1 mice by intravenous dosing utilizing the tail vein. Test compounds were formulated v:v:v in 10% DMSO:15% PEG-400:75% (30% w:v hydroxypropyl β-cyclodextrin in water) by first dissolving in DMSO then adding PEG-400 and the 30% w:v solution of hydroxypropyl β-cyclodextrin. Formulation solutions of 2 or 10 mg/mL were used for 10 and 50 mg/kg PK studies, respectively.

Triplicate mice were dosed and 20 μL blood was collected into Li-heparin-coated hematocrit tubes 5, 15, 30, 60, 120, 240, 360, 480, and 1440 minutes after dosing. Plasma was obtained by centrifugation using a microcentrifuge fit with a hematocrit rotor, and obtained plasma was immediately frozen. The use of micro-sampling blood collection reduces the total blood taken from the mouse and allows a single mouse to be used for the entire time course, reducing inter-individual variability and maintaining the health of the mouse.

Drug levels were determined by mass spectrometry using an ABSciex 5500 mass spectrometer using multiple reaction monitoring. G3-CBP-vancomycin and C1-CBP-vancomycin were detected as their 3+ charged molecular ions. Pharmacokinetic parameters were calculated using a non-compartmental model (Phoenix WinNonlin, Pharsight Inc.), and are shown in Table 11, below. All PK work was conducted in the Scripps Florida vivarium which is fully AAALAC accredited. Procedures were approved by the Scripps Florida IACUC, protocol number 15-022.

TABLE 11
Comparison PK properties of Compound 15 with those of Compounds
2, 4, and vancomycin established in an earlier study^{38, a}

| | vancomycin | | CBP-vancomycin (2) | | C1-CBP-vancomycin (4) | | | G3-CBP-vancomycin (15) | |
|---|---|---|---|---|---|---|---|---|---|
| Parameter | 300 mg/kg | 10 mg/kg | 75 mg/kg | 10 mg/kg | 50 mg/kg | 10 mg/kg | | 50 mg/kg | 10 mg/kg |
| Cₘₐₓ (μg/mL) | 1665 | 62.8 | 125 | 65.0 | 58.9 | 14.1 | (14.5)^{b} | 152 | 35.9 |
| tₘₐₓ (h) | 0.083 | 0.083 | 0.25 | 0.083 | 0.25 | 0.50 | (0.08) | 0.28 | 0.14 |
| AUC (μg-h/mL) | 935 | 21.8 | 575 | 135 | 430 | 81.1 | (48.4) | 312 | 66.0 |
| V_{d} (L/kg) | 0.62 | 0.34 | 1.24 | 1.28 | 1.04 | 1.25 | (0.64) | 0.35 | 0.41 |
| CL (L/h/kg) | 0.32 | 0.46 | 0.13 | 0.074 | 0.12 | 0.12 | (0.12) | 0.09 | 0.09 |
| t_{1/2} (h) | 1.35 | 0.52 | 6.6 | 12.0 | 6.2 | 7.0 | (5.1) | 4.4 | 4.3 |

^{a}Compounds administered iv @ MTD and 10 mg/kg in mice (n = 3/time point, measured at 0.083, 0.25, 0.5, 1, 2, 4, 6, 8 and 24 hours).
^{b}Values in parentheses were determined in this study.

### X. In Vivo Antimicrobial Efficacy Study

Neutropenic ICR (CD-1) mice, age 5-6 weeks weighing 22±2 g, were used for this study.

Cyclophosphamide (CP) was administered by two intraperitoneal (ip) injections to induce neutropenia following a standard method for mouse thigh infection models. The first CP dose (150 mg/kg) was administered 4 days before infection (day −4) and the second (100 mg/kg) was given 24 hours (day −1) prior to infection on day 0. This cyclophosphamide treatment schedule results in neutropenia (<100 neutrophils/mL) until day 2 after infection.

On day 0, animals were infected with the pathogen suspension (0.1 mL, 1.01×10⁵ CFU/mL) by intramuscular injection into the right thigh. The challenging multidrug resistant and vancomycin-resistant S. aureus strain (VanA VRSA, VRS2)⁵⁰ was used. Compound 15 alongside linezolid were administered to animals (n=5 for each dose) by subcutaneous (sc, Compound 15) or oral (po, linezolid) administration using doses defined by the MTD/PK studies and given as single dose 2 hours following injection of the pathogen. This was a single dose given at 12.5, 25, 50 and 100 mg/kg (for Compound 15), at 50 mg/kg (for reference standard linezolid, po administration twice at 2 and 12 hours) and at 0 (vehicle control).

Vehicle was 10% DMSO, 15% PEG-400, 22.5% hpbCD in water and the dose volume was 10 mL/kg for all test groups. Linezolid was dissolved in water for injection with 1% Tween®-80 for oral (po) administration. Animals were monitored for 30 minutes after dosing to detect acute toxicity and were checked periodically after infection for humane endpoints. Animals were sacrificed with CO₂ asphyxiation at the scheduled time points for tissue harvest, at 2 hours (for baseline) or 26 hours after infection.

Thigh muscle tissue was aseptically harvested from each of the sacrificed animals, weighed, and homogenized in 3 mL sterile PBS (pH 7.4) with a polytron homogenizer. Bacterial burden in the tissue homogenates was determined by performing 10-fold serial dilutions and plating 0.1 mL of each to nutrient agar (NA) plates. Colonies were counted after 18-24 hours incubation. The colony forming units per gram tissue (CFU/g) were calculated.

For each animal, the following raw data is recorded and tabulated: tissue weight and bacterial counts in each tissue homogenate dilution. For each tissue, the homogenate dilution that yielded the largest number of colonies, between 10 to 300 colonies per plate, was selected to calculate the bacterial counts per gram of tissue (CFU/g). Bacterial counts per gram of tissue (CFU/g) were calculated and tabulated and plotted in GraphPad Prism. The raw colony count data of the homogenate dilutions was inspected for proportionality within the dilution series.

The 10-fold serial dilutions are expected to show 10-fold reductions in counts. Disproportionate data, such as fewer counts in the undiluted homogenate samples compared to the diluted sample, would indicate inhibition of colony growth due to drug carry over from the thigh tissue to the test plate. Aberrant titration data was not observed. Data were plotted as the bacterial counts per gram tissue of control and treatment groups to assess the dose responsive effects.

The difference in bacterial density between the baseline group (2 hours initial counts) and the treatment group was also calculated and plotted: Δ=CFU/g of treatment−CFU/g of baseline. The doses that result in a net static effect relative to baseline, and those that result in 1-log₁₀, 2-log₁₀ or more bactericidal effects were determined.

Inoculum Preparation.

A 0.2 mL aliquot of a single-use glycerol stock of the pathogen (at −80° C.) was used to seed 20 mL of brain-heart infusion (BHI) broth and then incubated at 35-37° C. with shaking (250 rpm) for 8 hours. Cells in the 20 mL culture were pelleted by centrifugation (3,500×g) for 15 min, and then resuspended in 10 mL cold phosphate buffer saline (PBS). The optical density, OD_{620 nm}, was measured and used to guide dilution.

The PBS suspensions were stored on ice for no more than one hour prior to animal inoculation. Bacterial count in the challenge organism suspension was enumerated by dilution plating to NA plates followed by 20-24 h incubation. The target inoculum was 1×10⁶ CFU/mL and actual CFU count was 1.24×10⁵ CFU/mL. These studies were performed by Pharmacology Discovery Services Taiwan, Ltd., a partner lab of Eurofins Pharma Discovery Services.

### CITATIONS

1. McCormick, Antibiot. Annu. 1956, 3, 606-611.
2. Perkins, Pharmacol. Ther. 1982, 16, 181-197.
3. Van Bambeke et al., Drugs 2004, 64, 913-936.
4. Levine, Clin. Infect. Dis. 2006, 42, S5-S12.
5. Kahne et al., Chem. Rev. 2005, 105, 425-448.
6. Barna et al., Annu. Rev. Microbiol. 1984, 38, 339-357.
7. Hubbard et al., Angew. Chem., Int. Ed. 2003, 42, 730-765.
8. Williams et al., Angew. Chem., Int. Ed. 1999, 38, 1172-1193.
9. Perkins, Pharmacol. Ther. 1982, 16, 181-197.
10. Leclercq et al., N. Engl. J. Med. 1988, 319, 157-161.
11. Pootoolal et al., Annu. Rev. Pharmacol. Toxicol. 2002, 42, 381-408.
12. Courvalin, Clin. Infect. Dis. 2006, 42, S25-S34.
13. Weigel et al., Science 2003, 302, 1569-1571.
14. Walsh et al., Annu. Rev. Microbiol. 2002, 56, 657-675.
15. Willyard, Nature 2017, 543, 15.
16. Walsh, et al., Science 1993, 261, 308-310.
17. Healy et al., Chem. Biol. 2000, 7, R109-R119.
18. McComas et al., J. Am. Chem. Soc. 2003, 125, 9314-9315.
19. Guan et al., J. Med. Chem. 2018, 61, 286-304.
20. Yarlagadda et al., J. Antibiot. 2015, 68, 302-312.
21. Yarlagadda et al., Angew. Chem., Int. Ed. 2016, 55, 7836-7840.
22. Yarlagadda et al., Angew. Chem., Int. Ed. 2015, 54, 13644-13649.
23. Long et al., J. Antibiot. 2008, 61, 603-614.
24. Yarlagadda et al., J. Med. Chem. 2014, 57, 4558-4568.
25. Leadbetter et al., J. Antibiot. 2004, 57, 326-336.
26. Corey et al., Nat. Rev. Drug Discov. 2009, 8, 929-930.
27. Anderson et al., Drugs 2008, 68, 639-648.
28. Markham, Drugs 2014, 74, 1823-1828.
29. Okano et al., Proc. Natl. Acad. Sci., U.

S. A. 2017, 114, E5052-E5061.
30. Okano et al., J. Am. Chem. Soc. 2015, 137, 3693-3704.
31. Okano et al., J. Am. Chem. Soc. 2014, 136, 13522-13525.
32. Crowley et al., J. Am. Chem. Soc. 2006, 128, 2885-2892.
33. Boger, J. Org. Chem. 2017, 82, 11961-11980.
34. Ge et al., Science 1999, 284, 507-511.
35. Kerns et al., J. Am. Chem. Soc. 2000, 122, 12608-12609.
36. Wu et al., ACS Infect. Dis. 2018, 4, 1468-1474.
37. Wu et al., Tetrahedron 2019, 75, 3160-3165.
38. Wu et al., J. Org. Chem. 2020, 85, 1365-1375.
39. Rajagopal et al., In Protein and Sugar Export and Assembly in Gram-positive Bacteria, Springer, Cham, 2015; pp 1-44.
40. Unpublished studies suggest vancomycin C-terminus amide formation, neutralizing the carboxylic acid negative charge, may modestly enhance activity against vancomycin-resistant organisms.
41. For VanA E. faecalis (VanA VRE, BM 4166): resistant to erythromycin, gentamicin, chloramphenicol, and ciprofloxacin as well as vancomycin and teicoplanin; sensitive to daptomycin. For VanA E. faecium (VanA VRE, ATCC BAA-2317): resistant to ampicillin, benzylpenicillin, ciprofloxacin, erythromycin, levofloxacin, nitrofurantoin, and tetracycline as well as vancomycin and teicoplanin, insensitive to linezolid; sensitive to tigecycline and dalfopristine.
42. Percy et al., Annu. Rev. Microbiol. 2014, 68, 81-100.
43. Brown. et al., Annu. Rev. Microbiol. 2013, 67,
44. Höltje et al., Proc. Natl. Acad. Sci., U.S.A. 1975, 72, 1690-1694.
45. Bierbaum et al., Arch. Microbiol. 1985, 141, 249-254.
46. Bierbaum et al., J. Bacteriol. 1987, 169, 5452-5458.
47. Fetterly et al., Antimicrob. Agents Chemother. 2005, 49, 148-152.
48. Xie et al., J. Am. Chem. Soc. 2012, 134, 1284-1297.
49. Xie et al., J. Am. Chem. Soc. 2011, 133, 13946-13949.
50. Okano et al., Chem. Rev. 2017, 117, 11952-11993.
51. Nakayama et al., Org. Lett. 2014, 16, 3572-3575.
52. James et al., ACS Chem. Biol. 2012, 7, 797-804.
53. Blaskovich et al., Nat. Commun. 2018, 9, 1-17.
54. Beria et al., J. Med. Chem. 2004, 47, 2611-2623.
55. Tassoni et al., J. Med. Chem. 2008, 51, 3073-3076.
56. Safir et al., J. Org. Chem., 1948, 13, 924-932.
57. Clinical and Laboratory Standards Institute, Methods for Dilution Antimicrobial Susceptibility Tests for Bacteria That Grow Aerobically, Approved Standard, 7th ed, CLSI document M07-A8, Clinical and Laboratory Standards Institute: Wayne, Pa.; 2009.
58. Savage et al., J. Antimicrob. Chemother. 2012, 67, 2665-2672.
59. Anantharaman et al., J. Med. Chem. 2010, 53, 6079-6088.
60. Boulos et al., J. Microbiol. Methods 1999, 37, 77-86.
61. Yang et al., Microbiol. Res. 2017, 195, 18-23.
62. Patch et al., J. Am. Chem. Soc. 2003, 125, 12092-12093.
63. Beria et al., J. Med. Chem. 2004, 47, 2611-2623.
64. Tassoni et al., J. Med. Chem. 2008, 51, 3073-3076.
65. Safir et al., J. Org. Chem., 1948, 13, 924-932.
66. Savage et al., J. Antimicrob. Chemother. 2012, 67, 2665-2672.
67. Anantharaman et al., J. Med. Chem. 2010, 53, 6079-6088.
68. Boulos et al., J. Microbiol. Methods 1999, 37, 77-86.
69. Yang et al., Microbiol. Res. 2017, 195, 18-23.
70. Antonoplis et al., J. Am. Chem. Soc. 2018, 140, 16140-16151.
71. Antonoplis et al., ACS Chem. Biol. 2019 14, 2065-2070.

1. A compound that corresponds in structure to that shown in Formula I or its pharmaceutically acceptable salt, 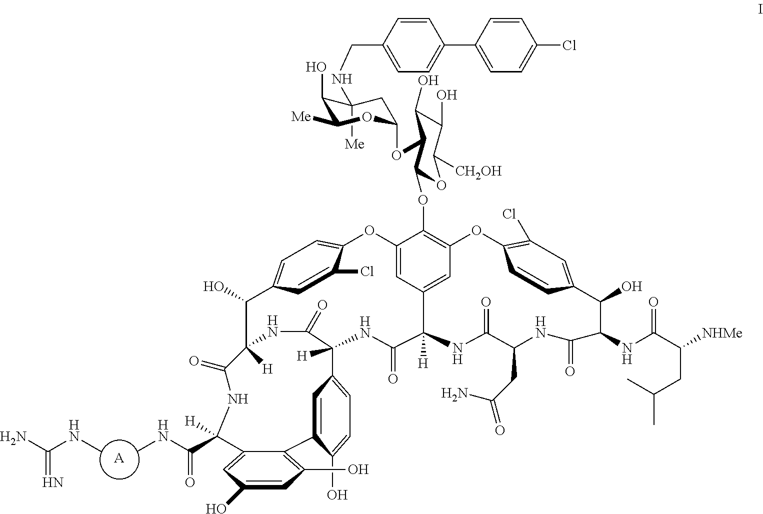
wherein
X=H,H, S, O or NH; and
Circle A is a linking moiety has a length of a saturated chain of 2 carbon atoms and less than a saturated chain of about 12 carbon atoms.

2. The compound or its pharmaceutically acceptable salt according to claim 1, wherein said Circle A linking group is comprised of two 5- or 6-membered rings, Circle B and Circle C, that are bonded together directly or indirectly in the chain of atoms that link the guanidino nitrogen atom and the amido nitrogen atom of Formula I.

3. The compound or its pharmaceutically acceptable salt according to claim 2, wherein said two 5- or 6-membered rings are directly bonded to each other.

4. The compound or its pharmaceutically acceptable salt according to claim 3, wherein said two 5- or 6-membered rings directly bonded together form a fused ring system.

5. The compound or its pharmaceutically acceptable salt according to claim 4, wherein one of the rings of said fused ring system is aromatic.

6. The compound or its pharmaceutically acceptable salt according to claim 4, wherein one of the rings of said fused ring system is carbocyclic or is heterocyclic.

7. The compound or its pharmaceutically acceptable salt according to claim 4, wherein both of the rings of said fused ring system are aromatic.

8. The compound or its pharmaceutically acceptable salt according to claim 2, wherein said Circle B and Circle C groups are indirectly bonded together by a divalent sub-linker group Z that contains one, two, three or four atoms in the chain of atoms used for determining Circle A linker length.

9. The compound or its pharmaceutically acceptable salt according to claim 8, wherein said divalent sub-linker group Z is selected from the group consisting of —O—, —S—, —NH—, —CO—NH—, —NH—CO—, —CO—O—, —O—CO—, —O—CO—O—, —C≡C—, —HC═CH—, —(CH2)n- where n=1-4, —NH—CO—NH—, —NH—CO—O—, —O—CO—NH—, —N═N—, —NH—NH—, —CS—NH—, —HN—CS—, —SO— and —SO2-.

10. The compound or its pharmaceutically acceptable salt according to claim 1, wherein said Circle A linker moiety is comprised of a hydrocarbyl chain of atoms that optionally includes one to four pendant substituents that are the same or different, are free from a carboxyl (—CO—OH) group, a carboxamido (—CO—NH2) group, a sulfhydryl (—SH) and a ring structure, and are selected from the group consisting of saturated and unsaturated natural amino acid side chain groups, 2-hydroxyethyl, 2-hydroxypropyl, C1-C3-hydrocarbyl C0-C2-carboxylate, wherein “C0” indicates that the carbonyl carbon is bonded directly to an atom of the Circle A linking moiety chain.

11. The compound or its pharmaceutically acceptable salt according to claim 10, wherein said Circle A linker moiety is selected from the group consisting of

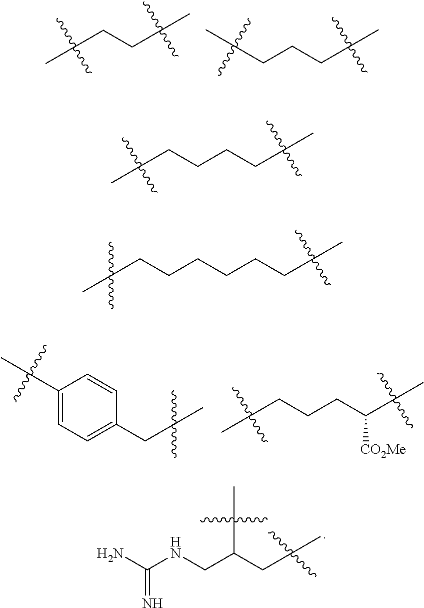

12. The compound or its pharmaceutically acceptable salt according to claim 1 that corresponds in structure to Formula Ia, Formula Ib, Formula Ic, or Formula Id, 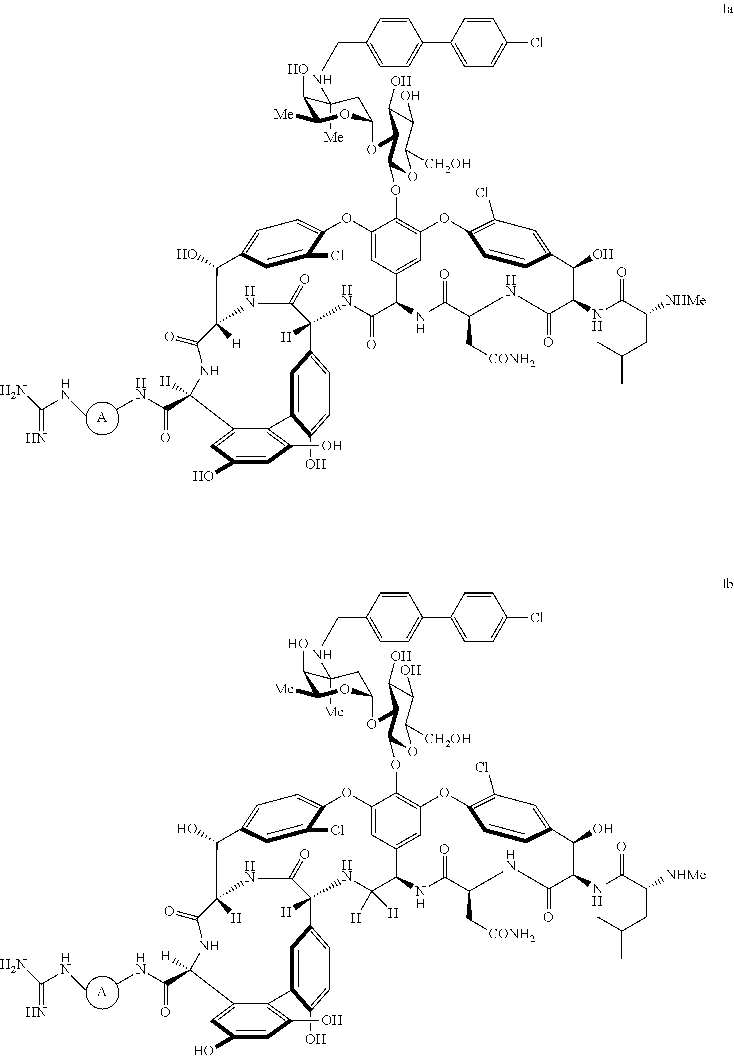 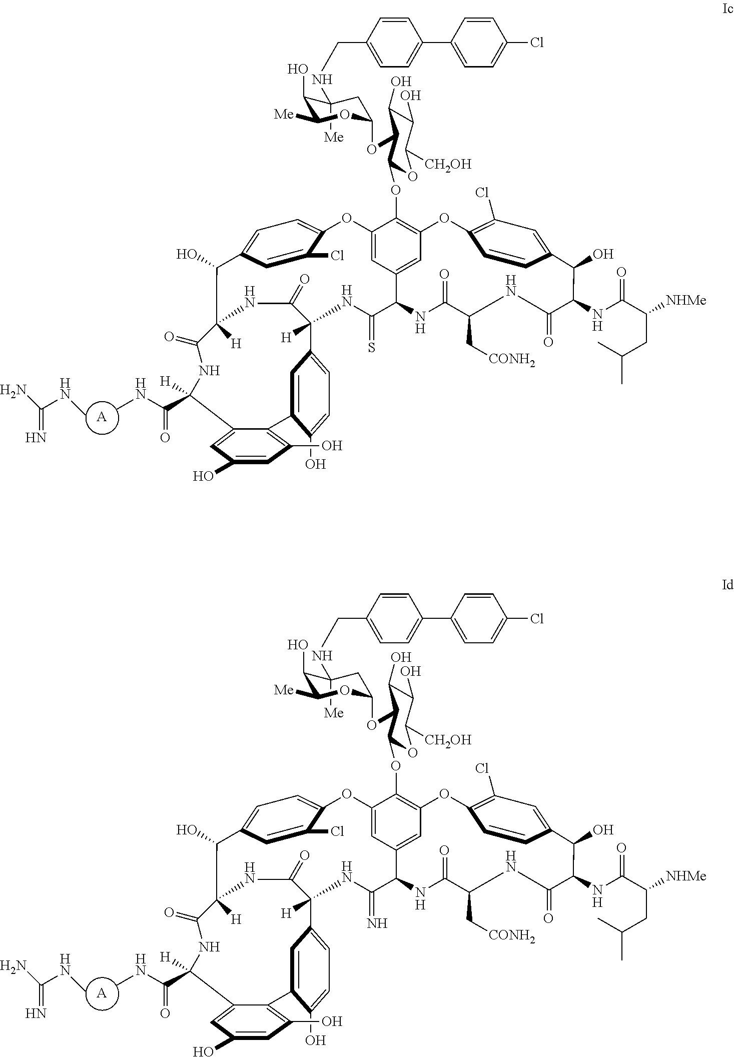
wherein the Circle A linking moiety has the length of a saturated chain of 2 carbon atoms to a saturated chain of about 10 carbon atoms.

13. The compound or its pharmaceutically acceptable salt according to claim 12, wherein said Circle A linking moiety has the length of a saturated chain of 2 carbon atoms to a saturated chain of about 8 carbon atoms.

14. A pharmaceutical composition that comprises an antimicrobial amount of a compound of claim 1 or a pharmaceutically acceptable salt thereof dissolved or dispersed in a physiologically acceptable diluent.

15. The pharmaceutical composition according to claim 14, wherein said dissolved or dispersed compound or a pharmaceutically acceptable salt thereof is a compound of Formula Ia, Ib or Id 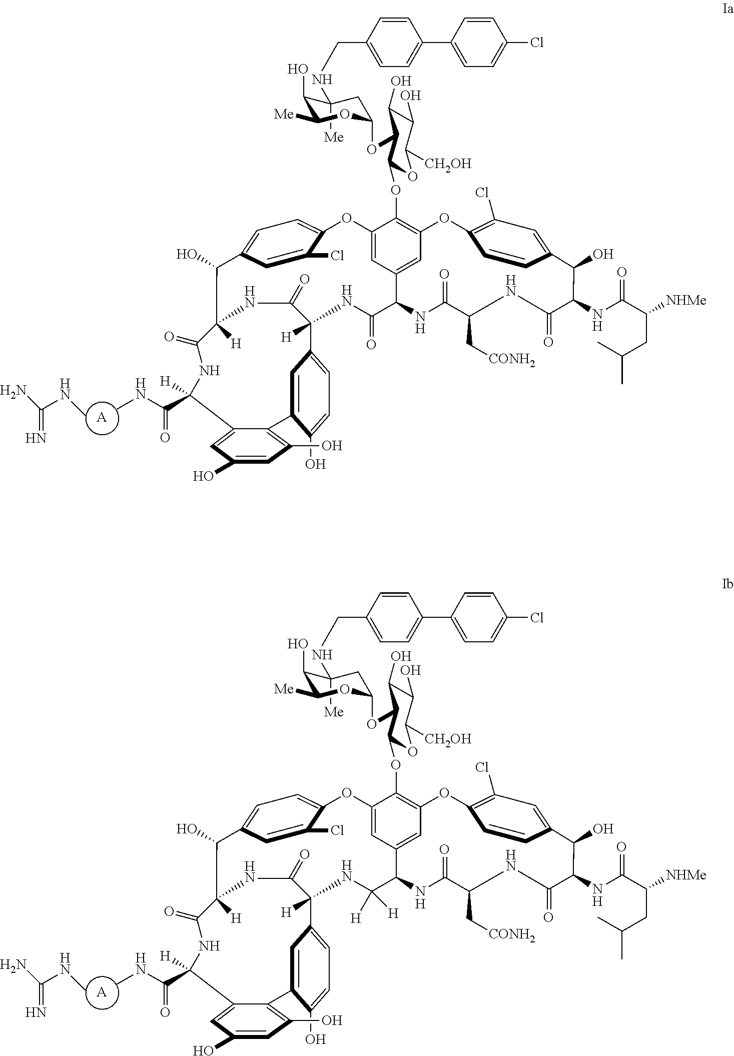 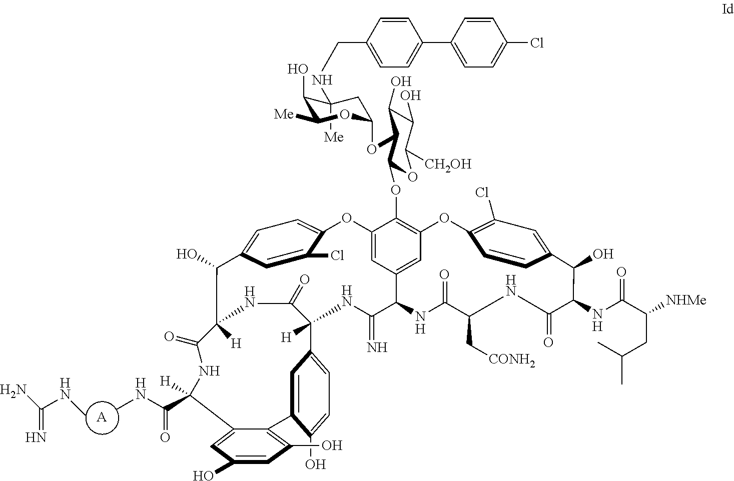
wherein the Circle A linking moiety has the length of a saturated chain of 2 carbon atoms to a saturated chain of about 10 carbon atoms.

16. The pharmaceutical composition according to claim 15, wherein said Circle A linking moiety has the length of a saturated chain of 2 carbon atoms to a saturated chain of about 8 carbon atoms.

17. The pharmaceutical composition according to claim 16, wherein said Circle A linker moiety is selected from the group consisting of

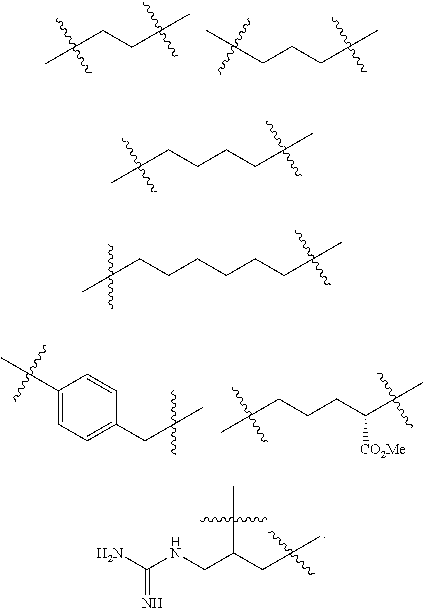

18. A method of treating a bacterially-infected mammal in need of antibacterial treatment that comprises administering an antibacterial-effective amount of a compound according to claim 1 or a pharmaceutically acceptable salt of such a compound to said infected mammal in need.

19. The method according to claim 18, wherein the bacteria that infect said bacterially-infected mammal are Gram-positive bacteria.

20. The method according to claim 19, wherein said Gram-positive bacteria are selected from the group consisting of one or more of S. aureus, methicillin-resistant S. aureus (MRSA), VanA E. faecalis, VanA E. faecium, and VanB E. faecalis.

21. The method according to claim 18, wherein said administration is repeated a plurality of times.

22. The method according to claim 18, wherein the bacteria that infect said bacterially-infected mammal are Gram-negative bacteria.

23. The method according to claim 20, wherein said administration is repeated a plurality of times.
